(19) United States
(12) Patent Application Publication
(10) Pub. No.: US 2024/0360081 A1
(43) Pub. Date: Oct. 31, 2024

(54) SULFONAMIDE OREXIN RECEPTOR AGONISTS AND USES THEREOF

(71) Applicant: Jazz Pharmaceuticals Ireland Limited, Dublin (IE)

(72) Inventors: Davide MARINELLI, Verona (IT); Prafulkumar Chovatia, Abingdon (GB); Claudia BEATO, Castel d'Azzano (IT); Gilles OUVRY, Abingdon (GB)

(21) Appl. No.: 18/571,860

(22) PCT Filed: Jun. 24, 2022
(86) PCT No.: PCT/EP2022/067376

§ 371 Date: Dec. 19, 2023

Related U.S. Application Data
(60) Provisional application No. 63/215,054, filed on Jun. 25, 2021.

(51) Int. Cl. C07D 213/64; A61K 31/415; A61K 31/4192; A61K 31/437; A61K 31/44; A61K 31/4412; A61K 31/443; A61K 31/4439; A61K 31/444; A61K 31/4704; A61K 31/506; A61K 31/513; C07D 213/84; C07D 215/227; C07D 231/12; C07D 239/36; C07D 249/18; C07D 401/04; C07D 401/12; C07D 401/14; C07D 405/12; C07D 471/04

(52) U.S. Cl. CPC C07D 213/64; A61K 31/415; A61K 31/4192; A61K 31/437; A61K 31/44; A61K 31/4412; A61K 31/443; A61K 31/4439; A61K 31/444; A61K 31/4704; A61K 31/506; A61K 31/513; C07D 213/84; C07D 215/227; C07D 231/12; C07D 239/36; C07D 249/18; C07D 401/04; C07D 401/12; C07D 401/14; C07D 405/12; C07D 471/04

(57) ABSTRACT

Provided herein are compounds of Formula (I), or pharmaceutically acceptable salt thereof, wherein R₁, R₂, R₃, R₄, A, L₁, L₂, Y and Z are defined herein. Also provided herein are pharmaceutical compositions comprising a compound of Formula (I) or pharmaceutically acceptable salt thereof, and methods of using a compound of Formula (I) or pharmaceutically acceptable salt thereof, e.g., in the treatment of a disease or disorder that is treatable by administration of an Orexin agonist.

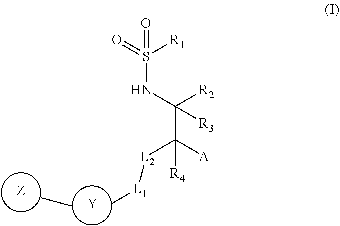

### CROSS-REFERENCE TO RELATED APPLICATIONS

This application claims priority to U.S. Provisional Application No. 63/215,054, filed on Jun. 25, 2021, the contents of which is hereby incorporated by reference in its entirety for all purposes.

### BACKGROUND

Orexin is a neuropeptide specifically produced in particular neurons located sparsely in the lateral hypothalamus and its surrounding area. Orexin consists of two subtypes, orexin A and orexin B. Both orexin A (OX-A) and orexin B (OX—B) are endogenous ligands of the orexin receptors, which are mainly present in the brain. Two orexin receptors have been cloned and characterized in mammals. They belong to the super family of G-protein coupled receptors: the orexin-1 receptor (OX or OX1R) is partially selective for OX-A and the orexin-2 receptor (OX2 or OX2R) is capable of binding OX-A as well as OX—B with similar affinity. The physiological actions in which orexins are presumed to participate are thought to be expressed via one or both of OX1 receptor and OX2 receptor as the two subtypes of orexin receptors.

Orexins regulate states of sleep and wakefulness making the orexin system a target for potential therapeutic approaches to treat sleep disorders. Orexins are found to stimulate food consumption in rats suggesting a physiological role for these peptides as mediators in the central feedback mechanism that regulates feeding behavior. Orexins have also been indicated as playing a role in arousal, emotion, energy homeostasis, reward, learning and memory.

There is a need for compounds that modulate orexin receptors, as well as compositions and methods for treating a disease or disorder that is treatable by administration of an orexin agonist.

### SUMMARY

The present disclosure is directed to compounds that are agonists of the orexin-2 receptor as well as pharmaceutical compositions thereof and uses thereof in treating a disease or disorder that is treatable by administration of an orexin agonist.

In one aspect, provided herein are compounds of Formula (I):

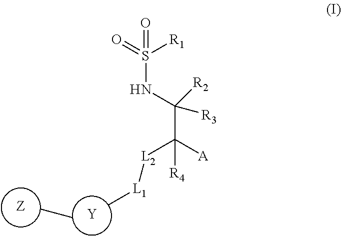

or a pharmaceutically acceptable salt or N-oxide thereof,
wherein
A is a heteroaryl;
L₁ is —O—, —CR₅R₆—or a bond;
L₂ is —CR₅R₆;
R₁ is alkyl, cycloalkyl, heterocyclyl, aryl, heteroaryl, or —NR₇R₈, or R₁ and R₂ together with the atom to which they are attached form a heterocycle;
R₂, R₃, R₄ are independently hydrogen, alkyl, cycloalkyl, heterocyclyl, or halogen, or R₂ and R₃ together with the atom to which they are attached to form a carbocycle or heterocycle, or R₂ and R₄ together with the atom to which they are attached to form a carbocycle or heterocycle;
R₅ and R₆ are each independently hydrogen, alkyl, cycloalkyl, heterocyclyl, alkoxy, —O-cycloalkyl, —O-heterocyclyl, or halogen, or R₅ and R₆ together with the atom to which they are attached to form a carbocycle or heterocycle;
R₇ and R₈ are independently hydrogen, alkyl, cycloalkyl, or heterocyclyl, or R₇ and R₈ together with the atom to which they are attached form a heterocycle;
Y is cycloalkyl, heterocyclyl, heteroaryl or aryl; and
Z is absent, heteroaryl or aryl, wherein if L₁ is a bond or CR₅R₆ then Z is heteroaryl or aryl; and with the proviso that the compound is not:

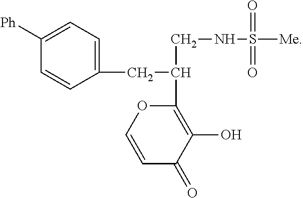 [SMILES: CS(=O)(=O)NCC(Cc1ccc(-c2ccccc2)cc1)c1occc(=O)c1O]

In one aspect, provided herein are compounds of Formula (I):

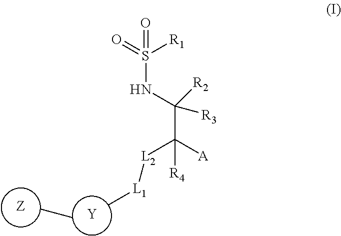

or a pharmaceutically acceptable salt thereof,
wherein
A is a heteroaryl;
L₁ is —O—, —CR₅R₆—or a bond;
L₂ is —CR₅R₆;
R₁ is alkyl, cycloalkyl, heterocyclyl, aryl, heteroaryl, or —NR₇R₈, or R₁ and R₂ together with the atom to which they are attached form a heterocycle;
R₂, R₃, R₄ are independently hydrogen, alkyl, cycloalkyl, heterocyclyl, or halogen, or R₂ and R₃ together with the atom to which they are attached to form a carbocycle or heterocycle, or R₂ and R₄ together with the atom to which they are attached to form a carbocycle or heterocycle;
R₅ and R₆ are each independently hydrogen, alkyl, cycloalkyl, heterocyclyl, alkoxy, —O-cycloalkyl, —O-heterocyclyl, or halogen, or R₅ and R₆ together with the atom to which they are attached to form a carbocycle or heterocycle;
R₇ and R₈ are independently hydrogen, alkyl, cycloalkyl, or heterocyclyl, or R₇ and R₈ together with the atom to which they are attached form a heterocycle;
Y is cycloalkyl, heterocyclyl, heteroaryl or aryl; and
Z is absent, heteroaryl or aryl, wherein if L₁ is a bond or CR₅R₆ then Z is heteroaryl or aryl; and with the proviso that the compound is not:

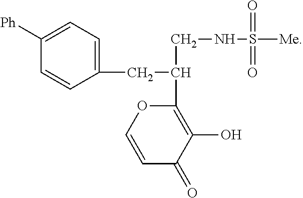 [SMILES: CS(=O)(=O)NCC(Cc1ccc(-c2ccccc2)cc1)c1occc(=O)c1O]

In some embodiments, the present disclosure provides a compound of Formula (I-A)

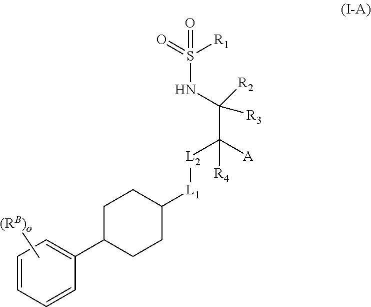

or a pharmaceutically acceptable salt thereof, wherein o, A, L₁, L₂, R₁, R₂, R₃, R₄, and R^{B} are defined herein.

In some embodiments, the present disclosure provides a compound of Formula (I-A-I)

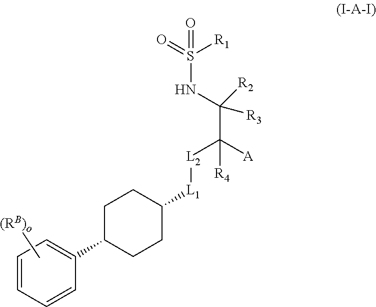

or a pharmaceutically acceptable salt thereof, wherein o, A, L₁, L₂, R₁, R₂, R₃, R₄, and R^{B} are defined herein.

In some embodiments, the present disclosure provides a compound of Formula (I-B):

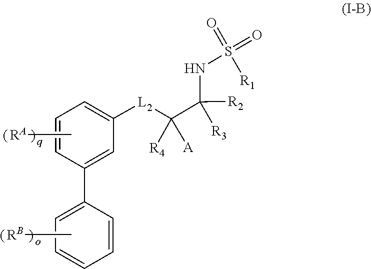

or a pharmaceutically acceptable salt thereof, wherein o, q, A, L₂, R₁, R₂, R₃, R₄, R^{A}, R^{B} are defined herein.

In some embodiments, the present disclosure provides a pharmaceutical composition comprising one or more compounds of the present disclosure or a pharmaceutically acceptable salt thereof and a pharmaceutically acceptable carrier.

In some embodiments, the present disclosure provides a pharmaceutical composition comprising one or more compounds of the present disclosure or a pharmaceutically acceptable salt or N-oxide thereof and a pharmaceutically acceptable carrier.

In some embodiments, the present disclosure provides a method of treating a disease or disorder that is treatable by administration of an orexin agonist comprising administering a therapeutically effective amount of one or more compounds of the present disclosure or a pharmaceutically acceptable salt thereof.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The term “treating” as used herein with regard to a patient, refers to improving at least one symptom of the patient's disorder. Treating can be improving, or at least partially ameliorating a disorder or an associated symptom of a disorder.

The terms “effective amount” and “therapeutically effective amount” are used interchangeably in this disclosure and refer to an amount of a compound, or a salt thereof, (or pharmaceutical composition containing the compound or salt) that, when administered to a patient, is capable of performing the intended result. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

The term “carrier” or “vehicle” as used interchangeably herein encompasses carriers, excipients, adjuvants, and diluents or a combination of any of the foregoing, meaning a material, composition or vehicle, such as a liquid or solid filler, diluent, excipient, solvent or encapsulating material involved in carrying or transporting a pharmaceutical agent from one organ, or portion of the body, to another organ or portion of the body. In addition to the adjuvants, excipients and diluents known to one skilled in the art, the carrier includes nanoparticles of organic and inorganic nature.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Aralkyl” or “arylalkyl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, including compounds with aromatic resonance structures (e.g., 2-pyridone), and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Heterocyclylalkyl” refers to a radical of the formula —R_{b}—Rₑ where R_{b} is an alkylene, alkenylene, or alkynylene group as defined above and Rₑ is a heterocyclyl radical as defined above. Unless stated otherwise specifically in the specification, a heterocyclylalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, =NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In some embodiments, “substituted” further means any alkyl, cycloalkyl or heterocyclylalkyl in which one or more hydrogen atoms is replaced by an isotope e.g., deuterium. In addition, each of the foregoing substituents can also be optionally substituted with one or more of the above substituents.

As used herein, the symbol “

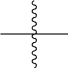

” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “

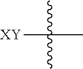

” indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or “

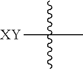

” infers that when R³ is “XY”, the point of attachment bond is the same bond as the bond by which R₃ is depicted as being bonded to CH₃.

### Compounds

The present disclosure provides compounds that are agonists of the orexin type 2 receptor as well as pharmaceutical compositions thereof and uses thereof in treating various diseases and disorders.

In embodiments, the present disclosure provides a compound of Formula (I):

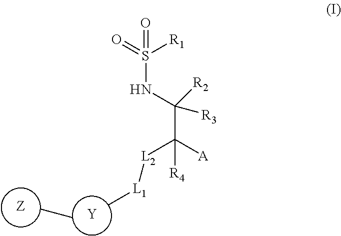

or a pharmaceutically acceptable salt or N-oxide thereof,
wherein
A is a heteroaryl;
L₁ is —O—, —CR₅R₆— or a bond;
L₂ is —CR₅R₆;
R₁ is alkyl, cycloalkyl, heterocyclyl, aryl, heteroaryl, or —NR₇R₈, or R₁ and R₂ together with the atom to which they are attached form a heterocycle;
R₂, R₃, R₄ are independently hydrogen, alkyl, cycloalkyl, heterocyclyl, or halogen, or R₂ and R₃ together with the atom to which they are attached to form a carbocycle or heterocycle, or R₂ and R₄ together with the atom to which they are attached to form a carbocycle or heterocycle;
R₅ and R₆ are each independently hydrogen, alkyl, cycloalkyl, heterocyclyl, alkoxy, —O-cycloalkyl, —O-heterocyclyl, or halogen, or R₅ and R₆ together with the atom to which they are attached to form a carbocycle or heterocycle;
R₇ and R₈ are independently hydrogen, alkyl, cycloalkyl, or heterocyclyl, or R₇ and R₈ together with the atom to which they are attached form a heterocycle;
Y is cycloalkyl, heterocyclyl, heteroaryl or aryl; and
Z is absent, heteroaryl or aryl, wherein if L₁ is a bond or CR₅R₆ then Z is heteroaryl or aryl; and with the proviso that the compound is not:

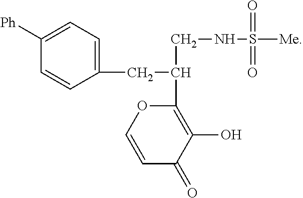 [SMILES: CS(=O)(=O)NCC(Cc1ccc(-c2ccccc2)cc1)c1occc(=O)c1O]

In some embodiments, the present disclosure provides a compound of Formula (I):

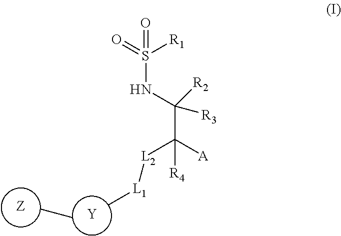

or a pharmaceutically acceptable salt thereof,
wherein
A is a heteroaryl;
L₁ is —O—, —CR₅R₆—or a bond;
L₂ is —CR₅R₆;
R₁ is alkyl, cycloalkyl, heterocyclyl, aryl, heteroaryl, or —NR₇R₈, or R₁ and R₂ together with the atom to which they are attached form a heterocycle;
R₂, R₃, R₄ are independently hydrogen, alkyl, cycloalkyl, heterocyclyl, or halogen, or R₂ and R₃ together with the atom to which they are attached to form a carbocycle or heterocycle, or R₂ and R₄ together with the atom to which they are attached to form a carbocycle or heterocycle;
R₅ and R₆ are each independently hydrogen, alkyl, cycloalkyl, heterocyclyl, alkoxy, —O-cycloalkyl, —O-heterocyclyl, or halogen, or R₅ and R₆ together with the atom to which they are attached to form a carbocycle or heterocycle;
R₇ and R₈ are independently hydrogen, alkyl, cycloalkyl, or heterocyclyl, or R₇ and R₈ together with the atom to which they are attached form a heterocycle;
Y is cycloalkyl, heterocyclyl, heteroaryl or aryl; and
Z is absent, heteroaryl or aryl, wherein if L₁ is a bond or CR₅R₆ then Z is heteroaryl or aryl; and with the proviso that the compound is not:

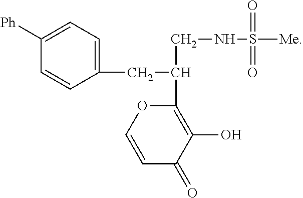 [SMILES: CS(=O)(=O)NCC(Cc1ccc(-c2ccccc2)cc1)c1occc(=O)c1O]

In some embodiments, the present disclosure provides a compound of Formula (I-A)

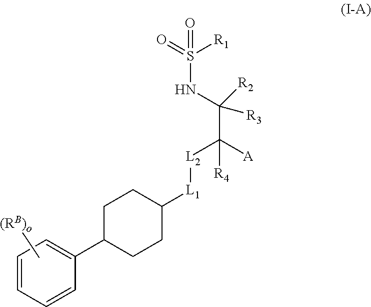

or a pharmaceutically acceptable salt thereof, wherein o, A, L₁, L₂, R₁, R₂, R₃, R₄, and R^{B} are defined herein.

In some embodiments, the present disclosure provides a compound of Formula (I-A)

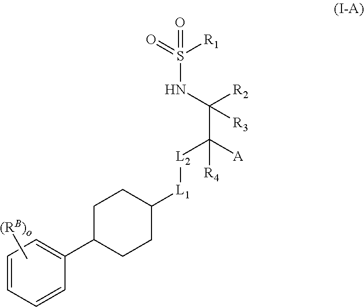

or a pharmaceutically acceptable salt thereof, wherein:
A is a heteroaryl;
L₁ is —O—, —CR₅R₆—or a bond;
L₂ is —CR₅R₆;
R₁ is alkyl, cycloalkyl, heterocyclyl, aryl, heteroaryl, or —NR₇R₈, or R₁ and R₂ together with the atom to which they are attached form a heterocycle;
R₂, R₃, R₄ are independently hydrogen, alkyl, cycloalkyl, heterocyclyl, or halogen, or R₂ and R₃ together with the atom to which they are attached to form a carbocycle or heterocycle, or R₂ and R₄ together with the atom to which they are attached to form a carbocycle or heterocycle;
R₅ and R₆ are each independently hydrogen, alkyl, cycloalkyl, heterocyclyl, alkoxy, —O— cycloalkyl, —O-heterocyclyl, or halogen, or R₅ and R₆ together with the atom to which they are attached to form a carbocycle or heterocycle;
R₇ and R₈ are independently hydrogen, alkyl, cycloalkyl, or heterocyclyl, or R₇ and R₈ together with the atom to which they are attached form a heterocycle;
R^{B} is independently, for each occurrence, selected from the group consisting of hydroxy, halo, —NO₂, —CN, —NR₇R₈, —CO₂R₁₀, —OC(O)R₁₀, —COR₁₀, —C(O)NR₇R₈, —NR₇C(O)R₁₀, —OC(O)NR₇R₈, —NR₇C(O)OR₁₀, —S(O)_{w}R₁₀ (wherein w is 0, 1, or 2), —OSO₂R₁₀, —SO₃R₁₀, —S(O)₂NR₇R₈, —NR₇S(O)₂R₁₀, —NR₇C(O)NR₇R₈, —C₁₋₆ alkyl-NR₇R₈, —C₁₋₆ alkyl-O—C₁₋₆ alkyl, —C₁₋₆ alkyl, C₁₋₆ alkoxy, C₂₋₆ alkenyl, and C₂₋₆ alkynyl;
R₁₀ is independently selected, for each occurrence, from the group consisting of hydrogen, C₁₋₆alkyl, C₂₋₆alkenyl, C₂₋₆alkynyl, aryl, cycloalkyl, heterocyclyl, and heteroaryl; and
o is 0, 1, 2, 3, 4, or 5.

In some embodiments, the present disclosure provides a compound of Formula (I-A-I)

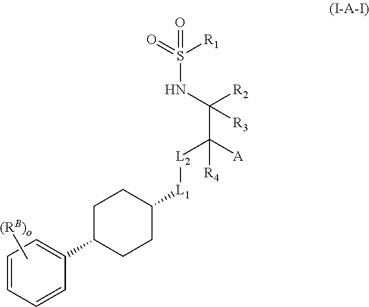

or a pharmaceutically acceptable salt thereof, wherein o, A, L₁, L₂, R₁, R₂, R₃, R₄, and R^{B} are defined herein.

In some embodiments, the present disclosure provides a compound of Formula (I-A-I)

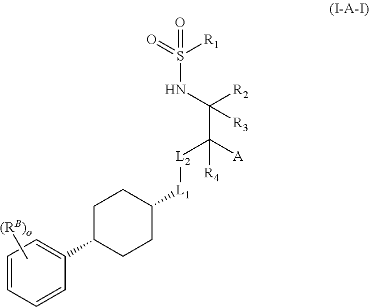

or a pharmaceutically acceptable salt thereof, wherein:
A is a heteroaryl;
L₁ is —O—, —CR₅R₆—or a bond;
L₂ is —CR₅R₆;
R₁ is alkyl, cycloalkyl, heterocyclyl, aryl, heteroaryl, or —NR₇R₈, or R₁ and R₂ together with the atom to which they are attached form a heterocycle;
R₂, R₃, R₄ are independently hydrogen, alkyl, cycloalkyl, heterocyclyl, or halogen, or R₂ and R₃ together with the atom to which they are attached to form a carbocycle or heterocycle, or R₂ and R₄ together with the atom to which they are attached to form a carbocycle or heterocycle;
R₅ and R₆ are each independently hydrogen, alkyl, cycloalkyl, heterocyclyl, alkoxy, —O— cycloalkyl, —O-heterocyclyl, or halogen, or R₅ and R₆ together with the atom to which they are attached to form a carbocycle or heterocycle;
R₇ and R₈ are independently hydrogen, alkyl, cycloalkyl, or heterocyclyl, or R₇ and R₈ together with the atom to which they are attached form a heterocycle;
R^{B} is independently, for each occurrence, selected from the group consisting of hydroxy, halo, —NO₂, —CN, —NR₇R₈, —CO₂R₁₀, —OC(O)R₁₀, —COR₁₀, —C(O)NR₇R₈, —NR₇C(O)R₁₀, —OC(O)NR₇R₈, —NR₇C(O)OR₁₀, —S(O)_{w}R₁₀ (wherein w is 0, 1, or 2), —OSO₂R₁₀, —SO₃R₁₀, —S(O)₂NR₇R₈, —NR₇S(O)₂R₁₀, —NR₇C(O)NR₇R₈, —C₁₋₆ alkyl-NR₇R₈, —C₁₋₆ alkyl-O—C₁₋₆ alkyl, —C₁₋₆ alkyl, C₁₋₆ alkoxy, C₂₋₆ alkenyl, and C₂₋₆ alkynyl;
R₁₀ is independently selected, for each occurrence, from the group consisting of hydrogen, C₁₋₆alkyl, C₂₋₆alkenyl, C₂₋₆alkynyl, aryl, cycloalkyl, heterocyclyl, and heteroaryl; and
o is 0, 1, 2, 3, 4, or 5.

In some embodiments, the present disclosure provides a compound of Formula (I-B):

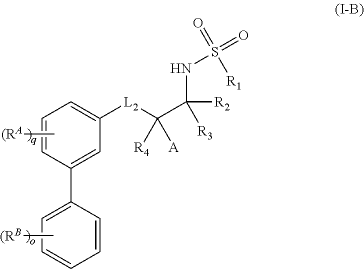

or a pharmaceutically acceptable salt thereof, wherein o, q, A, L₂, R₁, R₂, R₃, R₄, R^{A}, and
R^{B} are defined herein.

In some embodiments, the present disclosure provides a compound of Formula (I-B):

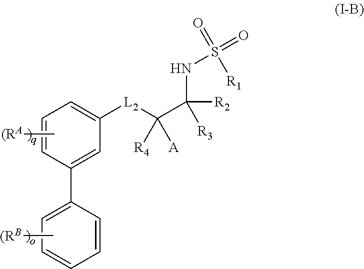

or a pharmaceutically acceptable salt thereof, wherein:
A is a heteroaryl;
L₂ is —CR₅R₆;
R₁ is alkyl, cycloalkyl, heterocyclyl, aryl, heteroaryl, or —NR₇R₈, or R₁ and R₂ together with the atom to which they are attached form a heterocycle;
R₂, R₃, R₄ are independently hydrogen, alkyl, cycloalkyl, heterocyclyl, or halogen, or R₂ and R₃ together with the atom to which they are attached to form a carbocycle or heterocycle, or R₂ and R₄ together with the atom to which they are attached to form a carbocycle or heterocycle;
R₅ and R₆ are each independently hydrogen, alkyl, cycloalkyl, heterocyclyl, alkoxy, —O— cycloalkyl, —O-heterocyclyl, or halogen, or R₅ and R₆ together with the atom to which they are attached to form a carbocycle or heterocycle;
R₇ and R₈ are independently hydrogen, alkyl, cycloalkyl, or heterocyclyl, or R₇ and R₈ together with the atom to which they are attached form a heterocycle;
R^{A} and R^{B} are independently, for each occurrence, selected from the group consisting of hydroxy, halo, —NO₂, —CN, —NR₇R₈, —CO₂R₁₀, —OC(O)R₁₀, —COR₁₀, —C(O)NR₇R₈, —NR₇C(O)R₁₀, —OC(O)NR₇R₈, —NR₇C(O)OR₁₀, —S(O)_{w}R₁₀ (wherein w is 0, 1, or 2), —OSO₂R₁₀, —SO₃R₁₀, —S(O)₂NR₇R₈, —NR₇S(O)₂R₁₀, —NR₇C(O)NR₇R₈, —C₁₋₆ alkyl-NR₇R₈, —C₁₋₆ alkyl-O—C₁₋₆ alkyl, —C₁₋₆ alkyl, C₁₋₆ alkoxy, C₂₋₆ alkenyl, and C₂₋₆ alkynyl;
R₁₀ is independently selected, for each occurrence, from the group consisting of hydrogen, C₁₋₆alkyl, C₂₋₆alkenyl, C₂₋₆alkynyl, aryl, cycloalkyl, heterocyclyl, and heteroaryl;
q is 0, 1, 2, 3, or 4; and
o is 0, 1, 2, 3, 4, or 5.

In some embodiments of the compounds of Formula (I), (I-A), (I-A-I), and (I-B), A is a heteroaryl.

In some embodiments of the compounds of Formula (I), (I-A), (I-A-I), and (I-B), A is a substituted or unsubstituted heteroaryl containing at least one nitrogen atom. In some embodiments A contains one nitrogen atom. In some embodiments, A contains two nitrogen atoms.

In some embodiments of the compounds of Formula (I), (I-A), (I-A-I), and (I-B), A is a substituted or unsubstituted monocyclic or bicyclic nitrogen-containing heteroaryl.

In embodiments of the compounds of Formula (I), (I-A), (I-A-I), and (I-B), A is selected from the group consisting of:

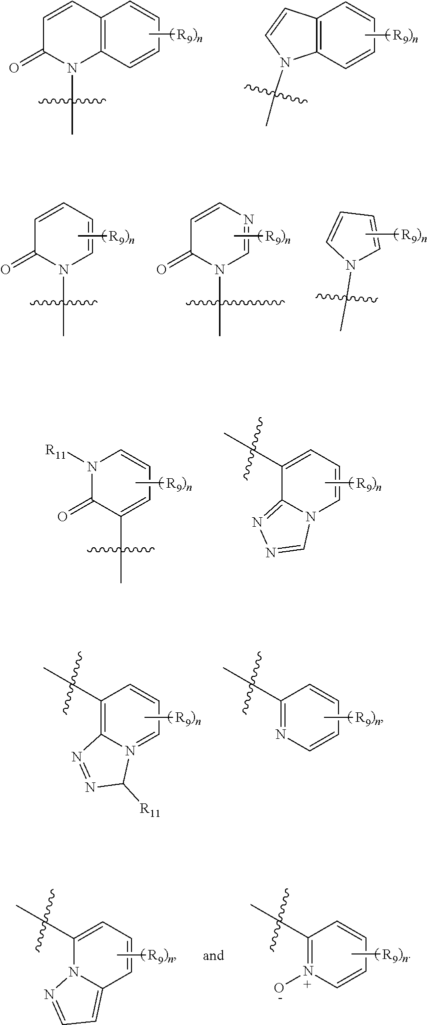

In some embodiments of the compounds of Formula (I), (I-A), (I-A-I), and (I-B), A is selected from the group consisting of:

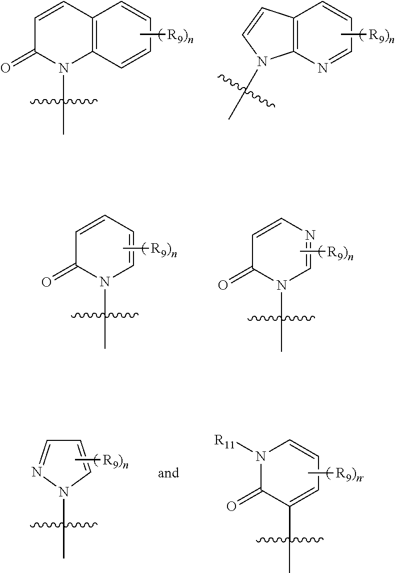

In some embodiments of the compounds of Formula (I), (I-A), (I-A-I), and (I-B), A is

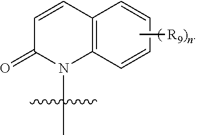

In some embodiments, A is

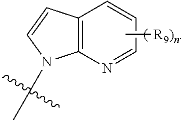

In some embodiments of the compounds of Formula (I), (I-A), (I-A-I), and (I-B), A is

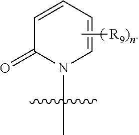

In some embodiments of the compounds of Formula (I), (I-A), (I-A-I), and (I-B), A is

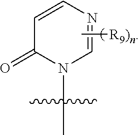

In some embodiments of the compounds of Formula (I), (I-A), (I-A-I), and (I-B), A is

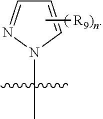

In some embodiments of the compounds of Formula (I), (I-A), (I-A-I), and (I-B), A is

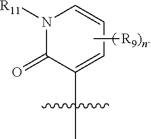

In embodiments of the compounds of Formula (I), (I-A), (I-A-I), and (I-B), A is

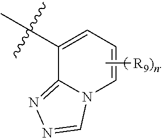

In embodiments of the compounds of Formula (I), (I-A), (I-A-I), and (I-B), A is

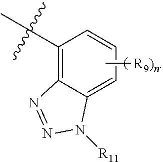

In embodiments of the compounds of Formula (I), (I-A), (I-A-I), and (I-B), A is

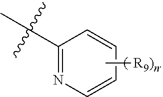

In embodiments of the compounds of Formula (I), (I-A), (I-A-I), and (I-B), A is

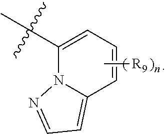

In embodiments of the compounds of Formula (I), (I-A), (I-A-I), and (I-B), A is

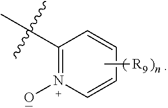

In embodiments of the compounds of Formula (I), (I-A), (I-A-I), and (I-B), R₁₁ is alkyl optionally substituted with cyano, haloalkyl, cycloalkyl or heterocyclyl.

In embodiments of the compounds of Formula (I), (I-A), (I-A-I), and (I-B), R₁₁ is alkyl optionally substituted with cyano. In embodiments, Ru is haloalkyl. In embodiments, Ru is C₃₋₆ cycloalkyl. In embodiments, R₁₁ is heterocyclyl.

In some embodiments of the compounds of Formula (I), (I-A), (I-A-I), and (I-B), R¹¹ is alkyl, cycloalkyl or heterocyclyl.

In embodiments, of the compounds of Formula (I), (I-A), (I-A-I), and (I-B), Ru is alkyl or haloalkyl.

In some embodiments of the compounds of Formula (I), (I-A), (I-A-I), and (I-B), R¹¹ is substituted alkyl. In some embodiments, R¹¹ is unsubstituted alkyl. In some embodiments, R¹¹ is alkyl optionally substituted with one or more deuterium atoms.

In some embodiments of the compounds of Formula (I), (I-A), (I-A-I), and (I-B), R¹¹ is methyl, —CH₂CN, cyclopropyl, —CH₂CF₂H, or —CF₂H.

In some embodiments of the compounds of Formula (I), (I-A), (I-A-I), and (I-B), n is an integer from 0-6 (i.e. 0, 1, 2, 3, 4, 5, or 6). In some embodiments, n is an integer from 0-4, or 1-4. In some embodiments, n is an integer from 0-3, or 1-3. In embodiments, n is 0, 1, or 2. In some embodiments, n is 1 or 2. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments of the compounds of Formula (I), (I-A), (I-A-I), and (I-B), A is optionally substituted with one more R₉. In some embodiments, A is substituted with R₉. In some embodiments, A is unsubstituted.

In some embodiments of the compounds of Formula (I), (I-A), (I-A-I), and (I-B), R₉ is independently for each occurrence hydrogen, alkyl, halogen, cyano, alkoxy, cycloalkyl, heterocyclyl, or heteroaryl. In some embodiments R₉ is alkyl optionally substituted with one or more deuterium atoms.

In some embodiments of the compounds of Formula (I), (I-A), (I-A-I), and (I-B), R₉ is independently for each occurrence alkyl, halogen, cyano, alkoxy, or heteroaryl.

In some embodiments of the compounds of Formula (I), (I-A), (I-A-I), and (I-B), R₉ is independently for each occurrence —CH₃, —OCH₃, —CH₂CH₃, —CF₃, —OCHF₂, —Br, —Cl, —F, cyano, pyridinyl, or pyrazolyl.

In some embodiments of the compounds of Formula (I), (I-A), (I-A-I), and (I-B), R₉ is independently for each occurrence hydrogen, alkyl, halogen, cyano, or alkoxy.

In some embodiments of the compounds of Formula (I), (I-A), (I-A-I), and (I-B), R₉ is independently for each occurrence alkyl, halogen, cyano, or alkoxy. In embodiments, R₉ is alkyl or halogen.

In some embodiments of the compounds of Formula (I), (I-A), (I-A-I), and (I-B), R₉ is independently for each occurrence C₁₋₃alkyl, C₁₋₃alkoxy, —Br, —Cl, —F, and cyano. In some embodiments, C₁₋₃alkyl and C₁₋₃alkoxy are optionally substituted with one or more —F.

In some embodiments of the compounds of Formula (I), (I-A), (I-A-I), and (I-B), R₉ is independently for each occurrence —CH₃, —OCH₃, —CH₂CH₃, —CF₃, —OCHF₂, —Br, —Cl, —F, or cyano.

In some embodiments of the compounds of Formula (I), (I-A), (I-A-I), and (I-B), n is 2 and R₉ is independently C₁₋₃alkyl or halogen. In some embodiments, R₉ is unsubstituted C₁₋₃alkyl. In some embodiments, R₉ is methyl. In some embodiments, R₉ is —Cl.

In embodiments of the compounds of Formula (I), (I-A), (I-A-I), and (I-B), A is selected from the group consisting of:

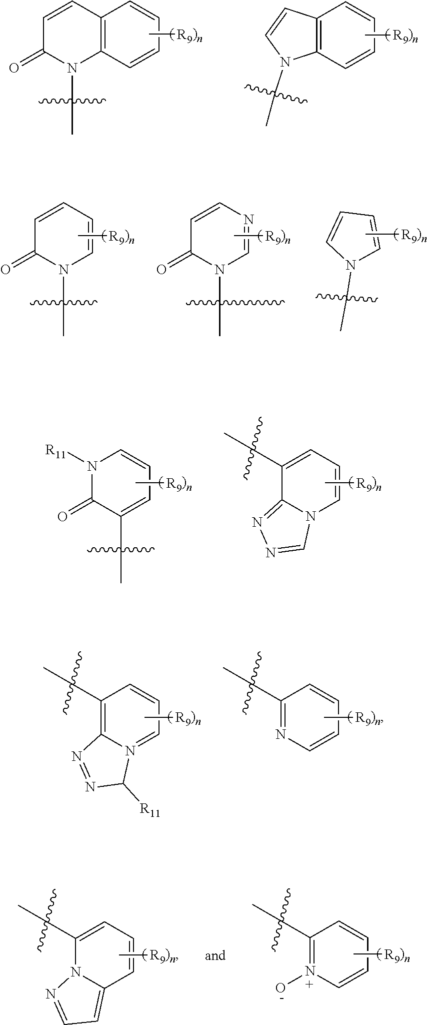

wherein n is an integer from 0-6;
R₉ is hydrogen, alkyl, haloalkyl, haloalkoxy, halogen, cyano, alkoxy, cycloalkyl, heterocyclyl, or heteroaryl; and
R₁₁ is alkyl optionally substituted with cyano, haloalkyl, cycloalkyl or heterocyclyl.

In some embodiments of the compounds of Formula (I), (I-A), (I-A-I), and (I-B), A is selected from the group consisting of:

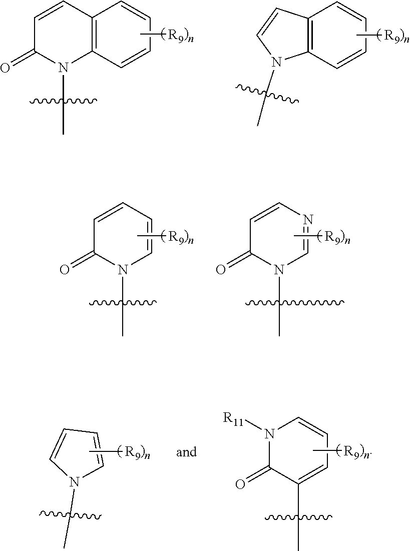

wherein n is an integer from 0-6;
R₉ is hydrogen, alkyl, halogen, cyano, alkoxy, cycloalkyl, heterocyclyl, or heteroaryl; and
R₁₁ is alkyl, cycloalkyl or heterocyclyl.

In some embodiments of the compounds of Formula (I), (I-A), (I-A-I), and (I-B), n is 1 and R₉ is —CH₃, —OCH₃, —CH₂CH₃, —CF₃, —OCHF₂, —Br, —Cl, —F, or cyano. In some embodiments, n is 2 and R₉ is independently heteroaryl, —CH₃ or halo.

In some embodiments of the compounds of Formula (I), (I-A), and (I-A-I), L₁ is —O—, —CR₅R₆—or a bond. In some embodiments, L₁ is —O—. In some embodiments, L₁ is a bond. In some embodiments, L₁ is —CR₅R₆—.

In embodiments of the compounds of Formula (I), (I-A), (I-A-I), and (I-B), L₂ is —CR₅R₆.

In some embodiments of the compounds of Formula (I), (I-A), (I-A-I), and (I-B), R₁ is alkyl, cycloalkyl, heterocyclyl, aryl, heteroaryl, or —NR₇R₈, or R₁ and R₂ together with the atom to which they are attached form a heterocycle.

In some embodiments of the compounds of Formula (I), (I-A), (I-A-I), and (I-B), R₁ is alkyl, cycloalkyl, heterocyclyl, aryl, heteroaryl, or —NR₇R₈.

In embodiments of the compounds of Formula (I), (I-A), (I-A-I), and (I-B), R₁ is an alkyl, haloalkyl, cycloalkyl, alkylene-alkoxy, or —NR₇R₈.

In embodiments of the compounds of Formula (I), (I-A), (I-A-I), and (I-B), R₁ is methyl, —N(CH₃)₂, cyclopropyl, —CH₂CF₂H, —CH₂CF₃, —CH₂CH₂F, or —CH₂CH₂OMe.

In embodiments of the compounds of Formula (I), (I-A), (I-A-I), and (I-B), R₁ is an alkyl, haloalkyl, or —NR₇R₈.

In some embodiments of the compounds of Formula (I), (I-A), (I-A-I), and (I-B), R₁ is alkyl or —NR₇R₈,

In some embodiments of the compounds of Formula (I), (I-A), (I-A-I), and (I-B), R₁ is alkyl.

In some embodiments of the compounds of Formula (I), (I-A), (I-A-I), and (I-B), R₁ is methyl.

In some embodiments of the compounds of Formula (I), (I-A), (I-A-I), and (I-B), R₁ is —NR₇R₈.

In some embodiments of the compounds of Formula (I), (I-A), (I-A-I), and (I-B), R₂, R₃, R₄ are independently hydrogen, alkyl, cycloalkyl, heterocyclyl, halogen, or R₂ and R₃ together with the atom to which they are attached to form a carbocycle or heterocycle, or R₂ and R₄ together with the atom to which they are attached to form a carbocycle or heterocycle.

In some embodiments of the compounds of Formula (I), (I-A), (I-A-I), and (I-B), R₂, R₃, R₄ are independently hydrogen, alkyl, cycloalkyl, heterocyclyl, halogen.

In some embodiments of the compounds of Formula (I), (I-A), (I-A-I), and (I-B), R₂, R₃, R₄ are independently alkyl or hydrogen.

In embodiments of the compounds of Formula (I), (I-A), (I-A-I), and (I-B), R₂ and R₃ are independently hydrogen or alkyl and R₄ is hydrogen.

In embodiments of the compounds of Formula (I), (I-A), (I-A-I), and (I-B), R₂ and R₃ are independently hydrogen or methyl and R₄ is hydrogen.

In some embodiments of the compounds of Formula (I), (I-A), (I-A-I), and (I-B), at least one of R₂, R₃, R₄ is alkyl.

In some embodiments of the compounds of Formula (I), (I-A), (I-A-I), and (I-B), at least two of R₂, R₃, R₄ is alkyl.

In some embodiments of the compounds of Formula (I), (I-A), (I-A-I), and (I-B), R₂, R₃, R₄ are hydrogen.

In some embodiments of the compounds of Formula (I), (I-A), (I-A-I), and (I-B), R₅ and R₆ are each independently hydrogen, alkyl, cycloalkyl, heterocyclyl, alkoxy, —O-cycloalkyl, —O— heterocyclyl, or halogen, or R₅ and R₆ together with the atom to which they are attached to form a carbocycle or heterocycle.

In some embodiments of the compounds of Formula (I), (I-A), (I-A-I), and (I-B), R₅ and R₆ are each independently hydrogen, alkyl, cycloalkyl, heterocyclyl, alkoxy, —O-cycloalkyl, —O— heterocyclyl, or halogen.

In some embodiments of the compounds of Formula (I), (I-A), (I-A-I), and (I-B), R₅ and R₆ are hydrogen.

In some embodiments of the compounds of Formula (I), (I-A), (I-A-I), and (I-B), R₇ and R₈ are independently hydrogen, alkyl, cycloalkyl, or heterocyclyl, or R₇ and R₈ together with the atom to which they are attached form a heterocycle;

In some embodiments of the compounds of Formula (I), (I-A), (I-A-I), and (I-B), R₇ and R₈ are independently hydrogen, alkyl, cycloalkyl, or heterocyclyl.

In some embodiments of the compounds of Formula (I), (I-A), (I-A-I), and (I-B), R₇ and R₈ are alkyl.

In some embodiments of the compounds of Formula (I), (I-A), (I-A-I), and (I-B), R₇ and R₈ are —CH₃.

In some embodiments of the compounds of Formula (I), Y is cycloalkyl, heterocyclyl, heteroaryl or aryl.

In some embodiments of the compounds of Formula (I), Y is cycloalkyl or aryl.

In some embodiments of the compounds of Formula (I), Y is 3-7 membered monocyclic cycloalkyl, 5-8 membered bicyclic cycloalkyl, 4-7 membered saturated heterocyclyl, 5-8 membered bicyclic heterocyclyl, 5-6-membered heteroaryl or phenyl. In embodiments, the 5-10 membered heteroaryl or phenyl is substituted with one or more halogen.

In some embodiments of the compounds of Formula (I), Y is 3-7 membered monocyclic cycloalkyl.

In some embodiments of the compounds of Formula (I), Y is a 6 membered monocyclic cycloalkyl.

In some embodiments of the compounds of Formula (I), Y is an unsubstituted cyclohexyl.

In some embodiments of the compounds of Formula (I), Y is optionally substituted with one or more R^{A} substituents, as defined herein. In some embodiments, Y is optionally substituted with 1, 2, 3, or 4 R^{A}.

In embodiments of the compounds of Formula (I), Y is a 2-oxaspiro[4.5]decane or cyclohexyl optionally substituted one or more alkyl or cycloalkyl.

In embodiments of the compounds of Formula (I), Y is a 2-oxaspiro[4.5]decane or cyclohexyl optionally substituted with C₁₋₃ alkyl or cyclopropyl.

In some embodiments of the compounds of Formula (I), Y is phenyl.

In some embodiments of the compounds of Formula (I), Y is optionally substituted with one or more deuterium.

In some embodiments of the compounds of Formula (I), Z is absent, heteroaryl or aryl,
wherein if L₁ is a bond or CR₅R₆ then Z is heteroaryl or aryl.

In some embodiments of the compounds of Formula (I), Z is heteroaryl or aryl.

In some embodiments of the compounds of Formula (I), Z is heteroaryl.

In some embodiments of the compounds of Formula (I), Z is aryl.

In some embodiments of the compounds of Formula (I), Z is absent.

In some embodiments of the compounds of Formula (I), Z is 5-10 membered heteroaryl or phenyl.

In some embodiments of the compounds of Formula (I), Z is phenyl.

In some embodiments of the compounds of Formula (I), Z is unsubstituted phenyl. In some embodiments Z is phenyl substituted with one or more fluoro. In some embodiments Z is phenyl substituted with one or two halogens.

In embodiments of the compounds of Formula (I), Z is optionally substituted with one or more R^{B} substituents as defined herein. In embodiments, Z is optionally substituted with 1, 2, 3, 4, or 5 R^{B}.

In some embodiments of the compounds of Formula (I), Y and Z are phenyl.

In some embodiments of the compounds of Formula (I), Y is cyclohexyl and Z is phenyl.

In some embodiments of the compounds of Formula (I), Y and Z together are:

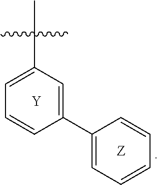

In some embodiments of the compounds of Formula (I), (I-A), (I-A-I), and (I-B), R^{A} and R^{B} are independently, for each occurrence, selected from the group consisting of hydroxy, halo, —NO₂, —CN, —NR₇R₈, —CO₂R₁₀, —OC(O)R₁₀, —COR₁₀, —C(O)NR₇R₈, —NR₇C(O)R₁₀, —OC(O)NR₇R₈, —NR₇C(O)OR₁₀, —S(O), R₁₀ (wherein w is 0, 1, or 2), —OSO₂R₁₀, —SO₃R₁₀, —S(O)₂NR₇R₈, —NR₇S(O)₂R₁₀, —NR₇C(O)NR₇R₈, —C₁₋₆ alkyl-NR₇R₈, —C₁₋₆ alkyl-O—C₁₋₆ alkyl, —C₁₋₆ alkyl, C₁₋₆ alkoxy, C₂₋₆ alkenyl, and C₂₋₆ alkynyl.

In some embodiments of the compounds of Formula (I), (I-A), (I-A-I), and (I-B), R^{B} is halogen.

In some embodiments, of the compounds of Formula (I), (I-A), (I-A-I), and (I-B) R^{B} is fluoro.

In some embodiments of the compounds of Formula (I), (I-A), (I-A-I), and (I-B), o and q are each independently 0, 1, 2, 3, 4, or 5.

In some embodiments of the compounds of Formula (I), (I-A), (I-A-I), and (I-B), o is 0 or 1. In some embodiments, o is 0. In some embodiments, o is 1. In some embodiments, o is 2. In some embodiments o is 3. In some embodiments o is 4. In some embodiments o is 5.

In some embodiments of the compounds of Formula (I), (I-A), (I-A-I), and (I-B), o is 1 and R^{B} is fluoro.

In some embodiments of the compounds of Formula (I), (I-A), (I-A-I), and (I-B), q is 0 or 1. In some embodiments, q is 0. In some embodiments, q is 1. In some embodiments, q is 2. In some embodiments q is 3. In some embodiments q is 4. In some embodiments q is 5.

In some embodiments of the compounds of Formula (I), (I-A), (I-A-I), and (I-B), R₁₀ is independently selected, for each occurrence, from the group consisting of hydrogen, C₁₋₆alkyl, C₂₋₆alkenyl, C₂₋₆alkynyl, aryl, cycloalkyl, heterocyclyl, and heteroaryl.

In all embodiments of the disclosure, the compound is not:

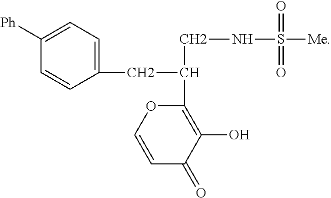 [SMILES: CS(=O)(=O)NCC(Cc1ccc(-c2ccccc2)cc1)c1occc(=O)c1O]

In some embodiments, compounds described herein may also comprise one or more isotopic substitutions. Examples of isotopes suitable for inclusion in the compounds of the present disclosure include isotopes of hydrogen such as ²H and ³H, carbon such as ¹¹C, ¹³C and ¹⁴C, nitrogen such as ¹³N and ¹⁵N, oxygen such as ¹⁸O, phosphorus such as ³²P, sulfur such as ³S, fluorine such as ¹⁸F, iodine such as ¹²³I and ¹²⁵I, and chlorine such as ³⁶Cl. Isotopically enriched compounds of the disclosure can be prepared, for example, by conventional techniques well known to those skilled in the art or by processes analogous to those described in the schemes and examples herein using appropriate isotopically enriched reagents and/or intermediates.

In some embodiments, the compounds disclosed herein are a racemic mixture. In some embodiments, the compounds disclosed herein are enriched in one enantiomer. In some embodiments, the compounds disclosed herein are enriched are substantially free of the opposite enantiomer. In embodiments, provided herein is the (+)-enantiomer of a compound disclosed herein. In embodiments, provided herein is the (−)-enantiomer of a compound disclosed herein. In some embodiments, the compounds disclosed herein have an enantiomeric excess of about or greater than about 55%, about or greater than about 60%, about or greater than about 65%, about or greater than about 70%, about or greater than about 75%, about or greater than about 80%, about or greater than about 85%, about or greater than about 90%, about or greater than about 91%, about or greater than about 92%, about or greater than about 93%, about or greater than about 94%, about or greater than about 95%, about or greater than about 96%, about or greater than about 97%, about or greater than about 98%, about or greater than about 98.5%, about or greater than about 99%, about or greater than about 99.5%, or more, including all subranges and values therebetween. In some embodiments, the compounds of the present disclosure are provided as a mixture of diastereomers. In some embodiments, a diastereomer of a compound of the present disclosure is provided substantially free of other possible diastereomer(s). In embodiments, the present disclosure includes tautomers of any said compounds.

In some embodiments, provided herein is one or more compounds selected from Table 1 or a pharmaceutically acceptable salt thereof, or a stereoisomer thereof.

In some embodiments, provided herein is one or more compounds selected from Table 1 or a pharmaceutically acceptable salt thereof, or an enantiomer thereof.

In some embodiments, provided herein is one or more compounds selected from Table 1 or a pharmaceutically acceptable salt thereof, or a diastereomer, or mixture of diastereomers thereof.

In embodiments, the compound of Formula (I), (I-A), (I-A-I), (I-B), or Table 1 has a (R)-configuration at the carbon labelled with an asterisk (*):

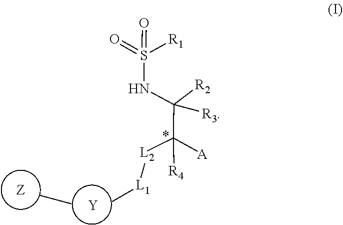

In embodiments, the compound of Formula (I), (I-A), (I-A-I), (I-B), or Table 1 has (S)-configuration at the carbon labelled with an asterisk (*):

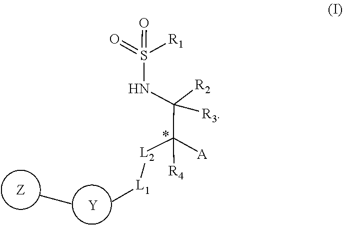

In embodiments, the compound of Formula (I), (I-A), (I-A-I), (I-B), or Table 1 has a (R)-configuration at the carbon labelled with an asterisk (*). In embodiments, the compound of Formula (I), (I-A), (I-A-I), (I-B), or Table 1 has a (S)-configuration at the carbon labelled with an asterisks' (*). In embodiments, the compound of Formula (I), (I-A), (I-A-I), (I-B), or Table 1 a (R)-configuration at the carbon labelled with a double asterisk (**). In embodiments, the compound of Formula (I), (I-A), (I-A-I), (I-B), or Table 1 a (S)-configuration at the carbon labelled with a double asterisk (**):

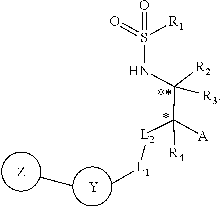

In some embodiments, provided herein is one or more compounds selected from Table 1.

In some embodiments, provided herein is one or more pharmaceutically acceptable salts of a compound selected from Table 1.

TABLE 1
Compounds
Structure

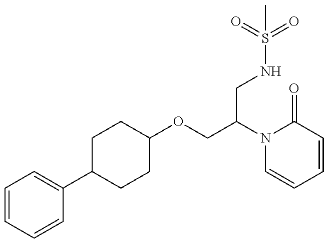 [SMILES: CS(=O)(=O)NCC(COC1CCC(c2ccccc2)CC1)n1ccccc1=O]

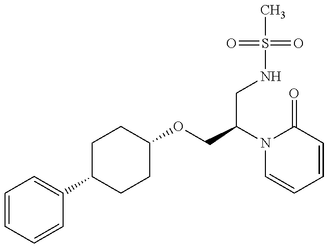 [SMILES: CS(=O)(=O)NC[C@H](CO[C@H]1CC[C@@H](c2ccccc2)CC1)n1ccccc1=O]

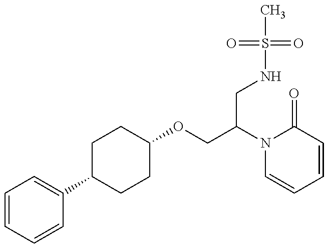 [SMILES: CS(=O)(=O)NCC(CO[C@H]1CC[C@@H](c2ccccc2)CC1)n1ccccc1=O]

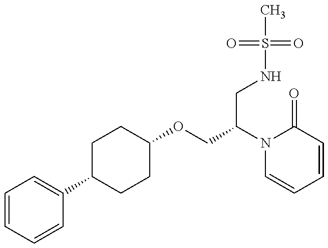 [SMILES: CS(=O)(=O)NC[C@@H](CO[C@H]1CC[C@@H](c2ccccc2)CC1)n1ccccc1=O]

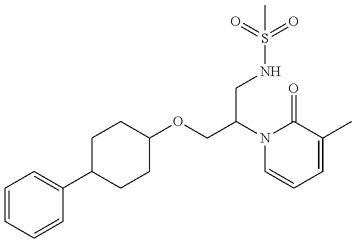 [SMILES: Cc1cccn(C(CNS(C)(=O)=O)COC2CCC(c3ccccc3)CC2)c1=O]

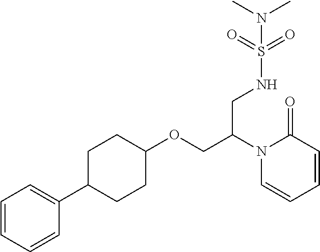 [SMILES: CN(C)S(=O)(=O)NCC(COC1CCC(c2ccccc2)CC1)n1ccccc1=O]

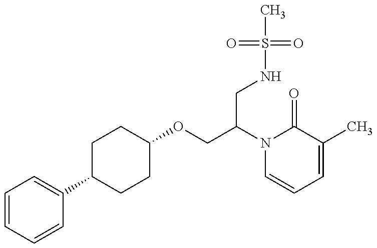 [SMILES: Cc1cccn(C(CNS(C)(=O)=O)CO[C@H]2CC[C@@H](c3ccccc3)CC2)c1=O]

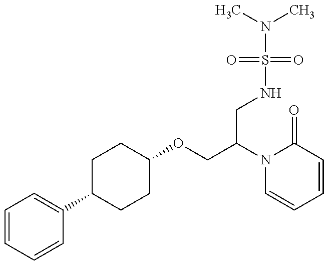 [SMILES: CN(C)S(=O)(=O)NCC(CO[C@H]1CC[C@@H](c2ccccc2)CC1)n1ccccc1=O]

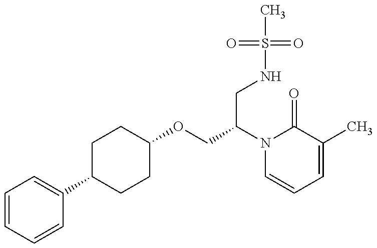 [SMILES: Cc1cccn([C@@H](CNS(C)(=O)=O)CO[C@H]2CC[C@@H](c3ccccc3)CC2)c1=O]

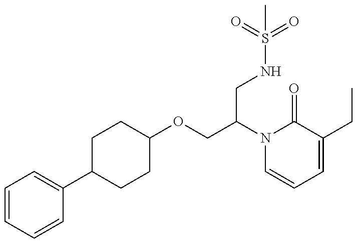 [SMILES: CCc1cccn(C(CNS(C)(=O)=O)COC2CCC(c3ccccc3)CC2)c1=O]

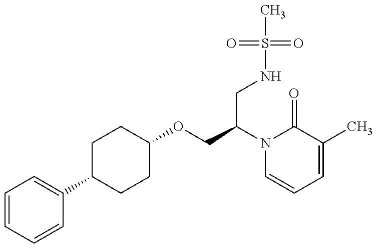 [SMILES: Cc1cccn([C@H](CNS(C)(=O)=O)CO[C@H]2CC[C@@H](c3ccccc3)CC2)c1=O]

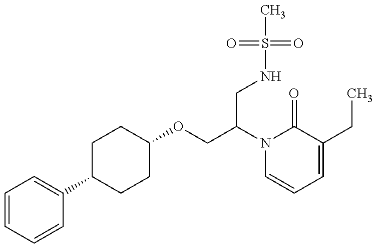 [SMILES: CCc1cccn(C(CNS(C)(=O)=O)CO[C@H]2CC[C@@H](c3ccccc3)CC2)c1=O]

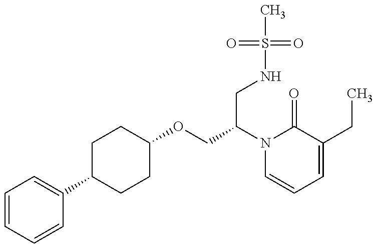 [SMILES: CCc1cccn([C@@H](CNS(C)(=O)=O)CO[C@H]2CC[C@@H](c3ccccc3)CC2)c1=O]

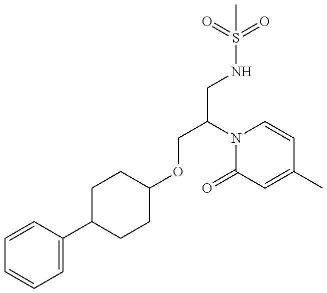 [SMILES: Cc1ccn(C(CNS(C)(=O)=O)COC2CCC(c3ccccc3)CC2)c(=O)c1]

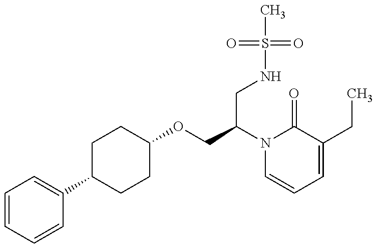 [SMILES: CCc1cccn([C@H](CNS(C)(=O)=O)CO[C@H]2CC[C@@H](c3ccccc3)CC2)c1=O]

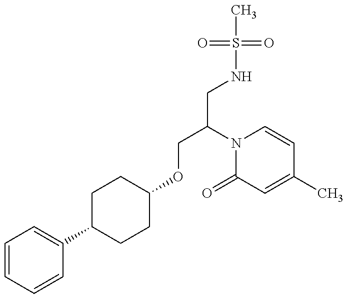 [SMILES: Cc1ccn(C(CNS(C)(=O)=O)CO[C@H]2CC[C@@H](c3ccccc3)CC2)c(=O)c1]

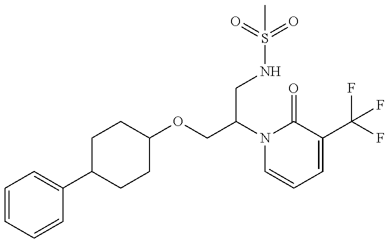 [SMILES: CS(=O)(=O)NCC(COC1CCC(c2ccccc2)CC1)n1cccc(C(F)(F)F)c1=O]

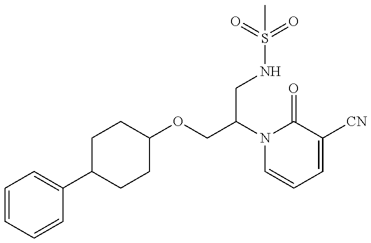 [SMILES: CS(=O)(=O)NCC(COC1CCC(c2ccccc2)CC1)n1cccc(C#N)c1=O]

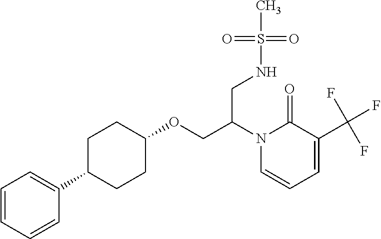 [SMILES: CS(=O)(=O)NCC(CO[C@H]1CC[C@@H](c2ccccc2)CC1)n1cccc(C(F)(F)F)c1=O]

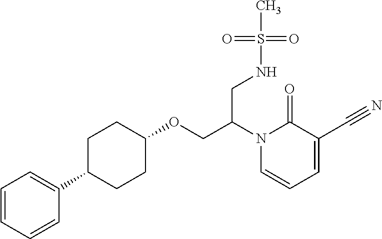 [SMILES: CS(=O)(=O)NCC(CO[C@H]1CC[C@@H](c2ccccc2)CC1)n1cccc(C#N)c1=O]

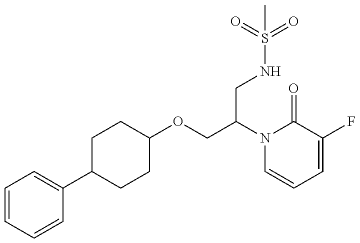 [SMILES: CS(=O)(=O)NCC(COC1CCC(c2ccccc2)CC1)n1cccc(F)c1=O]

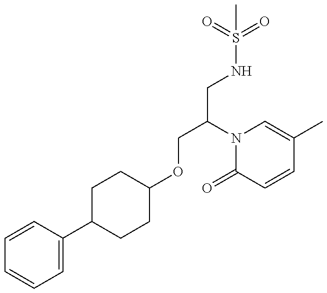 [SMILES: Cc1ccc(=O)n(C(CNS(C)(=O)=O)COC2CCC(c3ccccc3)CC2)c1]

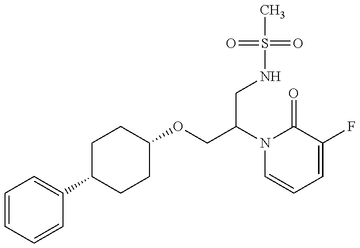 [SMILES: CS(=O)(=O)NCC(CO[C@H]1CC[C@@H](c2ccccc2)CC1)n1cccc(F)c1=O]

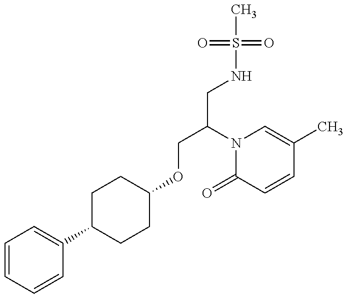 [SMILES: Cc1ccc(=O)n(C(CNS(C)(=O)=O)CO[C@H]2CC[C@@H](c3ccccc3)CC2)c1]

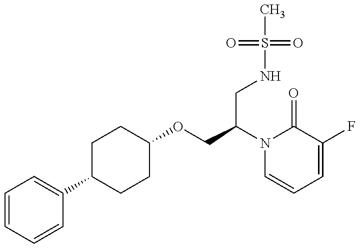 [SMILES: CS(=O)(=O)NC[C@H](CO[C@H]1CC[C@@H](c2ccccc2)CC1)n1cccc(F)c1=O]

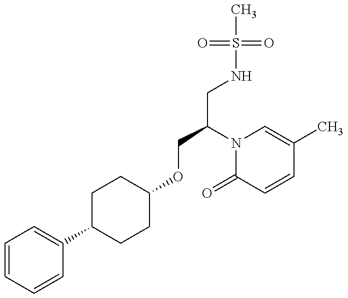 [SMILES: Cc1ccc(=O)n([C@H](CNS(C)(=O)=O)CO[C@H]2CC[C@@H](c3ccccc3)CC2)c1]

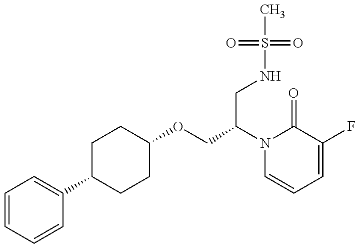 [SMILES: CS(=O)(=O)NC[C@@H](CO[C@H]1CC[C@@H](c2ccccc2)CC1)n1cccc(F)c1=O]

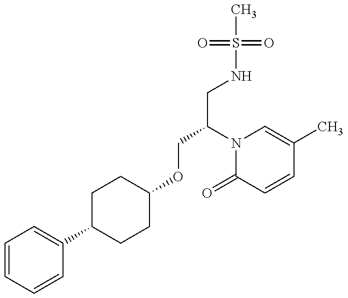 [SMILES: Cc1ccc(=O)n([C@@H](CNS(C)(=O)=O)CO[C@H]2CC[C@@H](c3ccccc3)CC2)c1]

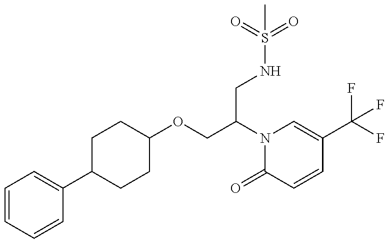 [SMILES: CS(=O)(=O)NCC(COC1CCC(c2ccccc2)CC1)n1cc(C(F)(F)F)ccc1=O]

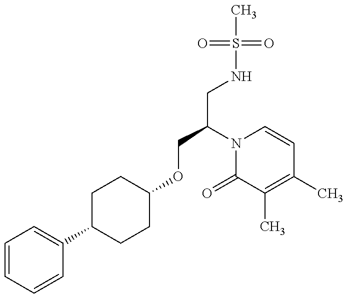 [SMILES: Cc1ccn([C@H](CNS(C)(=O)=O)CO[C@H]2CC[C@@H](c3ccccc3)CC2)c(=O)c1C]

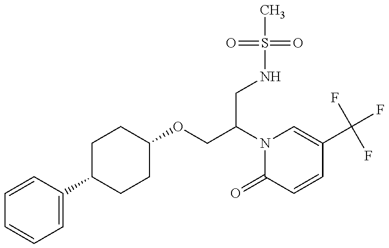 [SMILES: CS(=O)(=O)NCC(CO[C@H]1CC[C@@H](c2ccccc2)CC1)n1cc(C(F)(F)F)ccc1=O]

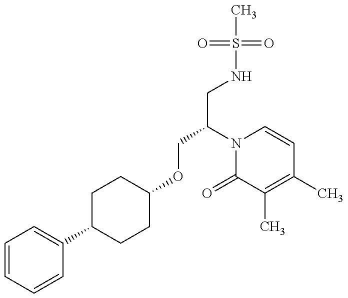 [SMILES: Cc1ccn([C@@H](CNS(C)(=O)=O)CO[C@H]2CC[C@@H](c3ccccc3)CC2)c(=O)c1C]

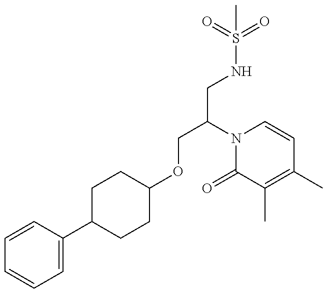 [SMILES: Cc1ccn(C(CNS(C)(=O)=O)COC2CCC(c3ccccc3)CC2)c(=O)c1C]

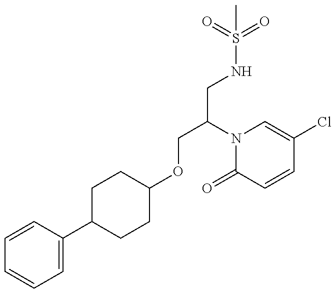 [SMILES: CS(=O)(=O)NCC(COC1CCC(c2ccccc2)CC1)n1cc(Cl)ccc1=O]

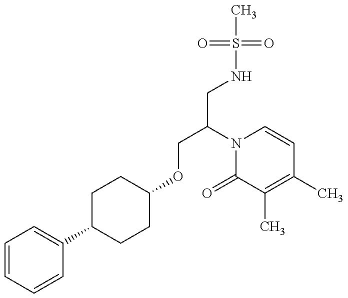 [SMILES: Cc1ccn(C(CNS(C)(=O)=O)CO[C@H]2CC[C@@H](c3ccccc3)CC2)c(=O)c1C]

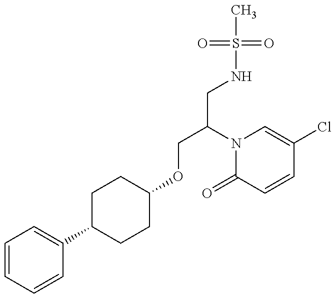 [SMILES: CS(=O)(=O)NCC(CO[C@H]1CC[C@@H](c2ccccc2)CC1)n1cc(Cl)ccc1=O]

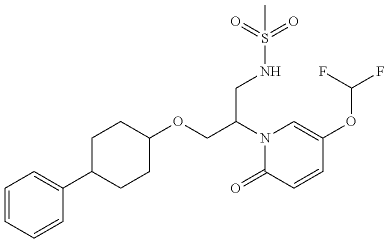 [SMILES: CS(=O)(=O)NCC(COC1CCC(c2ccccc2)CC1)n1cc(OC(F)F)ccc1=O]

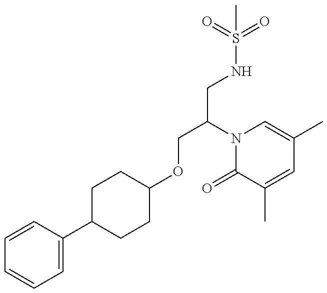 [SMILES: Cc1cc(C)c(=O)n(C(CNS(C)(=O)=O)COC2CCC(c3ccccc3)CC2)c1]

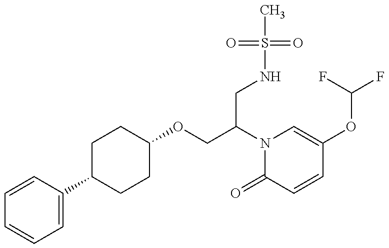 [SMILES: CS(=O)(=O)NCC(CO[C@H]1CC[C@@H](c2ccccc2)CC1)n1cc(OC(F)F)ccc1=O]

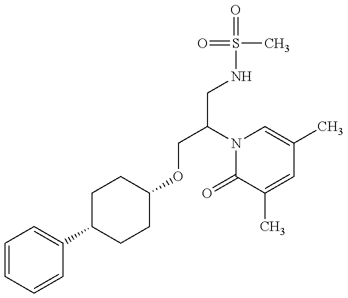 [SMILES: Cc1cc(C)c(=O)n(C(CNS(C)(=O)=O)CO[C@H]2CC[C@@H](c3ccccc3)CC2)c1]

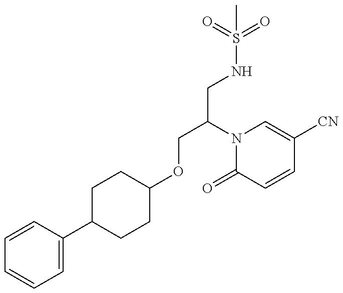 [SMILES: CS(=O)(=O)NCC(COC1CCC(c2ccccc2)CC1)n1cc(C#N)ccc1=O]

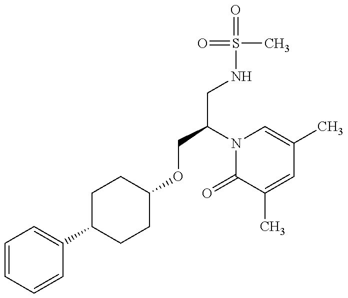 [SMILES: Cc1cc(C)c(=O)n([C@H](CNS(C)(=O)=O)CO[C@H]2CC[C@@H](c3ccccc3)CC2)c1]

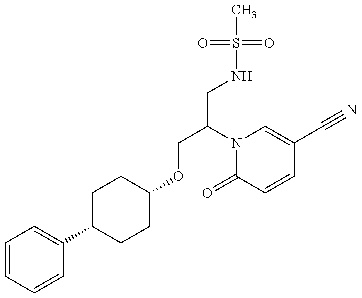 [SMILES: CS(=O)(=O)NCC(CO[C@H]1CC[C@@H](c2ccccc2)CC1)n1cc(C#N)ccc1=O]

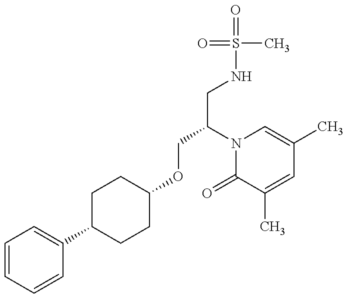 [SMILES: Cc1cc(C)c(=O)n([C@@H](CNS(C)(=O)=O)CO[C@H]2CC[C@@H](c3ccccc3)CC2)c1]

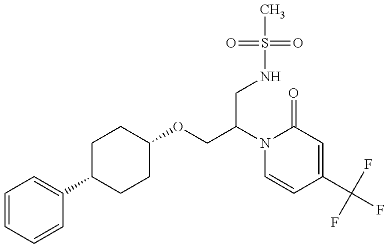 [SMILES: CS(=O)(=O)NCC(CO[C@H]1CC[C@@H](c2ccccc2)CC1)n1ccc(C(F)(F)F)cc1=O]

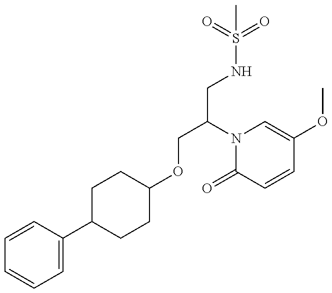 [SMILES: COc1ccc(=O)n(C(CNS(C)(=O)=O)COC2CCC(c3ccccc3)CC2)c1]

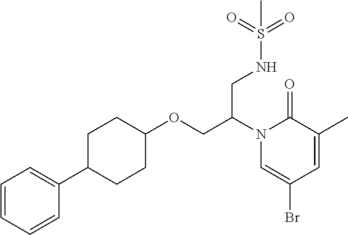 [SMILES: Cc1cc(Br)cn(C(CNS(C)(=O)=O)COC2CCC(c3ccccc3)CC2)c1=O]

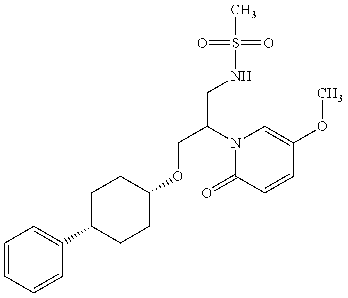 [SMILES: COc1ccc(=O)n(C(CNS(C)(=O)=O)CO[C@H]2CC[C@@H](c3ccccc3)CC2)c1]

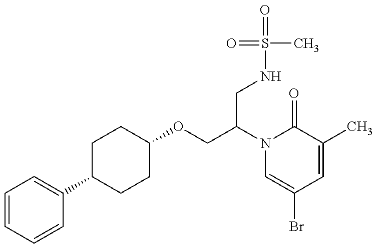 [SMILES: Cc1cc(Br)cn(C(CNS(C)(=O)=O)CO[C@H]2CC[C@@H](c3ccccc3)CC2)c1=O]

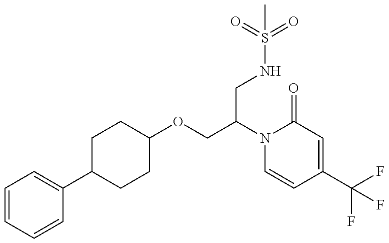 [SMILES: CS(=O)(=O)NCC(COC1CCC(c2ccccc2)CC1)n1ccc(C(F)(F)F)cc1=O]

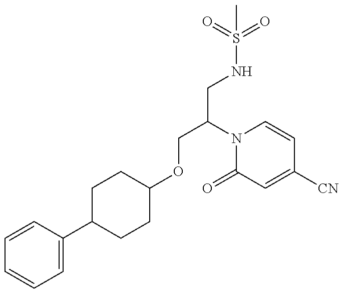 [SMILES: CS(=O)(=O)NCC(COC1CCC(c2ccccc2)CC1)n1ccc(C#N)cc1=O]

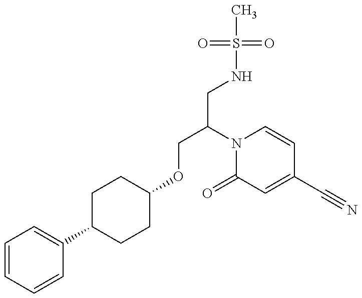 [SMILES: CS(=O)(=O)NCC(CO[C@H]1CC[C@@H](c2ccccc2)CC1)n1ccc(C#N)cc1=O]

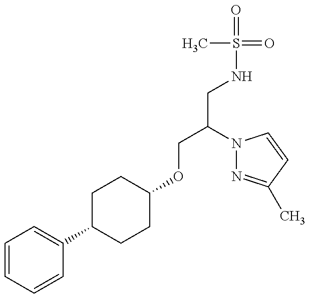 [SMILES: Cc1ccn(C(CNS(C)(=O)=O)CO[C@H]2CC[C@@H](c3ccccc3)CC2)n1]

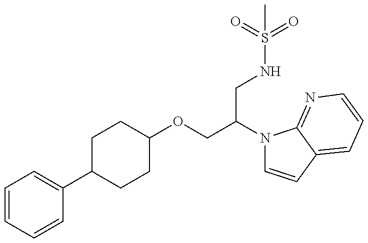 [SMILES: CS(=O)(=O)NCC(COC1CCC(c2ccccc2)CC1)n1ccc2cccnc21]

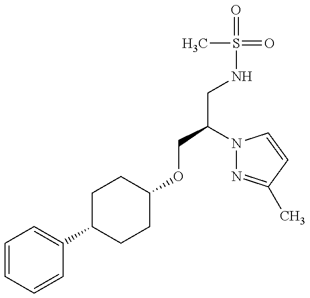 [SMILES: Cc1ccn([C@H](CNS(C)(=O)=O)CO[C@H]2CC[C@@H](c3ccccc3)CC2)n1]

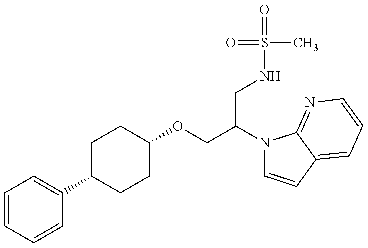 [SMILES: CS(=O)(=O)NCC(CO[C@H]1CC[C@@H](c2ccccc2)CC1)n1ccc2cccnc21]

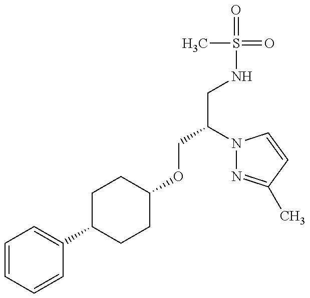 [SMILES: Cc1ccn([C@@H](CNS(C)(=O)=O)CO[C@H]2CC[C@@H](c3ccccc3)CC2)n1]

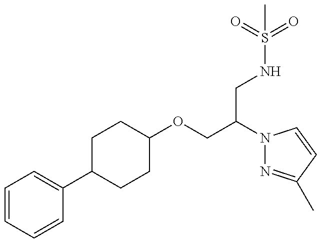 [SMILES: Cc1ccn(C(CNS(C)(=O)=O)COC2CCC(c3ccccc3)CC2)n1]

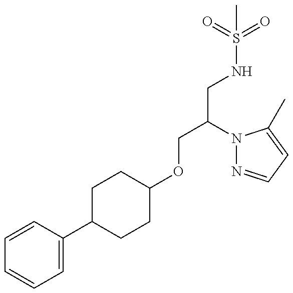 [SMILES: Cc1ccnn1C(CNS(C)(=O)=O)COC1CCC(c2ccccc2)CC1]

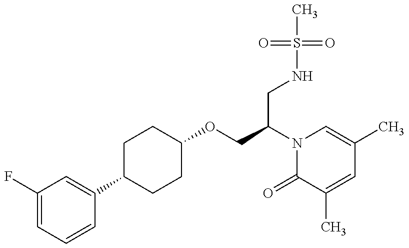 [SMILES: Cc1cc(C)c(=O)n([C@H](CNS(C)(=O)=O)CO[C@H]2CC[C@@H](c3cccc(F)c3)CC2)c1]

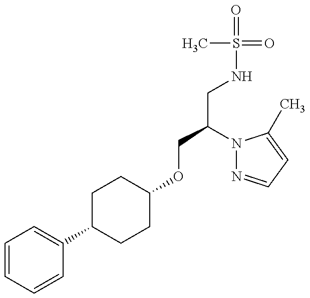 [SMILES: Cc1ccnn1[C@H](CNS(C)(=O)=O)CO[C@H]1CC[C@@H](c2ccccc2)CC1]

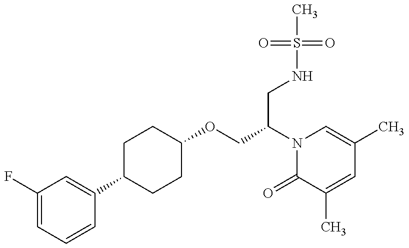 [SMILES: Cc1cc(C)c(=O)n([C@@H](CNS(C)(=O)=O)CO[C@H]2CC[C@@H](c3cccc(F)c3)CC2)c1]

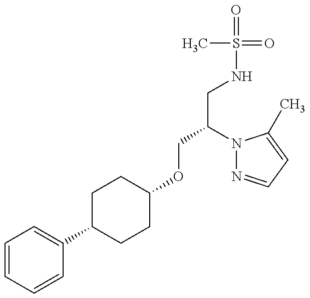 [SMILES: Cc1ccnn1[C@@H](CNS(C)(=O)=O)CO[C@H]1CC[C@@H](c2ccccc2)CC1]

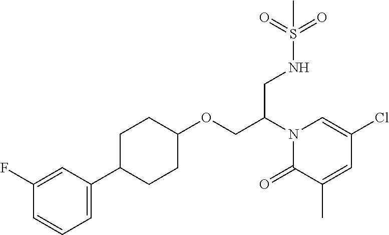 [SMILES: Cc1cc(Cl)cn(C(CNS(C)(=O)=O)COC2CCC(c3cccc(F)c3)CC2)c1=O]

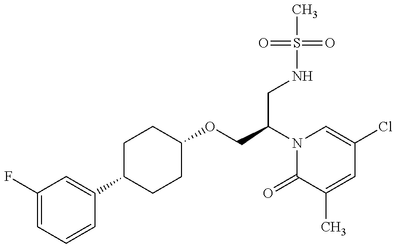 [SMILES: Cc1cc(Cl)cn([C@H](CNS(C)(=O)=O)CO[C@H]2CC[C@@H](c3cccc(F)c3)CC2)c1=O]

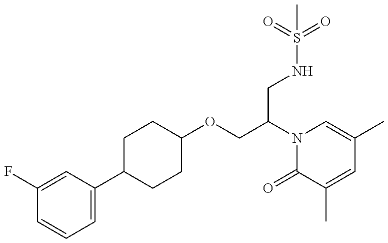 [SMILES: Cc1cc(C)c(=O)n(C(CNS(C)(=O)=O)COC2CCC(c3cccc(F)c3)CC2)c1]

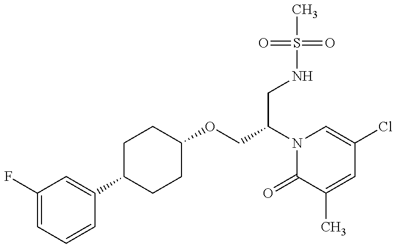 [SMILES: Cc1cc(Cl)cn([C@@H](CNS(C)(=O)=O)CO[C@H]2CC[C@@H](c3cccc(F)c3)CC2)c1=O]

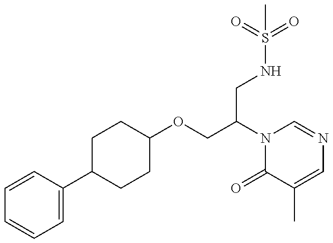 [SMILES: Cc1cncn(C(CNS(C)(=O)=O)COC2CCC(c3ccccc3)CC2)c1=O]

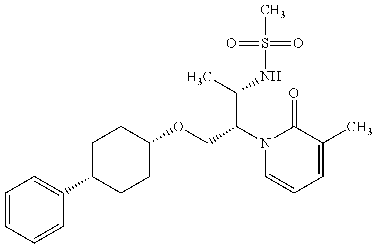 [SMILES: Cc1cccn([C@H](CO[C@H]2CC[C@@H](c3ccccc3)CC2)[C@H](C)NS(C)(=O)=O)c1=O]

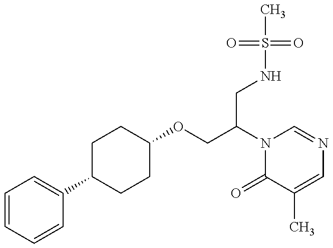 [SMILES: Cc1cncn(C(CNS(C)(=O)=O)CO[C@H]2CC[C@@H](c3ccccc3)CC2)c1=O]

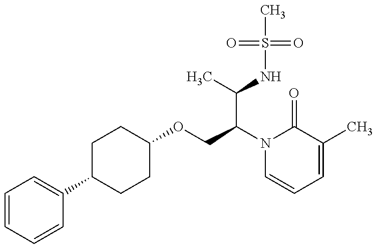 [SMILES: Cc1cccn([C@@H](CO[C@H]2CC[C@@H](c3ccccc3)CC2)[C@@H](C)NS(C)(=O)=O)c1=O]

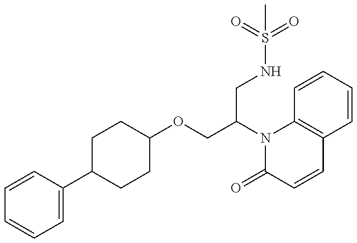 [SMILES: CS(=O)(=O)NCC(COC1CCC(c2ccccc2)CC1)n1c(=O)ccc2ccccc21]

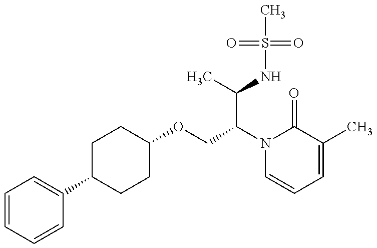 [SMILES: Cc1cccn([C@H](CO[C@H]2CC[C@@H](c3ccccc3)CC2)[C@@H](C)NS(C)(=O)=O)c1=O]

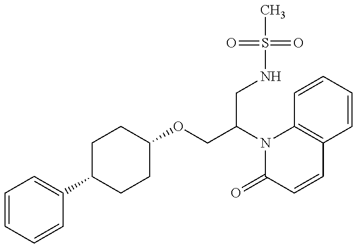 [SMILES: CS(=O)(=O)NCC(CO[C@H]1CC[C@@H](c2ccccc2)CC1)n1c(=O)ccc2ccccc21]

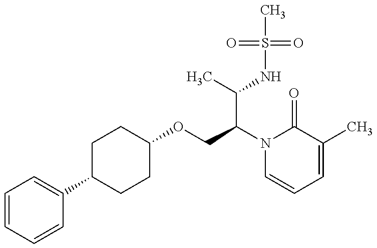 [SMILES: Cc1cccn([C@@H](CO[C@H]2CC[C@@H](c3ccccc3)CC2)[C@H](C)NS(C)(=O)=O)c1=O]

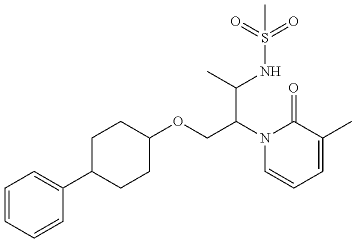 [SMILES: Cc1cccn(C(COC2CCC(c3ccccc3)CC2)C(C)NS(C)(=O)=O)c1=O]

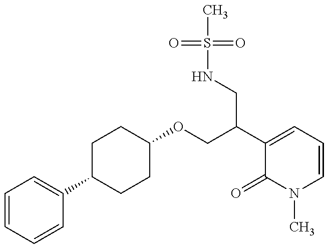 [SMILES: Cn1cccc(C(CNS(C)(=O)=O)CO[C@H]2CC[C@@H](c3ccccc3)CC2)c1=O]

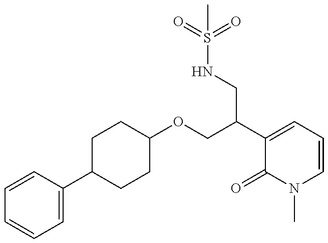 [SMILES: Cn1cccc(C(CNS(C)(=O)=O)COC2CCC(c3ccccc3)CC2)c1=O]

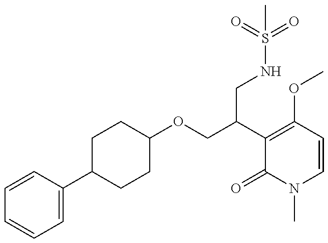 [SMILES: COc1ccn(C)c(=O)c1C(CNS(C)(=O)=O)COC1CCC(c2ccccc2)CC1]

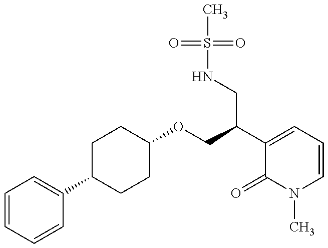 [SMILES: Cn1cccc([C@H](CNS(C)(=O)=O)CO[C@H]2CC[C@@H](c3ccccc3)CC2)c1=O]

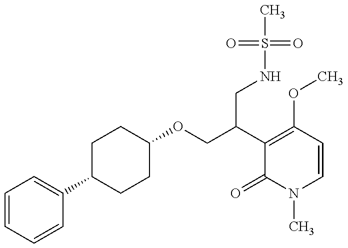 [SMILES: COc1ccn(C)c(=O)c1C(CNS(C)(=O)=O)CO[C@H]1CC[C@@H](c2ccccc2)CC1]

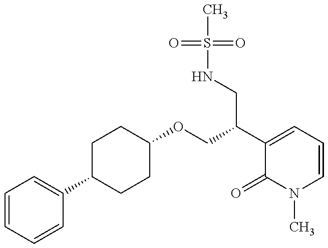 [SMILES: Cn1cccc([C@@H](CNS(C)(=O)=O)CO[C@H]2CC[C@@H](c3ccccc3)CC2)c1=O]

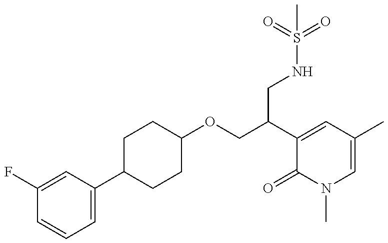 [SMILES: Cc1cc(C(CNS(C)(=O)=O)COC2CCC(c3cccc(F)c3)CC2)c(=O)n(C)c1]

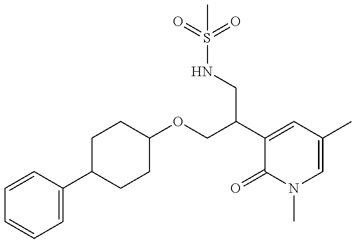 [SMILES: Cc1cc(C(CNS(C)(=O)=O)COC2CCC(c3ccccc3)CC2)c(=O)n(C)c1]

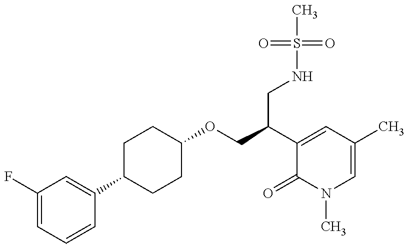 [SMILES: Cc1cc([C@H](CNS(C)(=O)=O)CO[C@H]2CC[C@@H](c3cccc(F)c3)CC2)c(=O)n(C)c1]

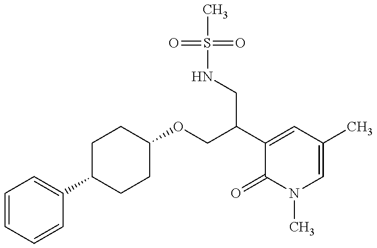 [SMILES: Cc1cc(C(CNS(C)(=O)=O)CO[C@H]2CC[C@@H](c3ccccc3)CC2)c(=O)n(C)c1]

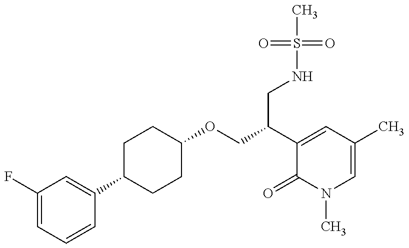 [SMILES: Cc1cc([C@@H](CNS(C)(=O)=O)CO[C@H]2CC[C@@H](c3cccc(F)c3)CC2)c(=O)n(C)c1]

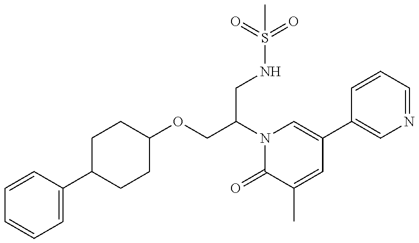 [SMILES: Cc1cc(-c2cccnc2)cn(C(CNS(C)(=O)=O)COC2CCC(c3ccccc3)CC2)c1=O]

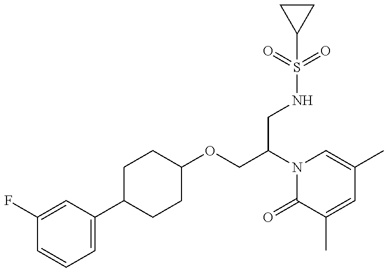 [SMILES: Cc1cc(C)c(=O)n(C(CNS(=O)(=O)C2CC2)COC2CCC(c3cccc(F)c3)CC2)c1]

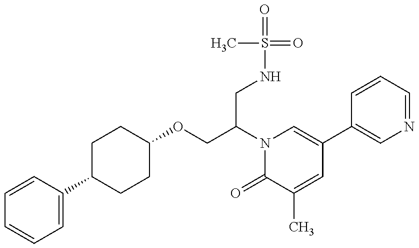 [SMILES: Cc1cc(-c2cccnc2)cn(C(CNS(C)(=O)=O)CO[C@H]2CC[C@@H](c3ccccc3)CC2)c1=O]

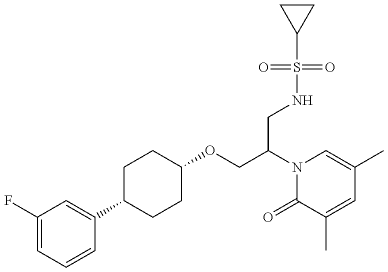 [SMILES: Cc1cc(C)c(=O)n(C(CNS(=O)(=O)C2CC2)CO[C@H]2CC[C@@H](c3cccc(F)c3)CC2)c1]

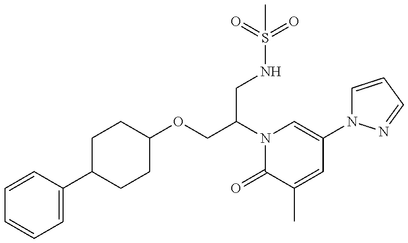 [SMILES: Cc1cc(-n2cccn2)cn(C(CNS(C)(=O)=O)COC2CCC(c3ccccc3)CC2)c1=O]

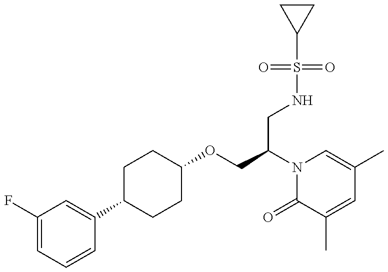 [SMILES: Cc1cc(C)c(=O)n([C@H](CNS(=O)(=O)C2CC2)CO[C@H]2CC[C@@H](c3cccc(F)c3)CC2)c1]

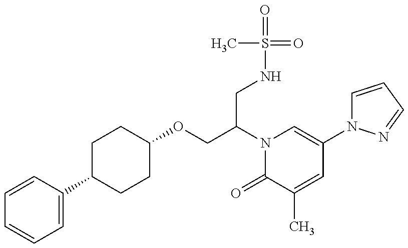 [SMILES: Cc1cc(-n2cccn2)cn(C(CNS(C)(=O)=O)CO[C@H]2CC[C@@H](c3ccccc3)CC2)c1=O]

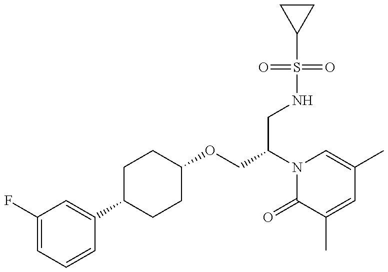 [SMILES: Cc1cc(C)c(=O)n([C@@H](CNS(=O)(=O)C2CC2)CO[C@H]2CC[C@@H](c3cccc(F)c3)CC2)c1]

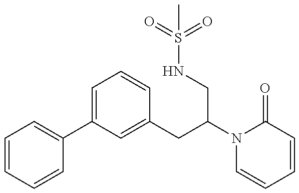 [SMILES: CS(=O)(=O)NCC(Cc1cccc(-c2ccccc2)c1)n1ccccc1=O]

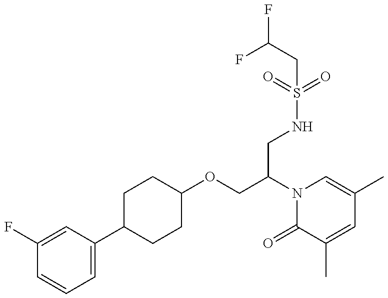 [SMILES: Cc1cc(C)c(=O)n(C(CNS(=O)(=O)CC(F)F)COC2CCC(c3cccc(F)c3)CC2)c1]

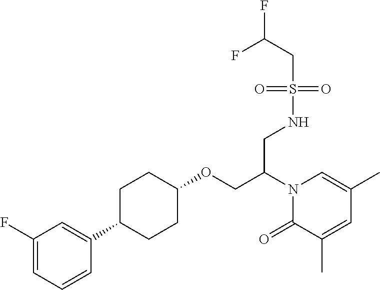 [SMILES: Cc1cc(C)c(=O)n(C(CNS(=O)(=O)CC(F)F)CO[C@H]2CC[C@@H](c3cccc(F)c3)CC2)c1]

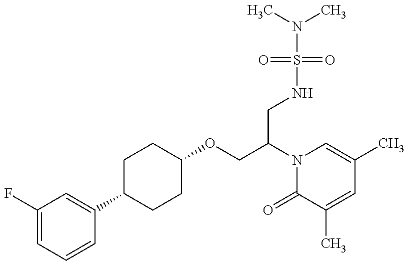 [SMILES: Cc1cc(C)c(=O)n(C(CNS(=O)(=O)N(C)C)CO[C@H]2CC[C@@H](c3cccc(F)c3)CC2)c1]

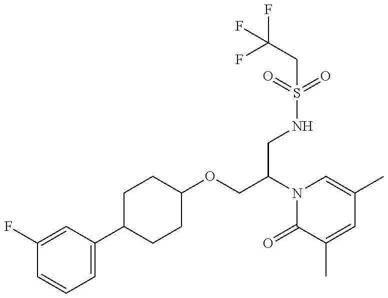 [SMILES: Cc1cc(C)c(=O)n(C(CNS(=O)(=O)CC(F)(F)F)COC2CCC(c3cccc(F)c3)CC2)c1]

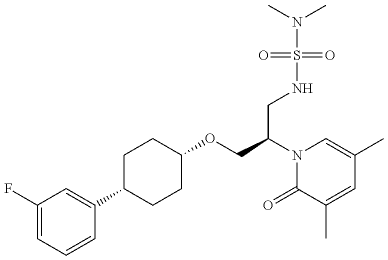 [SMILES: Cc1cc(C)c(=O)n([C@H](CNS(=O)(=O)N(C)C)CO[C@H]2CC[C@@H](c3cccc(F)c3)CC2)c1]

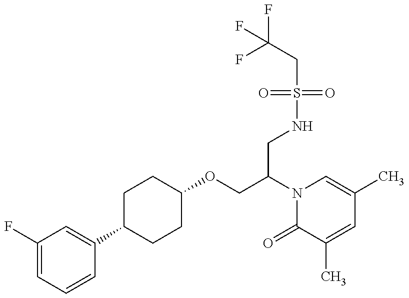 [SMILES: Cc1cc(C)c(=O)n(C(CNS(=O)(=O)CC(F)(F)F)CO[C@H]2CC[C@@H](c3cccc(F)c3)CC2)c1]

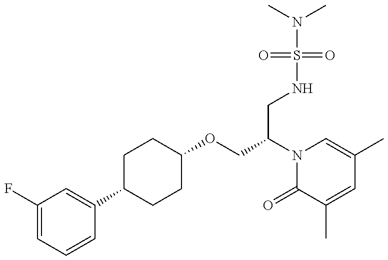 [SMILES: Cc1cc(C)c(=O)n([C@@H](CNS(=O)(=O)N(C)C)CO[C@H]2CC[C@@H](c3cccc(F)c3)CC2)c1]

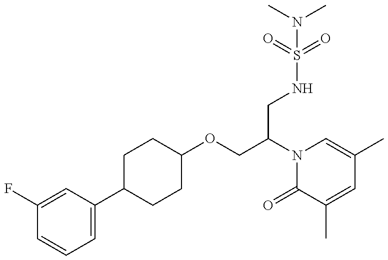 [SMILES: Cc1cc(C)c(=O)n(C(CNS(=O)(=O)N(C)C)COC2CCC(c3cccc(F)c3)CC2)c1]

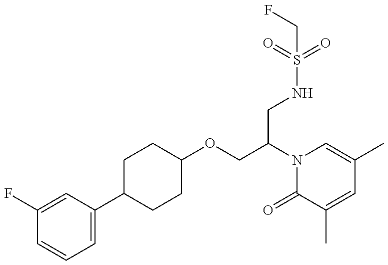 [SMILES: Cc1cc(C)c(=O)n(C(CNS(=O)(=O)CF)COC2CCC(c3cccc(F)c3)CC2)c1]

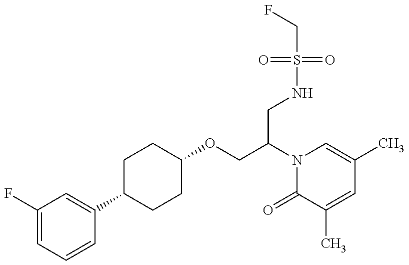 [SMILES: Cc1cc(C)c(=O)n(C(CNS(=O)(=O)CF)CO[C@H]2CC[C@@H](c3cccc(F)c3)CC2)c1]

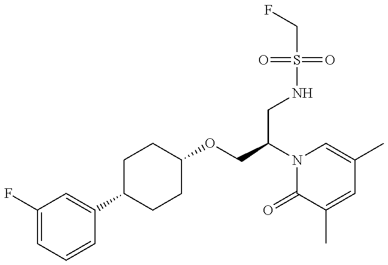 [SMILES: Cc1cc(C)c(=O)n([C@H](CNS(=O)(=O)CF)CO[C@H]2CC[C@@H](c3cccc(F)c3)CC2)c1]

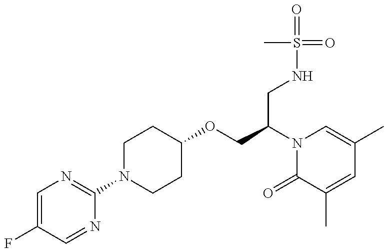

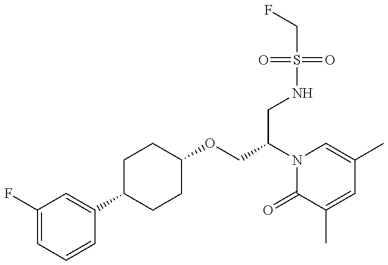 [SMILES: Cc1cc(C)c(=O)n([C@@H](CNS(=O)(=O)CF)CO[C@H]2CC[C@@H](c3cccc(F)c3)CC2)c1]

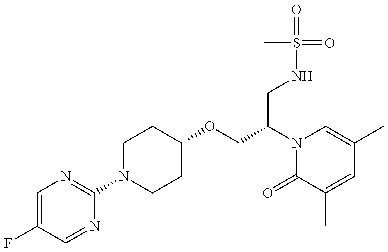

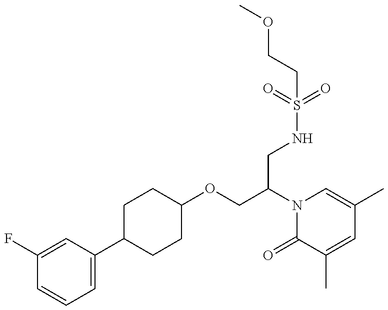 [SMILES: COCCS(=O)(=O)NCC(COC1CCC(c2cccc(F)c2)CC1)n1cc(C)cc(C)c1=O]

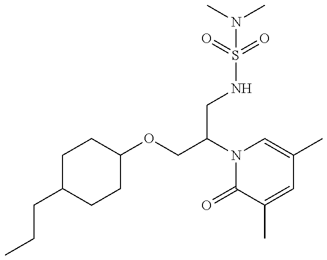 [SMILES: CCCC1CCC(OCC(CNS(=O)(=O)N(C)C)n2cc(C)cc(C)c2=O)CC1]

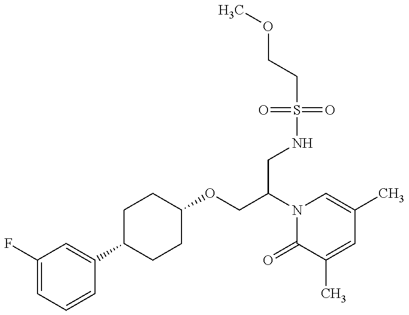 [SMILES: COCCS(=O)(=O)NCC(CO[C@H]1CC[C@@H](c2cccc(F)c2)CC1)n1cc(C)cc(C)c1=O]

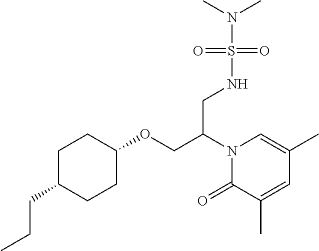 [SMILES: CCC[C@H]1CC[C@@H](OCC(CNS(=O)(=O)N(C)C)n2cc(C)cc(C)c2=O)CC1]

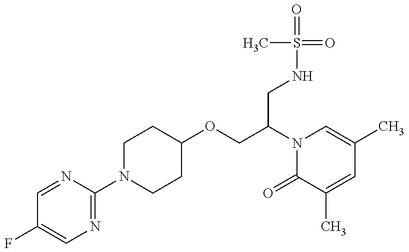 [SMILES: Cc1cc(C)c(=O)n(C(CNS(C)(=O)=O)COC2CCN(c3ncc(F)cn3)CC2)c1]

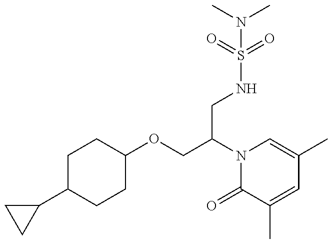 [SMILES: Cc1cc(C)c(=O)n(C(CNS(=O)(=O)N(C)C)COC2CCC(C3CC3)CC2)c1]

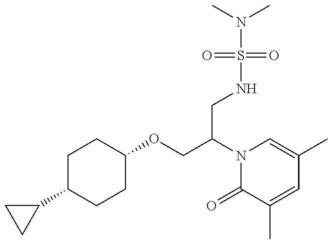 [SMILES: Cc1cc(C)c(=O)n(C(CNS(=O)(=O)N(C)C)CO[C@H]2CC[C@@H](C3CC3)CC2)c1]

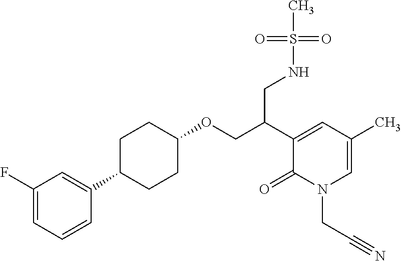 [SMILES: Cc1cc(C(CNS(C)(=O)=O)CO[C@H]2CC[C@@H](c3cccc(F)c3)CC2)c(=O)n(CC#N)c1]

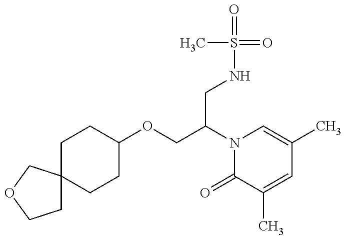 [SMILES: Cc1cc(C)c(=O)n(C(CNS(C)(=O)=O)COC2CCC3(CCOC3)CC2)c1]

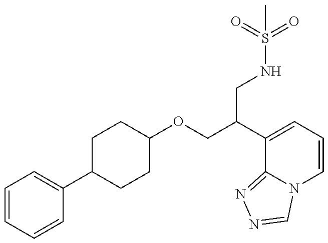 [SMILES: CS(=O)(=O)NCC(COC1CCC(c2ccccc2)CC1)c1cccn2cnnc12]

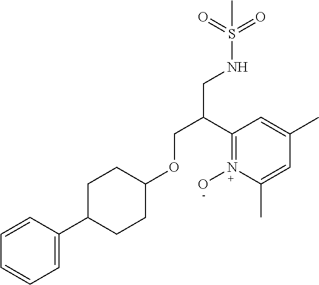

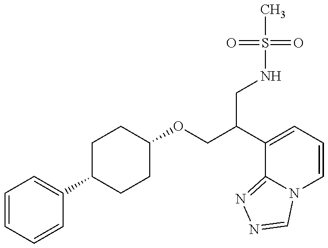 [SMILES: CS(=O)(=O)NCC(CO[C@H]1CC[C@@H](c2ccccc2)CC1)c1cccn2cnnc12]

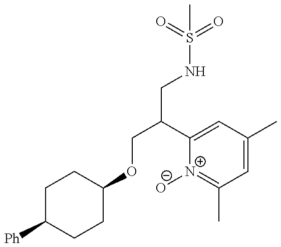 [SMILES: Cc1cc(C)[n+]([O-])c(C(CNS(C)(=O)=O)CO[C@H]2CC[C@@H](c3ccccc3)CC2)c1]

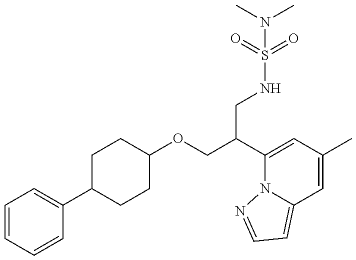 [SMILES: Cc1cc(C(CNS(=O)(=O)N(C)C)COC2CCC(c3ccccc3)CC2)n2nccc2c1]

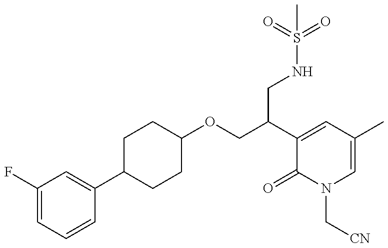 [SMILES: Cc1cc(C(CNS(C)(=O)=O)COC2CCC(c3cccc(F)c3)CC2)c(=O)n(CC#N)c1]

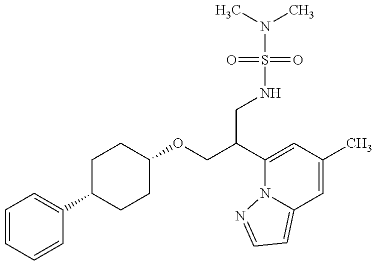 [SMILES: Cc1cc(C(CNS(=O)(=O)N(C)C)CO[C@H]2CC[C@@H](c3ccccc3)CC2)n2nccc2c1]

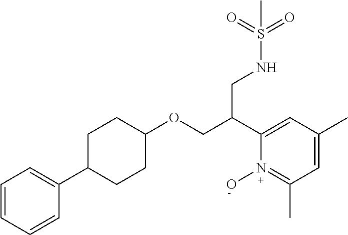

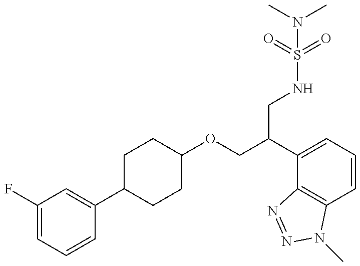 [SMILES: CN(C)S(=O)(=O)NCC(COC1CCC(c2cccc(F)c2)CC1)c1cccc2c1nnn2C]

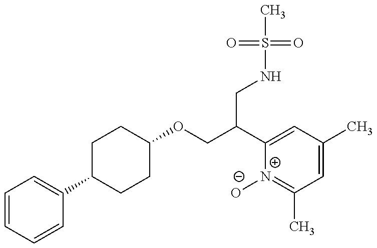 [SMILES: Cc1cc(C)[n+]([O-])c(C(CNS(C)(=O)=O)CO[C@H]2CC[C@@H](c3ccccc3)CC2)c1]

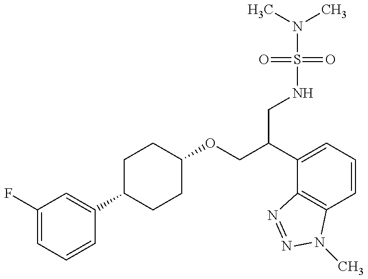 [SMILES: CN(C)S(=O)(=O)NCC(CO[C@H]1CC[C@@H](c2cccc(F)c2)CC1)c1cccc2c1nnn2C]

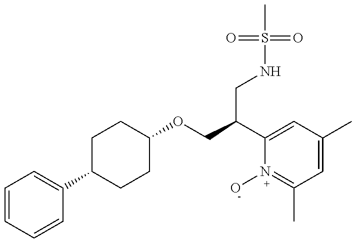

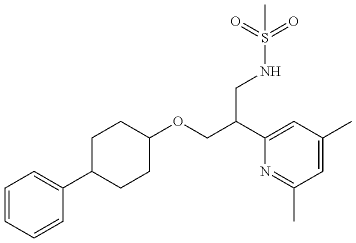 [SMILES: Cc1cc(C)nc(C(CNS(C)(=O)=O)COC2CCC(c3ccccc3)CC2)c1]

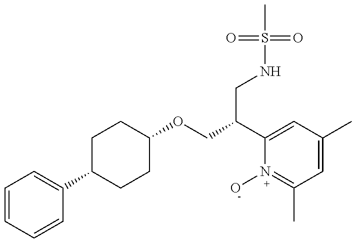

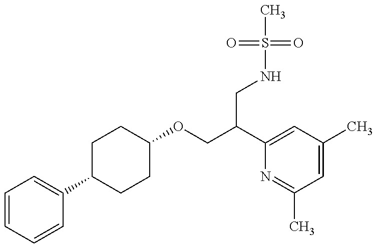 [SMILES: Cc1cc(C)nc(C(CNS(C)(=O)=O)CO[C@H]2CC[C@@H](c3ccccc3)CC2)c1]

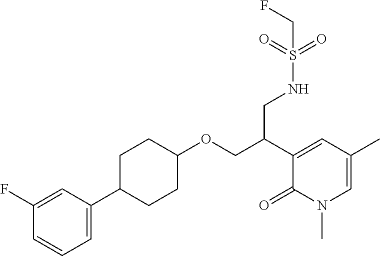 [SMILES: Cc1cc(C(CNS(=O)(=O)CF)COC2CCC(c3cccc(F)c3)CC2)c(=O)n(C)c1]

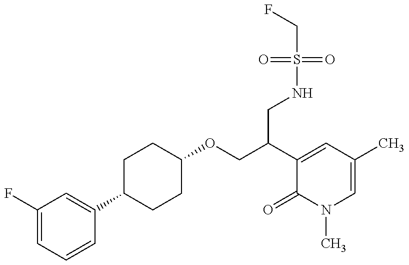 [SMILES: Cc1cc(C(CNS(=O)(=O)CF)CO[C@H]2CC[C@@H](c3cccc(F)c3)CC2)c(=O)n(C)c1]

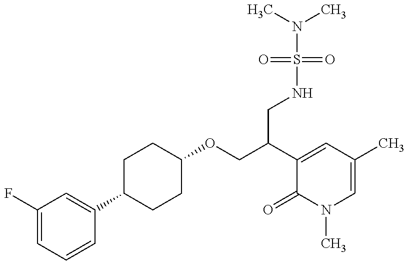 [SMILES: Cc1cc(C(CNS(=O)(=O)N(C)C)CO[C@H]2CC[C@@H](c3cccc(F)c3)CC2)c(=O)n(C)c1]

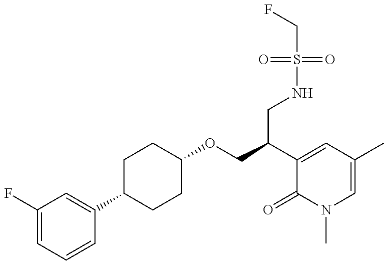 [SMILES: Cc1cc([C@H](CNS(=O)(=O)CF)CO[C@H]2CC[C@@H](c3cccc(F)c3)CC2)c(=O)n(C)c1]

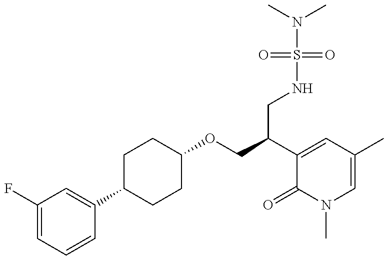 [SMILES: Cc1cc([C@H](CNS(=O)(=O)N(C)C)CO[C@H]2CC[C@@H](c3cccc(F)c3)CC2)c(=O)n(C)c1]

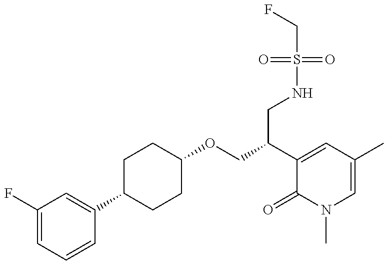 [SMILES: Cc1cc([C@@H](CNS(=O)(=O)CF)CO[C@H]2CC[C@@H](c3cccc(F)c3)CC2)c(=O)n(C)c1]

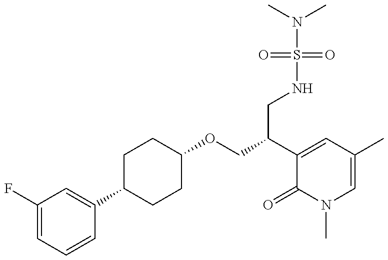 [SMILES: Cc1cc([C@@H](CNS(=O)(=O)N(C)C)CO[C@H]2CC[C@@H](c3cccc(F)c3)CC2)c(=O)n(C)c1]

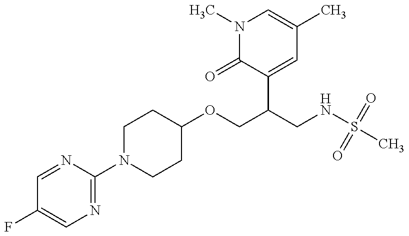 [SMILES: Cc1cc(C(CNS(C)(=O)=O)COC2CCN(c3ncc(F)cn3)CC2)c(=O)n(C)c1]

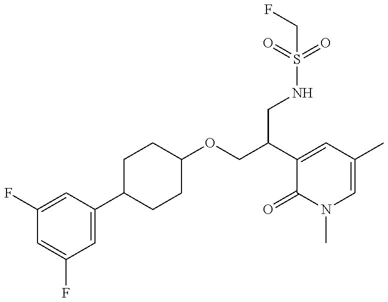 [SMILES: Cc1cc(C(CNS(=O)(=O)CF)COC2CCC(c3cc(F)cc(F)c3)CC2)c(=O)n(C)c1]

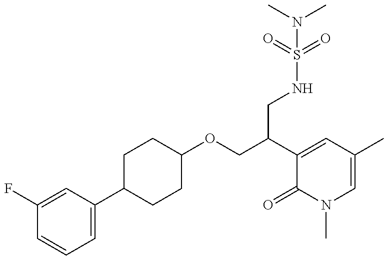 [SMILES: Cc1cc(C(CNS(=O)(=O)N(C)C)COC2CCC(c3cccc(F)c3)CC2)c(=O)n(C)c1]

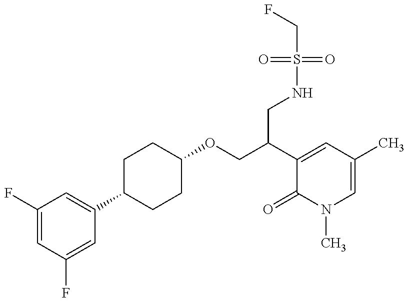 [SMILES: Cc1cc(C(CNS(=O)(=O)CF)CO[C@H]2CC[C@@H](c3cc(F)cc(F)c3)CC2)c(=O)n(C)c1]

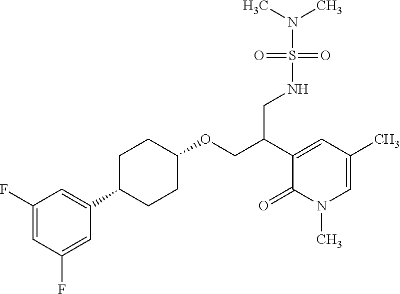 [SMILES: Cc1cc(C(CNS(=O)(=O)N(C)C)CO[C@H]2CC[C@@H](c3cc(F)cc(F)c3)CC2)c(=O)n(C)c1]

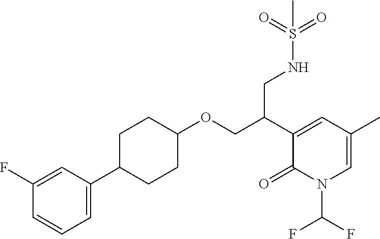 [SMILES: Cc1cc(C(CNS(C)(=O)=O)COC2CCC(c3cccc(F)c3)CC2)c(=O)n(C(F)F)c1]

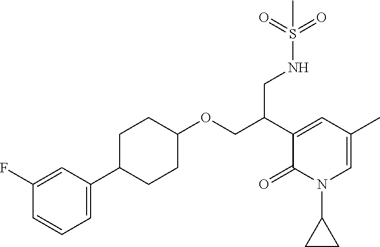 [SMILES: Cc1cc(C(CNS(C)(=O)=O)COC2CCC(c3cccc(F)c3)CC2)c(=O)n(C2CC2)c1]

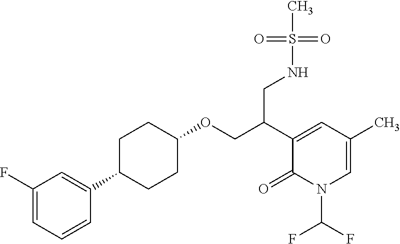 [SMILES: Cc1cc(C(CNS(C)(=O)=O)CO[C@H]2CC[C@@H](c3cccc(F)c3)CC2)c(=O)n(C(F)F)c1]

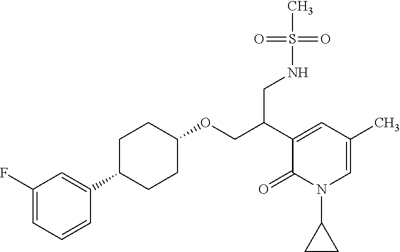 [SMILES: Cc1cc(C(CNS(C)(=O)=O)CO[C@H]2CC[C@@H](c3cccc(F)c3)CC2)c(=O)n(C2CC2)c1]

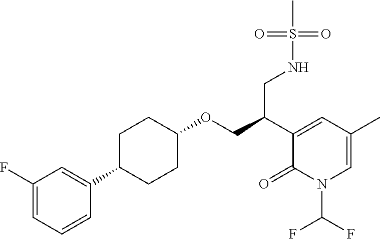 [SMILES: Cc1cc([C@H](CNS(C)(=O)=O)CO[C@H]2CC[C@@H](c3cccc(F)c3)CC2)c(=O)n(C(F)F)c1]

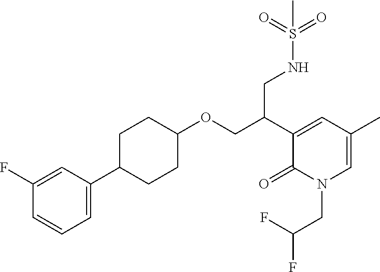 [SMILES: Cc1cc(C(CNS(C)(=O)=O)COC2CCC(c3cccc(F)c3)CC2)c(=O)n(CC(F)F)c1]

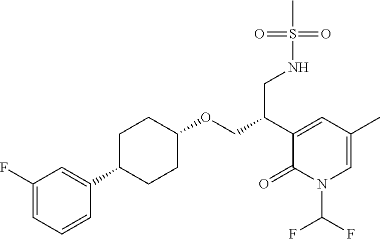 [SMILES: Cc1cc([C@@H](CNS(C)(=O)=O)CO[C@H]2CC[C@@H](c3cccc(F)c3)CC2)c(=O)n(C(F)F)c1]

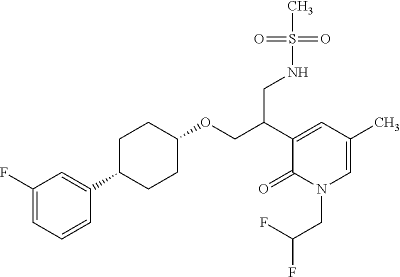 [SMILES: Cc1cc(C(CNS(C)(=O)=O)CO[C@H]2CC[C@@H](c3cccc(F)c3)CC2)c(=O)n(CC(F)F)c1]

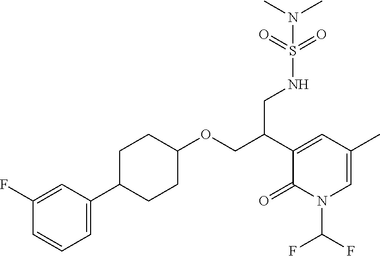 [SMILES: Cc1cc(C(CNS(=O)(=O)N(C)C)COC2CCC(c3cccc(F)c3)CC2)c(=O)n(C(F)F)c1]

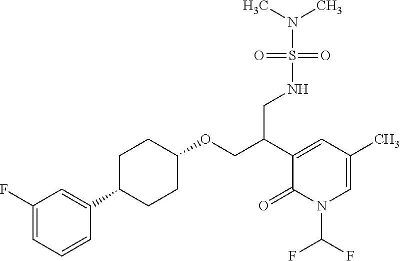 [SMILES: Cc1cc(C(CNS(=O)(=O)N(C)C)CO[C@H]2CC[C@@H](c3cccc(F)c3)CC2)c(=O)n(C(F)F)c1]

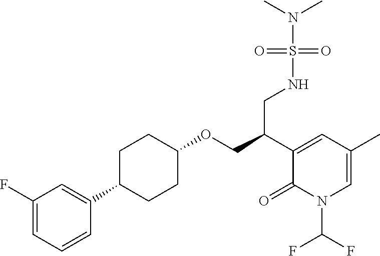 [SMILES: Cc1cc([C@H](CNS(=O)(=O)N(C)C)CO[C@H]2CC[C@@H](c3cccc(F)c3)CC2)c(=O)n(C(F)F)c1]

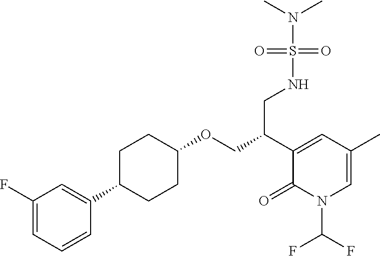 [SMILES: Cc1cc([C@@H](CNS(=O)(=O)N(C)C)CO[C@H]2CC[C@@H](c3cccc(F)c3)CC2)c(=O)n(C(F)F)c1]

### Compositions

The present disclosure provides pharmaceutical compositions for modulating orexin receptor (e.g., orexin type 2 receptor) in a subject. In some embodiments, a pharmaceutical composition comprises one or more compounds of the present disclosure (e.g., a compound of Formula (I), (I-A), (I-A-I), (I-B), or Table 1) or a pharmaceutically acceptable salt thereof. In some embodiments, a pharmaceutical composition comprises one or more compounds of the present disclosure (e.g., a compound of Formula (I), (I-A), (I-A-I), (I-B), or Table 1) or a pharmaceutically acceptable salt thereof or N-oxide thereof.

In some embodiments of the present disclosure, a pharmaceutical composition comprises a therapeutically effective amounts of one or more compounds of the present disclosure (e.g., a compound of Formula (I), (I-A), (I-A-I), (I-B), or Table 1) or a pharmaceutically acceptable salt thereof and a pharmaceutically acceptable carrier.

In some embodiments, a pharmaceutical composition, as described herein, comprises one or more compounds selected from Table 1, or a pharmaceutically acceptable salt thereof or stereoisomer thereof.

In some embodiments, a pharmaceutical composition, as described herein, comprises one or more compounds selected from Table 1, or a pharmaceutically acceptable salt thereof.

In some embodiments of the present disclosure, a pharmaceutical composition comprising one or more compounds of the present disclosure (e.g., a compound of Formula (I), (I-A), (I-A-I), (I-B), or Table 1) or a pharmaceutically acceptable salt thereof, and a pharmaceutically acceptable excipient or adjuvant is provided. The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

Generally, the compounds of the present disclosure are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

### Methods of Treatment

The compounds of the present disclosure find use in any number of methods. For example, in some embodiments the compounds are useful in methods for modulating an orexin receptor, e.g., orexin type 2 receptor. Accordingly, in some embodiments, the present disclosure provides the use of any one of the foregoing compounds of Formula (I), (I-A), (I-A-I), (I-B), or Table 1 or a pharmaceutically acceptable salt thereof, for modulating orexin receptor (e.g., orexin type 2 receptor) activity. For example, in some embodiments, modulating orexin receptor (e.g., orexin type 2 receptor) activity is in a mammalian cell. Modulating orexin receptor (e.g., orexin type 2 receptor) activity can be in a subject in need thereof (e.g., a mammalian subject, such as a human) and for treatment of any of the described conditions or diseases.

In some embodiments, the modulating orexin receptor (e.g., orexin type 2 receptor) activity is binding. In some embodiments, the modulating orexin receptor (e.g., orexin type 2 receptor) activity is agonizing or stimulating the orexin receptor.

In some embodiments, the present disclosure provides methods of treating a disease or disorder that is treatable by administration of an Orexin agonist, the method comprising administering a therapeutically effective amount of one or more compounds of the present disclosure (e.g., compounds of Formula (I), (I-A), (I-A-I), (I-B), or Table 1) or a pharmaceutically acceptable salt thereof.

In some embodiments, the compounds of the present disclosure are used for treating, preventing, ameliorating, controlling or reducing the risk of a variety of disorders associated with orexin receptors, including one or more of the following conditions or diseases: narcolepsy, narcolepsy syndrome accompanied by narcolepsy-like symptoms, cataplexy in narcolepsy, excessive daytime sleepiness (EDS) in narcolepsy, hypersomnia, idiopathic hypersomnia, repeatability hypersomnia, intrinsic hypersomnia, hypersomnia accompanied by daytime hypersomnia, interrupted sleep, sleep apnea, hypersomnia associated with sleep apnea, nocturnal myoclonus, disturbances of consciousness, such as coma, REM sleep interruptions, jet-lag, excessive daytime sleepiness, shift workers' sleep disturbances, dyssomnias, sleep disorders, sleep disturbances, hypersomnia associated with depression, emotional/mood disorders, drug use, Alzheimer's disease or cognitive impairment, Parkinson's disease, Guillain-Barre syndrome, Kleine Levin syndrome, and sleep disorders which accompany aging, muscular dystrophies, immune-mediated diseases; Alzheimer's sundowning; conditions associated with circadian rhythmicity as well as mental and physical disorders associated with travel across time zones and with rotating shift-work schedules; fibromyalgia; cardiac failure; diseases related to bone loss; sepsis; syndromes which are manifested by non-restorative sleep and muscle pain or sleep apnea which is associated with respiratory disturbances during sleep; conditions which result from a diminished quality of sleep; and other diseases related to general orexin system dysfunction. In some embodiments, compounds of the present disclosure are useful for treating, preventing, ameliorating, controlling or reducing the risk of a variety of narcolepsy, idiopathic hypersomnia, hypersomnia, sleep apnea syndrome, narcolepsy syndrome accompanied by narcolepsy-like symptoms, hypersomnia syndrome accompanied by daytime hypersomnia (e.g., Parkinson's disease, Guillain-Barre syndrome and Kleine Levin syndrome), Alzheimer, obesity, insulin resistance syndrome, cardiac failure, diseases related to bone loss, sepsis, disturbance of consciousness such as coma and the like, side effects and complications due to anesthesia, and the like, or anesthetic antagonist.

In some embodiments, a compound of the present disclosure (e.g., a compound of Formula (I), (I-A), (I-A-I), (I-B), or Table 1) or a pharmaceutically acceptable salt thereof, are used to treat diseases or disorders or symptoms associated with excessive sleepiness in a subject in need thereof. In some embodiments, the excessive sleepiness is caused by any one of the following: insufficient quality or quantity of night time sleep; misalignments of the body's circadian pacemaker with the environment (e.g., caused by requirement to remain awake at night for employment such as shift work or personal obligations such as caretaker for sick, young or old family members), such as jet lag, shift work and other circadian rhythm sleep disorders; another underlying sleep disorder, such as narcolepsy (e.g., narcolepsy type 1, narcolepsy type 2, probable narcolepsy), sleep apnea (e.g., obstructive sleep apnea, obstructive sleep apnea with use of continuous positive airway pressure), idiopathic hypersomnia, idiopathic excessive sleepiness, and restless legs syndrome; disorders, such as clinical depression or atypical depression; tumors; head trauma; anemia; kidney failure; hypothyroidism; injury to the central nervous system; drug abuse; genetic vitamin deficiency, such as biotin deficiency; and particular classes of prescription and over the counter medication.

In some embodiments, a compound of the present disclosure (e.g., a compound of Formula (I), (I-A), (I-A-I), (I-B), or Table 1) or a pharmaceutically acceptable salt thereof), are used treat any one of the following: shift work disorder; shift work sleep disorder; and jet lag syndrome. In some embodiments, the methods and uses herein are used to treat any one of the following: narcolepsy type 1, narcolepsy type 2, probable narcolepsy, idiopathic hypersomnia, idiopathic excessive sleepiness, hypersomnia, hypersomnolence, sleep apnea syndrome (e.g., obstructive sleep apnea, obstructive sleep apnea with use of continuous positive airway pressure); or disturbance of consciousness such as coma and the like; and narcolepsy syndrome accompanied by narcolepsy-like symptoms; hypersomnolence or hypersomnia syndrome accompanied by daytime hypersomnia (e.g., Parkinson's disease, Guillain-Barre syndrome and Kleine Levin syndrome); excessive daytime sleepiness in Parkinson's disease, Prader-Willi Syndrome, depressions (depression, atypical depression, major depressive disorder, treatment resistant depression), ADHD, sleep apnea syndrome (e.g., obstructive sleep apnea, obstructive sleep apnea with use of continuous positive airway pressure) and other disorders of vigilance; residual excessive daytime sleepiness in sleep apnea syndrome (e.g., obstructive sleep apnea, obstructive sleep apnea with use of continuous positive airway pressure); and the like. Narcolepsy (e.g., narcolepsy type 1, narcolepsy type 2, probable narcolepsy) may be diagnosed by diagnostic criteria generally used in the field, e.g., The third edition of the International Classification of Sleep Disorders (ICSD-3) and the fifth edition of the Diagnostic and Statistical Manual of Mental Disorders (DSM-5). In some embodiments, the excessive sleepiness is excessive daytime sleepiness or excessive sleepiness during working hours, or excessive sleepiness or reduced quantity of sleep which is caused by requirement to remain awake at night for employment (e.g., shift work) or personal obligations (e.g., caretaker for sick, young or old family members). In some embodiments, the subject suffers from the diseases or disorders or symptoms associated with excessive sleepiness. In some embodiments, the subject is sleep-deprived subject, subject with excessive sleepiness, subject with disruptive regular sleep cycle, or subject with a need to decrease sleepiness. In some embodiments, the present disclosure provides methods for decreasing or treating excessive sleepiness. In some embodiments, the excessive sleepiness is caused by narcolepsy type 1, narcolepsy type 2 or idiopathic hypersomnia. In some embodiments, the excessive sleepiness is caused by obstructive sleep apnea despite the use of continuous positive airway pressure (CPAP). In some embodiments, methods for increasing wakefulness in a subject in need thereof is provided. In some embodiments, the orexin level in the subject is not compromised or partially compromised.

In some embodiments of the present disclosure, a method for the treatment of a sleep disorder (e.g., as disclosed herein) in a subject in need thereof is provided, comprising administering a compound of the present disclosure (e.g., a compound of Formula (I), (I-A), (I-A-I), (I-B), or Table 1) or a pharmaceutically acceptable salt thereof, to a subject in need thereof. In some embodiments, a compound of the present disclosure (e.g., a compound of Formula (I), (I-A), (I-A-I), (I-B), or Table 1) or a pharmaceutically acceptable salt thereof, is used to treat a subject with a sleep disorder, to treat a sleep disorder, or to treat the symptoms of a sleep disorder.

In some embodiments of the present disclosure, a method for the treatment of narcolepsy in a subject in need thereof is provided, comprising administering a compound of the present disclosure (e.g., a compound of Formula (I), (I-A), (I-A-I), (I-B), or Table 1) or a pharmaceutically acceptable salt thereof, to a subject in need thereof. In some embodiments, a compound of the present disclosure (e.g., a compound of Formula (I), (I-A), (I-A-I), (I-B), or Table 1) or a pharmaceutically acceptable salt thereof, is used to treat a subject with narcolepsy, to treat narcolepsy, or to treat the symptoms of narcolepsy.

In some embodiments of the present disclosure, a method for the treatment of idiopathic hypersomnia (IH) in a subject in need thereof is provided, comprising administering a compound of the present disclosure (e.g., a compound of Formula (I), (I-A), (I-A-I), (I-B), or Table 1) or a pharmaceutically acceptable salt thereof, to a subject in need thereof. In some embodiments, a compound of the present disclosure (e.g., a compound of Formula (I), (I-A), (I-A-I), (I-B), or Table 1) or a pharmaceutically acceptable salt thereof, is used to treat a subject with IH, to treat IH, or to treat the symptoms of IH.

### EXAMPLES

The disclosure now being generally described, it will be more readily understood by reference to the following examples which are included merely for purposes of illustration of certain aspects and embodiments of the present disclosure and are not intended to limit the present disclosure.

The compounds of the present disclosure can be synthesized using the methods as hereinafter described below, together with synthetic methods known in the art of synthetic organic chemistry or variations thereon as appreciated by those skilled in the art.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

### IP-1 Accumulation Assay

The accumulation of Inositol-1 Monophosphate (IP-1) was measured using IP-One HTRF® Terbium cryptate based assay (Cisbio) in human recombinant OX1 (hOX1) and at OX2 (hOX2) receptors expressed in CHO cells (DiscoverX) according to the manufacturer's instructions for cells tested in suspension.

hOX1-CHO and hOX2-CHO cells were seeded into white 384-well plates at a density of 20,000 cells/well in Hank's Balanced Salt Solution (HBSS) containing 20 mM HEPES pH 7.4, 50 mM, LiCl and 0.1% and Bovine Serum Albumin (BSA).

Compounds of the present disclosure were tested in a 11 points concentration response curve (CRC) serially diluted in neat DMSO at 200 fold concentrations and added by Echo acoustic liquid handling (Labcyte) to the cells (0.5% DMSO final in the assay). After 60 min of incubation at 37° C. detection reagents, IP1-d2 tracer and anti-IP1-cryptate were diluted in lysis buffer according to the manufacturer's descriptions and added to the cells.

Following 60 min incubation at room temperature, time-resolved fluorescence (HTRF) was measured at 615 nm and 665 nm by Envision Multilabel reader (Perkin Elmer) and the HTRF ratio (A665/A615x104) was calculated.

The IP-1 accumulation response was expressed as percentage of the maximal OX-A response.

Curve fitting and EC50 estimations were carried out using a four-parameter logistic model using XLfit Software. Mean data of EC50 are calculated from at least two independent experiments performed in duplicates.

In the Tables, below Category A corresponds to compounds displaying an IC50<100 nM, Category B between 100 nM and 1,000 nM, Category C between 1,000 nM and 10,000 nM and Category D above 10,000 nM.

### Example 1: N-[2-(2-oxo-1,2-dihydropyridin-1-yl)-3-{[(CIS)-4-phenylcyclohexyl]oxy}propyl]methanesulfonamide (1)

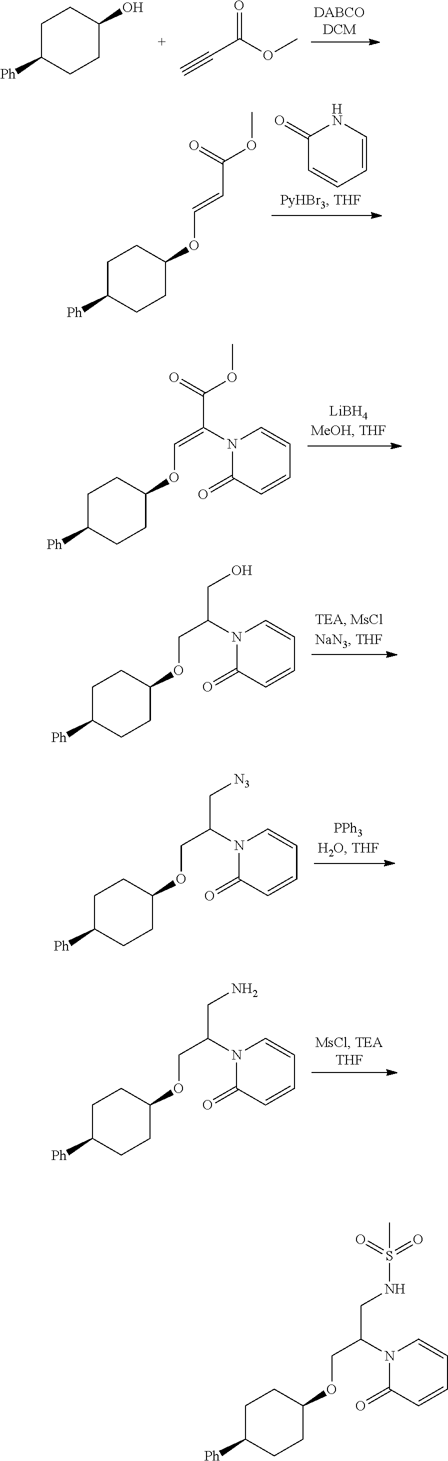

### Methyl (2E)-3-{[(CIS)-4-phenylcyclohexyl]oxy}prop-2-enoate (INTERMEDIATE 1)

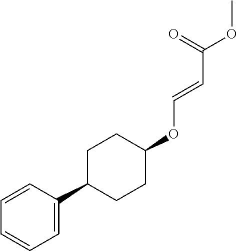 [SMILES: COC(=O)/C=C/O[C@H]1CC[C@@H](c2ccccc2)CC1]

Cis-4-phenylcyclohexan-1-ol (2.04 g, 11.57 mmol) and DABCO (0.13 g, 1.16 mmol) were stirred in DCM (45 mL) at 0° C. while 2-propynoic acid methyl ester (1.34 mL, 15.05 mmol) was added dropwise over 5 minutes. The solution was stirred at room temperature for 30 minutes. The solvent was removed in vacuo and the residue was purified by column chromatography using a C18 cartridge (H₂O+0.1% formic acid/MeCN+0.1% formic acid, 80:20 to 0:100) to afford the title compound (2.31 g, 8.87 mmol, 77% yield) as an off-white solid. [M+H]⁺ m/z 261.2

### Methyl (2Z)-2-(2-oxo-1,2-dihydropyridin-1-yl)-3-{[(CIS)-4-phenylcyclohexyl]oxy}prop-2-enoate (INTERMEDIATE 2)

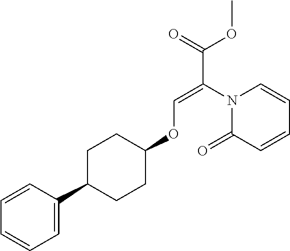 [SMILES: COC(=O)/C(=C/O[C@H]1CC[C@@H](c2ccccc2)CC1)n1ccccc1=O]

To a solution of methyl Intermediate 1 (3.05 g, 11.72 mmol) in anhydrous THF (55 ml) at 0° C., a solution of pyridinium tribromide (3.93 g, 12.3 mmol) in THF (10 ml) was added over a period of 10 minutes. The reaction mixture was allowed to warm at room temperature and stirred for 30 minutes. 1H-pyridin-2-one (2.79 g, 29.29 mmol) was added and the mixture was stirred at room temperature for 16 h. The solvent was removed in vacuo and the product was purified by column chromatography using a silica cartridge (0-100% EtOAc in cHex) to afford the title compound (1.62 g, 4.58 mmol, 39% yield) as a colourless glassy solid. [M+H]⁺ m/z 354.2

### 1-(1-Hydroxy-3-{[(CIS)-4-phenylcyclohexyl]oxy}propan-2-yl)-1,2-dihydropyridin-2-one (INTERMEDIATE 3)

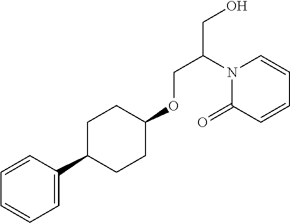 [SMILES: O=c1ccccn1C(CO)CO[C@H]1CC[C@@H](c2ccccc2)CC1]

To a solution of Intermediate 2 (1.52 g, 4.3 mmol) in THF (50 mL) and MeOH (5 mL) was added NaBH₄ (814 mg, 21.5 mmol). The mixture was heated at 80° C. and after 1 h acetone was added at room temperature. The solvent was removed in vacuo, then H₂O and 1M aqueous HCl were added. The mixture was extracted with EtOAc, then the combined organic layer was evaporated in vacuo to afford the title compound (1.56 g, quant. yield) as a white foam. [M+H]⁺ m/z 328.2.

### 1-(1-Azido-3-{[(CIS)-4-phenylcyclohexyl]oxy}propan-2-yl)-1,2-dihydropyridin-2-one (INTERMEDIATE 4)

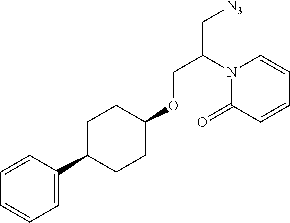 [SMILES: [N-]=[N+]=NCC(CO[C@H]1CC[C@@H](c2ccccc2)CC1)n1ccccc1=O]

To a solution of Intermediate 3 (823 mg, 2.41 mmol) in THF (15 mL) at 0° C., TEA (0.5 mL, 3.62 mmol) and methanesulfonyl chloride (0.21 mL, 2.65 mmol) were added. After 30 minutes NaN₃ (29 mg, 0.440 mmol) was added at 0° C. The mixture was allowed to warm to room temperature and stirred overnight. After 18 h the mixture was diluted with H₂O and extracted twice with EtOAc. The combined organic layer was evaporated in vacuo to afford the title compound (877 mg, quant. yield) as a pale pink oil. [M+H]⁺ m/z 353.2

### 1-(1-Amino-3-{[(CIS)-4-phenylcyclohexyl]oxy}propan-2-yl)-1,2-dihydropyridin-2-one (INTERMEDIATE 5)

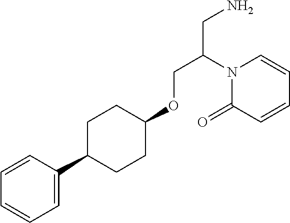 [SMILES: NCC(CO[C@H]1CC[C@@H](c2ccccc2)CC1)n1ccccc1=O]

To a stirred solution of Intermediate 4 (0.88 g, 2.49 mmol) in THF (9 mL), PPh₃ (1.31 g, 4.98 mmol) and H₂O (4 mL) were added. The mixture was stirred at room temperature for 4 hours, then it was diluted with water and extracted twice with EtOAc. The combined organic layers were evaporated in vacuo. The product was purified using a SCX cartridge (loaded with MeOH, washed with MeOH, and recovered using 2 M NH₃ in MeOH) to afford a pink oil that was further purified by column chromatography using a silica cartridge (0-5% methanol in DCM) to afford the title compound (0.24 g, 0.74 mmol, 30% yield) as a pale-yellow oil. [M+H]⁺ m/z 327.5

### N-[2-(2-oxo-1,2-dihydropyridin-1-yl)-3-{[(CIS)-4-phenylcyclohexyl]oxy}propyl]methanesulfonamide (1)

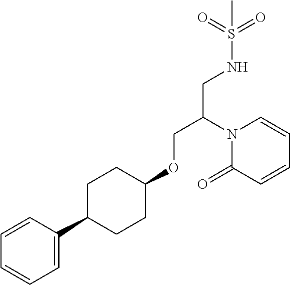 [SMILES: CS(=O)(=O)NCC(CO[C@H]1CC[C@@H](c2ccccc2)CC1)n1ccccc1=O]

To a stirred solution of Intermediate 5 (15 mg, 0.050 mmol) in THF (0.6 mL), TEA (12.81 μL, 0.090 mmol) was added at room temperature followed by methanesulfonyl chloride (4 μL, 0.060 mmol). The mixture was stirred at room temperature and after 30 minutes the solvent was removed in vacuo. The product was purified by column chromatography using a C18 cartridge (H₂O+0.1% formic acid/MeCN+0.1% formic acid, 95:5 to 40:60) to afford the title compound (12.8 mg, 0.032 mmol, 69% yield) as a white solid.

### Examples 1a-1e

Examples 1a-1e in Table 2 below were prepared following the procedure described in Example 1 using the appropriate heterocyclic reagents. The starting materials are either prepared as described in the intermediates section, commercially available, or may be prepared from commercially available reagents using conventional reactions well known in the art. Enantiomers were separated by chiral purification using appropriate columns Chiralpak columns (AD-H, IC, OJ-H), and eluting with a mixture of n-Hexane/EtOH, n-Hexane/(EtOH+0.1% iPrNH2), n-Hexane/(EtOH/MeOH 1:1+0.1% iPrNH2), with ratios between 35:65 and 70:30. Isomer 1 was assigned to the first eluting compound and Isomer 2 to the second eluting compound during chiral separation using one of the conditions reported above. The products are reported in the table below:

TABLE 2
Characterization and EC50 hOX2 data-Compounds 1-1e:

| | | | Obs. | | EC50 |
|---|---|---|---|---|---|
| Ex. | Structure | Name | Mass | ¹H NMR | hOX2 |
| 1 | 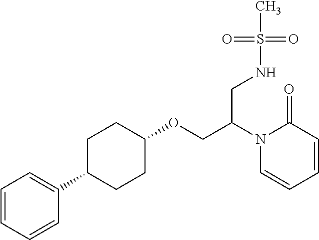 [SMILES: CS(=O)(=O)NCC(CO[C@H]1CC[C@@H](c2ccccc2)CC1)n1ccccc1=O] | N-[2-(2-oxo- 1,2- dihydropyridin- 1-yl)-3- {[(CIS)-4- phenylcyclo- hexyl]oxy} propyl] methane- sulfonamide | 405.2 [M + H]⁺ | ¹H NMR (500 MHz, CDCl₃) δ 7.68 (dd, J = 6.9, 1.8 Hz, 1H), 7.37 (ddd, J = 9.0, 6.8, 1.9 Hz, 1H), 7.33-7.28 (m, 2H), 7.22-7.16 (m, 3H), 6.60 (d, J = 9.2 Hz, 1H), 6.26 (td, J = 6.8, 1.4 Hz, 1H), 5.21-5.01 (m, 2H), 3.90 (dd, J = 10.4, 5.4 Hz, 1H), 3.80 (dd, J = 10.3, 3.7 Hz, 1H), 3.78-3.73 (m, 1H), 3.71-3.63 (m, 2H), 2.90 (s, 3H), 2.63-2.45 (m, 1H), 2.09-1.93 (m, 2H), 1.75-1.62 (m, 4H), 1.61- 1.49 (m, 2H). | C |
| 1a | 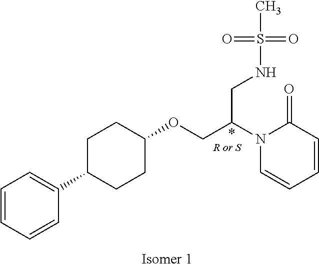 [SMILES: CS(=O)(=O)NCC(CO[C@H]1CC[C@@H](c2ccccc2)CC1)n1ccccc1=O] | (R or S) N-[2- (2-oxo-1,2- dihydropyridin- 1-yl)-3- {[(CIS)-4- phenylcyclo- hexyl] oxy}propyl] methane- sulfonamide- Isomer 1 | 405.2 [M + H]⁺ | ¹H NMR (400 MHz, CDCl₃) δ 7.70- 7.64 (m, 1H), 7.36 (ddd, J = 8.9, 6.6, 2.1 Hz, 1H), 7.29 (dd, J = 8.1, 6.9 Hz, 2H), 7.22-7.15 (m, 3H), 6.59 (dt, J = 9.0, 1.2 Hz, 1H), 6.25 (td, J = 6.8, 1.4 Hz, 1H), 5.18 (t, J = 6.2 Hz, 1H), 5.11 (dt, J = 8.8, 4.6 Hz, 1H), 3.89 (dd, J = 10.3, 5.4 Hz, 1H), 3.81-3.71 (m, 2H), 3.71-3.61 (m, 2H), 2.89 (s, 3H), 2.52 (p, J = 7.8 Hz, 1H), 2.05- 1.95 (m, 2H), 1.71-1.61 (m, 4H), 1.56 (s, 2H). | C |
| 1b | 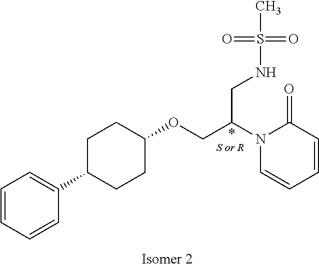 [SMILES: CS(=O)(=O)NCC(CO[C@H]1CC[C@@H](c2ccccc2)CC1)n1ccccc1=O] | (S or R) N-[2- (2-oxo-1,2- dihydropyridin- 1-yl)-3- {[(CIS)-4- phenylcyclo- hexyl] oxy}propyl] methane- sulfonamide- Isomer 2 | 405.2 [M + H]⁺ | ¹H NMR (400 MHz, CDCl₃) δ 7.67 (dd, J = 6.8, 2.1 Hz, 1H), 7.36 (ddd, J = 9.0, 6.6, 2.0 Hz, 1H), 7.29 (t, J = 7.5 Hz, 2H), 7.22-7.15 (m, 3H), 6.59 (d, J = 9.1 Hz, 1H), 6.28-6.22 (m, 1H), 5.14-5.01 (m, 2H), 3.90 (dd, J = 10.4, 5.4 Hz, 1H), 3.84-3.71 (m, 2H), 3.71-3.61 (m, 2H), 2.89 (s, 3H), 2.53 (t, J = 7.7 Hz, 1H), 2.00 (d, J = 13.8 Hz, 2H), 1.71-1.61 (m, 4H), 1.54 (s, 2H). | D |
| 1c | 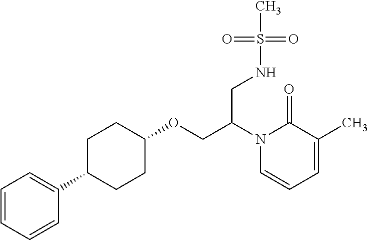 [SMILES: Cc1cccn(C(CNS(C)(=O)=O)CO[C@H]2CC[C@@H](c3ccccc3)CC2)c1=O] | N-[2-(3- methyl-2-oxo- 1,2- dihydropyridin- 1-yl)-3- {[(CIS)-4- phenylcyclo- hexyl] oxy}propyl] methane- sulfonamide | 419.3 [M + H]⁺ | ¹H NMR (400 MHz, CDCl₃) δ 7.53 (d, J = 7.2 Hz, 1H), 7.28 (d, J = 13.4 Hz, 3H), 7.20-7.14 (m, 3H), 6.17 (t, J = 6.8 Hz, 1H), 5.11 (d, J = 4.9 Hz, 2H), 3.89 (dd, J = 10.3, 5.5 Hz, 1H), 3.84-3.61 (m, 4H), 2.88 (s, 3H), 2.51 (q, J = 9.2, 7.7 Hz, 1H), 2.15 (s, 3H), 2.03-1.96 (m, 2H), 1.74-1.60 (m, 4H). | B |
| 1d | 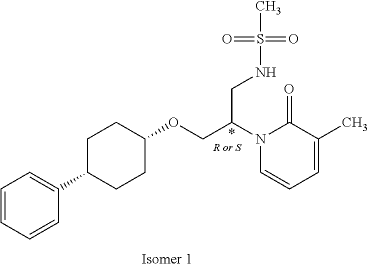 [SMILES: Cc1cccn(C(CNS(C)(=O)=O)CO[C@H]2CC[C@@H](c3ccccc3)CC2)c1=O] | (R or S) N-[2- (3-methyl-2- oxo-1,2- dihydropyridin- 1-yl)-3- {[(CIS)-4- phenylcyclo- hexyl] oxy}propyl] methane- sulfonamide- Isomer 1 | 419.2 [M + H]⁺ | ¹H NMR (400 MHz, CDCl₃) δ 7.56- 7.51 (m, 1H), 7.29 (dd, J = 8.1, 6.9 Hz, 2H), 7.24 (ddd, J = 6.7, 2.0, 1.1 Hz, 1H), 7.21-7.14 (m, 3H), 6.17 (t, J = 6.8 Hz, 1H), 5.11 (dt, J = 9.2, 4.7 Hz, 1H), 3.89 (dd, J = 10.3, 5.4 Hz, 1H), 3.79 (dd, J = 10.3, 4.0 Hz, 1H), 3.76-3.60 (m, 3H), 2.88 (s, 3H), 2.58-2.46 (m, 1H), 2.15 (s, 3H), 1.99 (ddd, J = 11.9, 6.2, 3.0 Hz, 2H), 1.73-1.44 (m, 6H). NH not visible. | D |
| 1e | 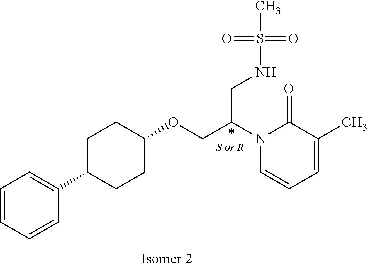 [SMILES: Cc1cccn(C(CNS(C)(=O)=O)CO[C@H]2CC[C@@H](c3ccccc3)CC2)c1=O] | (S or R) N- [(2R)-2-(3- methyl-2-oxo- 1,2- dihydropyridin- 1-yl)-3- {[(CIS)-4- phenylcyclo- hexyl] oxy}propyl] methane- sulfonamide- Isomer 2 | 419.2 [M + H]⁺ | ¹H NMR (400 MHz, CDCl₃) δ 7.56- 7.50 (m, 1H), 7.29 (t, J = 7.5 Hz, 2H), 7.25-7.21 (m, 1H), 7.22-7.14 (m, 3H), 6.17 (t, J = 6.8 Hz, 1H), 5.17- 4.98 (m, 2H), 3.89 (dd, J = 10.3, 5.4 Hz, 1H), 3.79 (dd, J = 10.3, 4.0 Hz, 1H), 3.76-3.60 (m, 3H), 2.88 (s, 3H), 2.57-2.44 (m, 1H), 2.15 (s, 3H), 2.06-1.94 (m, 2H), 1.55 (s, 6H). | B |

### Example 1f: 1-{1-[(Dimethylsulfamoyl)amino]-3-{[(CIS)-4-phenylcyclohexyl]oxy}propan-2-yl}-1,2-dihydropyridin-2-one (1f)

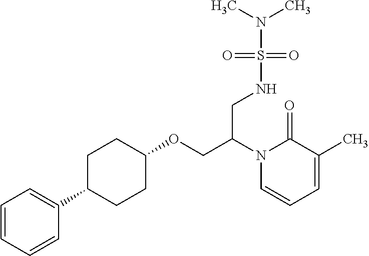 [SMILES: Cc1cccn(C(CNS(=O)(=O)N(C)C)CO[C@H]2CC[C@@H](c3ccccc3)CC2)c1=O]

To a stirred solution of Intermediate 5 (30 mg, 0.090 mmol) in THF (3.4 mL), TEA (26 μL, 0.18 mmol) was added at room temperature followed by N,N-dimethylsulfamoyl chloride (12 μL, 0.11 mmol). The mixture was stirred at 50° C. for 24 hours. Additional N,N-dimethylsulfamoyl chloride (12 μL, 0.11 mmol) was added and the mixture was stirred at 50° C. for 20 h. The solvent was removed in vacuo. The product was purified by HPLC preparative chromatography to afford the title compound (4.5 mg, 0.010 mmol, 11% yield) as a white solid.

TABLE 3
Characterization and EC50 hOX2 data-Compound 1f

| | | | Obs. | | EC50 |
|---|---|---|---|---|---|
| Ex. | Structure | Name | Mass | ¹H NMR | hOX2 |
| 1f | 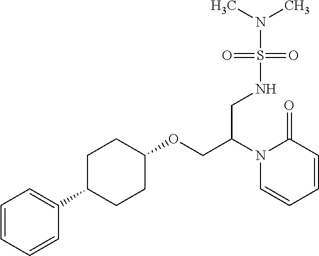 [SMILES: CN(C)S(=O)(=O)NCC(CO[C@H]1CC[C@@H](c2ccccc2)CC1)n1ccccc1=O] | 1-{1-[(dimethyl- sulfamoyl) amino]-3- {[(CIS)-4- phenylcyclo- hexyl]oxy} propan-2-yl}- 1,2-dihydro- pyridin-2-one | 434.3 [M + H]⁺ | ¹H NMR (400 MHz, CDCl₃) δ 7.66 (dd, J = 7.0, 2.0 Hz, 1H), 7.35 (ddd, J = 8.9, 6.5, 2.0 Hz, 1H), 7.29 (t, J = 8.0 Hz, 2H), 7.12-7.20 (m, 3H), 6.58 (dt, J = 9.0, 1.2 Hz, 1H), 6.23 (td, J = 6.8, 1.4 Hz, 1H), 5.09 (dt, J = 9.1, 4.6 Hz, 1H), 4.91 (t, J = 6.1 Hz, 1H), 3.89 (dd, J = 10.3, 5.3 Hz, 1H), 3.78 (dd, J = 10.3, 3.8 Hz, 1H), 3.51-3.70 (m, 3H), 2.74 (s, 6H), 2.53 (dd, J = 14.7, 7.1 Hz, 1H), 1.93-2.04 (m, 2H), 1.64 (dt, J = 7.8, 3.7 Hz, 6H). | C |

### Example 2: N-[2-(3-ethyl-2-oxo-1,2-dihydropyridin-1-yl)-3-{[(CIS)-4-phenylcyclohexyl]oxy}propyl]methanesulfonamide

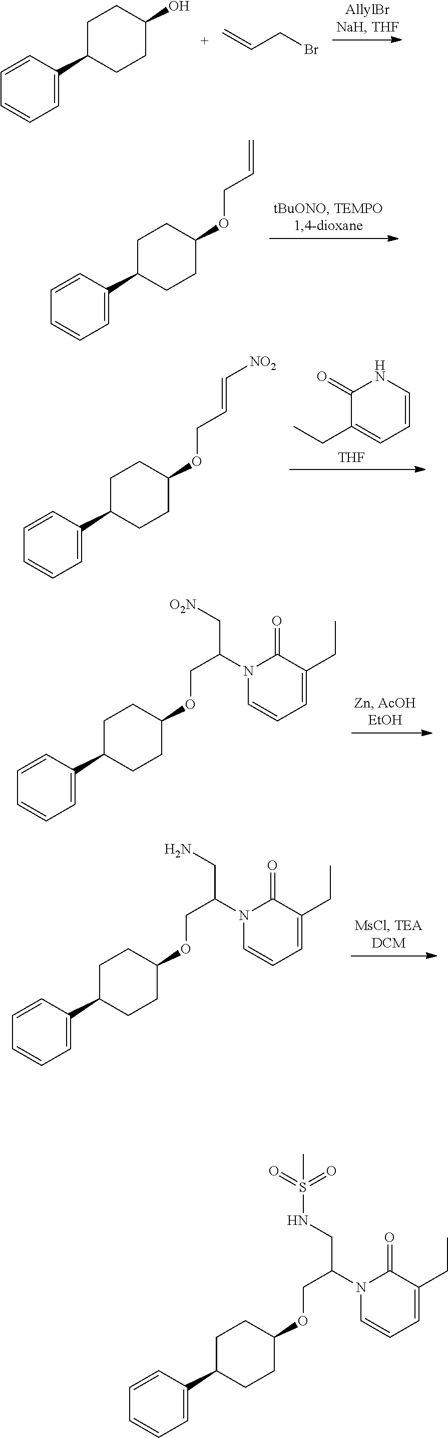

### CIS-4-(prop-2-en-1-yloxy)cyclohexyl]benzene (INTERMEDIATE 6)

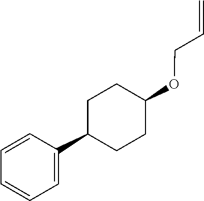 [SMILES: C=CCO[C@H]1CC[C@@H](c2ccccc2)CC1]

To a mixture of cis-4-phenylcyclohexan-1-ol (3.0 g, 17.02 mmol) in anhydrous THF (50 mL) was added NaH (0.89 g, 22.13 mmol) (60% w/w dispersion in mineral oil) at room temperature. The mixture was stirred under N₂ atmosphere at room temperature for 45 minutes, then allyl bromide (2.21 mL, 25.54 mmol) was added. The mixture was stirred at 60° C. for 5 h, then it was poured into ice and extracted with EtOAc (3×150 mL). The organic layer was dried over Na₂SO₄ and evaporated in vacuo. The product was purified by column chromatography using a silica cartridge (cHex/EtOAc from 100:0 to 70:30) to afford the title compound (3.35 g, 15.49 mmol, 91% yield) as a pale yellow oil. [M-allyl-H₂O]⁺ m/z 159.0.

### [CIS-4-{[(2E)-3-nitroprop-2-en-1-yl]oxy}cyclohexyl]benzene (INTERMEDIATE 7)

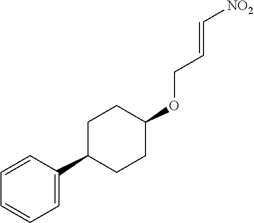 [SMILES: O=[N+]([O-])/C=C/CO[C@H]1CC[C@@H](c2ccccc2)CC1]

To a suspension of Intermediate 6 (688 mg, 3.18 mmol) in anhydrous 1,4-dioxane (25 mL) stirring at room temperature and under air, TEMPO (200 mg, 1.27 mmol) and tBuONO (0.76 mL, 6.36 mmol) were added. The mixture was stirred at 50° C. for 2.5 h, then it was diluted with EtOAc (100 mL) and washed with a solution of saturated aqueous NH₄Cl (2×40 mL). The organic layer was dried with a phase separator and concentrated in vacuo. The product was purified by column chromatography using a silica cartridge (0-15% EtOAc in cHex) and then using a C₁₈ cartridge (H₂O+0.1% formic acid/MeCN+0.1% formic acid from 100:0 to 0:100) to afford the title compound (300 mg, 1.15 mmol, 36% yield) as a pale-yellow oil. ¹H NMR (400 MHz, CDCl₃) δ 7.39-7.28 (m, 4H), 7.25-7.17 (m, 3H), 4.27 (dd, J=3.2, 2.0 Hz, 2H), 3.72 (t, J=3.1 Hz, 1H), 2.56 (tt, J=11.9, 3.7 Hz, 1H), 2.05 (dt, J=14.7, 2.8 Hz, 2H), 1.89-1.76 (m, 2H), 1.72-1.55 (m, 4H).

### 3-Ethyl-1-(1-nitro-3-{[(CIS)-4-phenylcyclohexyl]oxy}propan-2-yl)-1,2-dihydropyridin-2-one (INTERMEDIATE 8)

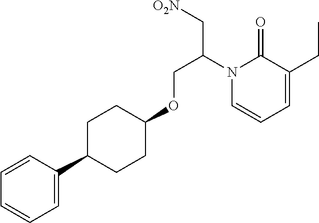 [SMILES: CCc1cccn(C(CO[C@H]2CC[C@@H](c3ccccc3)CC2)C[N+](=O)[O-])c1=O]

3-Ethyl-1H-pyridin-2-one (188 mg, 1.53 mmol) and Intermediate 7 (200 mg, 0.77 mmol) were dissolved in THF (14 mL). The solution was stirred at 80° C. for 39 h. The mixture was diluted with H₂O and extracted with EtOAc. The organic layers were evaporated in vacuo to afford the title compound (357 mg, quant. yield) as a yellow oil. [M+H]⁺ m/z 385.2.

### 1-(1-Amino-3-{[(CIS)-4-phenylcyclohexyl]oxy}propan-2-yl)-3-ethyl-1,2-dihydropyridin-2-one (INTERMEDIATE 9)

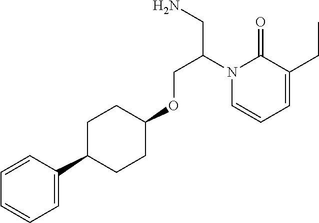 [SMILES: CCc1cccn(C(CN)CO[C@H]2CC[C@@H](c3ccccc3)CC2)c1=O]

To a solution of Intermediate 8 (350 mg, 0.91 mmol) in EtOH (8 mL), AcOH (1.3 mL), and Zn powder (595 mg, 9.1 mmol) were added and the mixture was stirred for 45 minutes at room temperature. The mixture was filtered through Celite and washed with MeOH. The filtrate was concentrated in vacuo and NaOH (2M in H₂O) was added. The mixture was extracted with EtOAc and the organic layer was evaporated in vacuo to afford a crude that was further purified by SCX (charged in MeOH, washed in MeOH and recovered with MeOH/NH₄ 4 M). The solvent was removed in vacuo to afford the title compound (202 mg, 0.57 mmol, 63% yield) as a yellow oil. [M+H]⁺ m/z 356.6.

### N-[2-(3-ethyl-2-oxo-1,2-dihydropyridin-1-yl)-3-{[(CIS)-4-phenylcyclohexyl]oxy}propyl]methanesulfonamide (2)

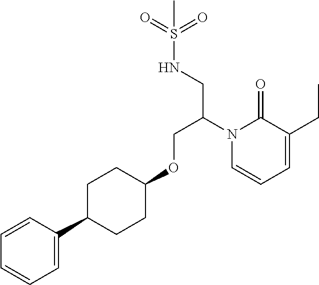 [SMILES: CCc1cccn(C(CNS(C)(=O)=O)CO[C@H]2CC[C@@H](c3ccccc3)CC2)c1=O]

Example 2 was prepared following the procedure described for Example 1, starting from Intermediate 9 (50 mg, 0.14 mmol) to afford the title compound (34 mg, 0.078 mmol, 55% yield) as a colourless glassy solid.

Below examples were prepared following the same 3-steps procedure described for the synthesis of Example 2, using the corresponding commercially available starting materials which are either prepared as described in the intermediates section, commercially available, or may be prepared from commercially available reagents using conventional reactions well known in the art. Example 2aa was prepared following the 3-steps procedure used for the synthesis of Example 2, with the only variation of step 1, in which 3-methylpyrazole was reacted with Intermediate 7 in DCM at room temperature. Examples 2af, 2ag, 2ah and 2ai were prepared using F-phenylcyclohexanol following the same procedure described for the synthesis of Example 2.

Enantiomers were separated by chiral purification using Chiralpak columns (AD-H, IC, OJ-H), and eluting with a mixture of n-Hexane/EtOH, n-Hexane/(EtOH+0.1% iPrNH₂), n-Hexane/(EtOH/MeOH 1:1+0.1% iPrNH₂), with ratios between 35:65 and 70:30. Isomer 1 was assigned to the first eluting compound and Isomer 2 to the second eluting compound during chiral separation using one of the conditions reported above. The products are reported in the table below:

TABLE 4
Characterization and EC50 hOX2 data-Compounds 2-2aj

| | | | Obs. | | EC50 |
|---|---|---|---|---|---|
| Ex. | Structure | Name | Mass | ¹H NMR | hOX2 |
| 2 | 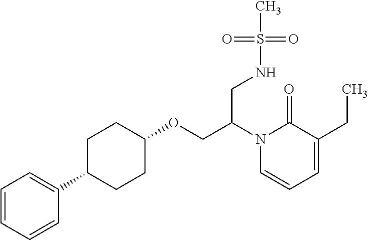 [SMILES: CCc1cccn(C(CNS(C)(=O)=O)CO[C@H]2CC[C@@H](c3ccccc3)CC2)c1=O] | N-[2-(3-ethyl- 2-oxo-1,2- dihydropyridin- 1-yl)-3- {[(CIS)-4- phenylcyclo- hexyl] oxy}propyl] methane- sulfonamide | 433.3 [M + H]⁺ | ¹H NMR (400 MHz, CDCl₃) δ 7.54 (dd, J = 7.0, 2.0 Hz, 1H), 7.32-7.25 (m, 2H), 7.25-7.14 (m, 4H), 6.21 (t, J = 6.9 Hz, 1H), 5.20-5.14 (m, 1H), 5.14-5.06 (m, 1H), 3.90 (dd, J = 10.3, 5.5 Hz, 1H), 3.83-3.61 (m, 4H), 2.87 (s, 3H), 2.61-2.46 (m, 3H), 2.05-1.95 (m, 2H), 1.71- 1.60 (m, 4H), 1.55-1.46 (m, 2H), 1.18 (t, J = 7.5 Hz, 3H). | C |
| 2a | 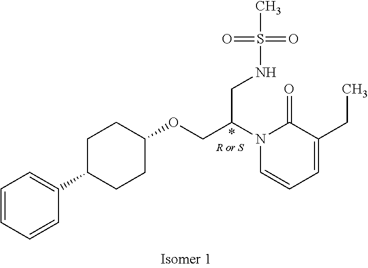 [SMILES: CCc1cccn(C(CNS(C)(=O)=O)CO[C@H]2CC[C@@H](c3ccccc3)CC2)c1=O] | (R or S) N-[2- (3-ethyl-2-oxo- 1,2- dihydropyridin- 1-yl)-3- {[(CIS)-4- phenylcyclo- hexyl] oxy}propyl] methane- sulfonamide- Isomer 1 | 433.2 [M + H]⁺ | ¹H NMR (400 MHz, CDCl₃) δ 7.56-7.50 (m, 1H), 7.32-7.27 (m, 2H), 7.23-7.14 (m, 4H), 6.21 (t, J = 6.8 Hz, 1H), 5.10 (s, 2H), 3.90 (dd, J = 10.3, 5.5 Hz, 1H), 3.83-3.60 (m, 4H), 2.87 (s, 3H), 2.60-2.47 (m, 3H), 1.98 (s, 2H), 1.54 (s, 6H), 1.18 (t, J = 7.4 Hz, 3H). | D |
| 2b | 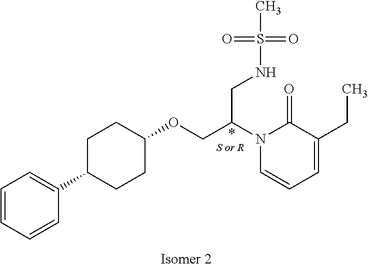 [SMILES: CCc1cccn(C(CNS(C)(=O)=O)CO[C@H]2CC[C@@H](c3ccccc3)CC2)c1=O] | (S or R) N-[2- (3-ethyl-2-oxo- 1,2- dihydropyridin- 1-yl)-3- {[(CIS)-4- phenylcyclo- hexyl] oxy}propyl] methane- sulfonamide- Isomer 2 | 433.2 [M + H]⁺ | ¹H NMR (400 MHz, CDCl₃) δ 7.54 (dd, J = 6.9, 1.9 Hz, 1H), 7.29 (t, J = 7.5 Hz, 2H), 7.24- 7.14 (m, 4H), 6.21 (t, J = 6.8 Hz, 1H), 5.11 (d, J = 6.3 Hz, 2H), 3.90 (dd, J = 10.3, 5.5 Hz, 1H), 3.84-3.59 (m, 4H), 2.87 (s, 3H), 2.65-2.40 (m, 3H), 2.12- 1.93 (m, 2H), 1.73-1.45 (m, 6H), 1.18 (t, J = 7.5 Hz, 3H). | B |
| 2c | 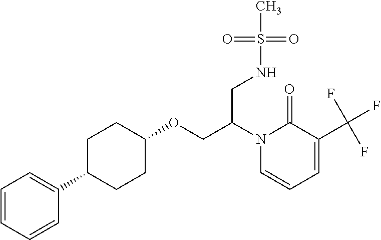 [SMILES: CS(=O)(=O)NCC(CO[C@H]1CC[C@@H](c2ccccc2)CC1)n1cccc(C(F)(F)F)c1=O] | N-{2-[2-oxo-3- (trifluoro- methyl)-1,2- dihydropyridin- 1-yl]-3- {[(CIS)-4- phenylcyclo- hexyl] oxy}propyl} methane- sulfonamide | 473.2 [M + H]⁺ | ¹H NMR (400 MHz, CDCl₃) δ 7.92 (dd, J = 7.0, 2.0 Hz, 1H), 7.78 (ddd, J = 7.0, 2.0, 0.90 Hz, 1H), 7.33-7.26 (m, 2H), 7.23- 7.14 (m, 3H), 6.32 (t, J = 7.0 Hz, 1H), 5.22 (t, J = 6.4 Hz, 1H), 5.15 (dt, J = 10.4, 4.4 Hz, 1H), 3.91 (dd, J = 10.6, 5.2 Hz, 1H), 3.82-3.73 (m, 2H), 3.69-3.60 (m, 2H), 2.92 (s, 3H), 2.52 (td, J = 10.2, 5.1 Hz, 1H), 2.05-1.91 (m, 2H), 1.74-1.41 (m, 6H). | C |
| 2d | 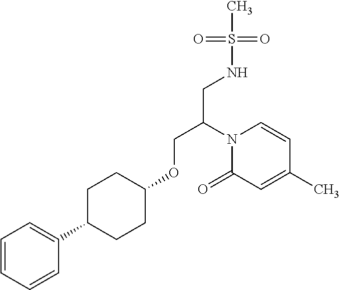 [SMILES: Cc1ccn(C(CNS(C)(=O)=O)CO[C@H]2CC[C@@H](c3ccccc3)CC2)c(=O)c1] | N-[2-(4- methyl-2-oxo- 1,2- dihydropyridin- 1-yl)-3- {[(CIS)-4- phenylcyclo- hexyl] oxy}propyl] methane- sulfonamide | 419.47 [M + H]⁺ | ¹H NMR (400 MHz, CDCl₃) δ 7.52 (d, J = 7.0 Hz, 1H), 7.29 (t, J = 7.5 Hz, 2H), 7.22-7.14 (m, 3H), 6.38 (s, 1H), 6.09 (dd, J = 7.1, 2.0 Hz, 1H), 5.14 (t, J = 6.2 Hz, 1H), 5.07-5.00 (m, 1H), 3.88 (dd, J = 10.3, 5.5 Hz, 1H), 3.74 (ddd, J = 13.1, 9.8, 5.3 Hz, 2H), 3.70-3.57 (m, 2H), 3.49 (s, 1H), 2.89 (s, 3H), 2.52 (p, J = 7.8 Hz, 1H), 2.18 (d, J = 1.0 Hz, 3H), 2.06-1.91 (m, 2H), 1.69- 1.60 (m, 5H). | C |
| 2e | 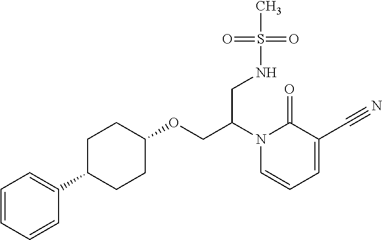 [SMILES: CS(=O)(=O)NCC(CO[C@H]1CC[C@@H](c2ccccc2)CC1)n1cccc(C#N)c1=O] | N-[2-(3-cyano- 2-oxo-1,2- dihydropyridin- 1-yl)-3- {[(CIS)-4- phenylcyclo- hexyl] oxy}propyl] methane- sulfonamide | 430.3 [M + H]⁺ | ¹H NMR (400 MHz, CDCl₃) δ 7.97 (dd, J = 6.9, 2.1 Hz, 1H), 7.83 (dd, J = 7.1, 2.0 Hz, 1H), 7.32-7.27 (m, 2H), 7.22-7.13 (m, 3H), 6.35 (t, J = 7.0 Hz, 1H), 5.33 (t, J = 6.5 Hz, 1H), 5.12 (h, J = 4.7 Hz, 1H), 3.90 (dd, J = 10.6, 5.3 Hz, 1H), 3.86-3.73 (m, 2H), 3.66 (dt, J = 13.9, 5.2 Hz, 2H), 3.17-3.06 (m, 1H), 2.96 (s, 3H), 2.53 (td, J = 10.9, 4.2 Hz, 1H), 2.05-1.91 (m, 2H), 1.72-1.60 (m, 1H), 1.56 (d, J = 9.9 Hz, 3H), 1.41 (t, J = 7.3 Hz, 1H). | C |
| 2f | 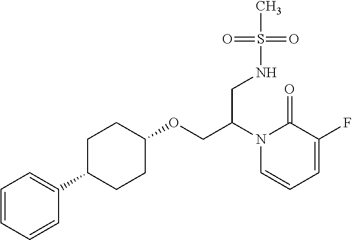 [SMILES: CS(=O)(=O)NCC(CO[C@H]1CC[C@@H](c2ccccc2)CC1)n1cccc(F)c1=O] | N-[2-(3-fluoro- 2-oxo-1,2- dihydropyridin- 1-yl)-3- {[(CIS)-4- phenylcyclo- hexyl] oxy}propyl] methane- sulfonamide | 423.2 [M + H]⁺ | ¹H NMR (400 MHz, CDCl₃) δ 7.51 (dt, J = 7.1, 1.6 Hz, 3H), 7.29 (t, J = 7.5 Hz, 2H), 7.21- 7.10 (m, 4H), 6.17 (td, J = 7.3, 4.6 Hz, 1H), 5.22 (t, J = 6.4 Hz, 1H), 5.15 (q, J = 4.2 Hz, 1H), 3.90 (dd, J = 10.4, 5.3 Hz, 1H), 3.83-3.61 (m, 4H), 2.91 (s, 1H), 2.53 (p, J = 7.8 Hz, 1H), 1.98 (d, J = 8.8 Hz, 3H), 1.64 (qd, J = 9.4, 8.9, 4.1 Hz, 5H). | C |
| 2g | 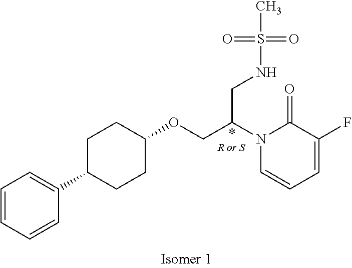 [SMILES: CS(=O)(=O)NCC(CO[C@H]1CC[C@@H](c2ccccc2)CC1)n1cccc(F)c1=O] | (R or S) N-[2- (3-fluoro-2- oxo-1,2- dihydropyridin- 1-yl)-3- {[(CIS)-4- phenylcyclo- hexyl] oxy}propyl] methane- sulfonamide- Isomer 1 | 423.2 [M + H]⁺ | ¹H NMR (400 MHz, CDCl₃) δ 7.50 (dt, J = 7.1, 1.6 Hz, 1H), 7.36-7.27 (m, 2H), 7.24-7.09 (m, 4H), 6.17 (td, J = 7.3, 4.6 Hz, 1H), 5.14 (td, J = 9.0, 5.2 Hz, 1H), 5.07 (s, 1H), 3.91 (dd, J = 10.4, 5.4 Hz, 1H), 3.83-3.61 (m, 4H), 2.92 (s, 3H), 2.53 (p, J = 7.8 Hz, 1H), 2.00 (ddd, J = 14.3, 6.4, 2.9 Hz, 2H), 1.70- 1.55 (m, 6H). | C |
| 2h | 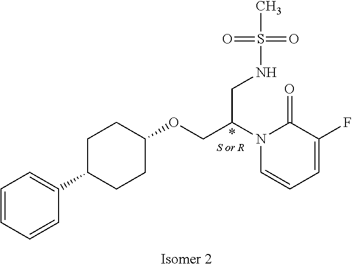 [SMILES: CS(=O)(=O)NCC(CO[C@H]1CC[C@@H](c2ccccc2)CC1)n1cccc(F)c1=O] | (S or R) N-[2- (3-fluoro-2- oxo-1,2- dihydropyridin- 1-yl)-3- {[(CIS)-4- phenylcyclo- hexyl] oxy}propyl] methane- sulfonamide- Isomer 2 | 423.2 [M + H]⁺ | ¹H NMR (400 MHz, CDCl₃) δ 7.50 (dt, J = 7.1, 1.6 Hz, 1H), 7.33-7.26 (m, 2H), 7.23-7.09 (m, 4H), 6.17 (td, J = 7.2, 4.6 Hz, 1H), 5.15 (dd, J = 8.4, 3.7 Hz, 2H), 3.91 (dd, J = 10.4, 5.5 Hz, 1H), 3.79 (dd, J = 10.4, 3.7 Hz, 1H), 3.77-3.66 (m, 2H), 3.66-3.61 (m, 1H), 2.92 (s, 3H), 2.53 (p, J = 7.8 Hz, 1H), 1.99 (ddq, J = 13.9, 8.6, 2.7 Hz, 2H), 1.69-1.57 (m, 6H). | D |
| 2i | 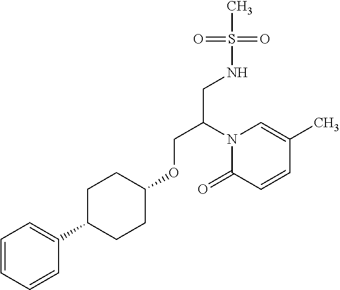 [SMILES: Cc1ccc(=O)n(C(CNS(C)(=O)=O)CO[C@H]2CC[C@@H](c3ccccc3)CC2)c1] | N-[2-(5- methyl-2-oxo- 1,2- dihydropyridin- 1-yl)-3- {[(CIS)-4- phenylcyclo- hexyl] oxy}propyl] methane- sulfonamide | 419.5 [M + H]⁺ | ¹H NMR (400 MHz, CDCl₃) δ 7.42 (d, J = 2.4 Hz, 1H), 7.26 (s, 9H), 6.53 (d, J = 9.2 Hz, 1H), 5.17 (t, J = 6.1 Hz, 1H), 5.08- 5.01 (m, 1H), 3.88 (dd, J = 10.3, 5.5 Hz, 1H), 3.83-3.59 (m, 4H), 2.89 (s, 3H), 2.53 (p, J = 8.0 Hz, 1H), 2.07 (d, J = 1.1 Hz, 3H), 1.56 (m, 5H). | B |
| 2j | 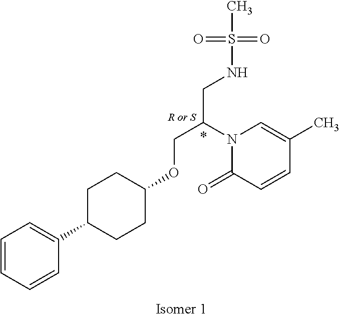 [SMILES: Cc1ccc(=O)n(C(CNS(C)(=O)=O)CO[C@H]2CC[C@@H](c3ccccc3)CC2)c1] | (R or S) N-[2- (5-methyl-2- oxo-1,2- dihydropyridin- 1-yl)-3- {[(CIS)-4- phenylcyclo- hexyl] oxy}propyl] methane- sulfonamide- Isomer 1 | 419.2 [M + H]⁺ | ¹H NMR (400 MHz, CDCl₃) δ 7.44-7.39 (m, 1H), 7.29 (ddd, J = 7.7, 6.4, 1.3 Hz, 2H), 7.24- 7.14 (m, 4H), 6.53 (d, J = 9.2 Hz, 1H), 5.18 (s, 1H), 5.05 (dq, J = 9.2, 5.0 Hz, 1H), 3.88 (dd, J = 10.3, 5.5 Hz, 1H), 3.79 (dd, J = 10.3, 3.8 Hz, 1H), 3.77-3.61 (m, 3H), 2.89 (s, 3H), 2.53 (p, J = 7.8 Hz, 1H), 2.07 (d, J = 1.1 Hz, 3H), 2.01 (ddd, J = 14.1, 6.5, 3.0 Hz, 2H), 1.65 (q, J = 6.5, 5.6 Hz, 4H), 1.55 (s, 2H). | B |
| 2k | 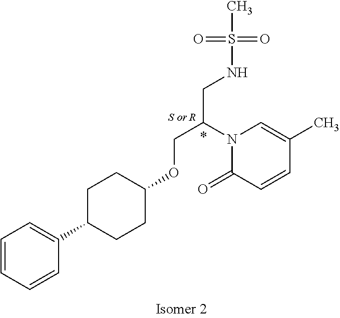 [SMILES: Cc1ccc(=O)n(C(CNS(C)(=O)=O)CO[C@H]2CC[C@@H](c3ccccc3)CC2)c1] | (S or R) N-[2- (5-methyl-2- oxo-1,2- dihydropyridin- 1-yl)-3- {[(CIS)-4- phenylcyclo- hexyl] oxy}propyl] methane- sulfonamide- Isomer 2 | 419.2 [M + H]⁺ | ¹H NMR (400 MHz, CDCl₃) δ 7.43-7.40 (m, 1H), 7.29 (ddd, J = 7.6, 6.4, 1.2 Hz, 2H), 7.24- 7.15 (m, 4H), 6.53 (d, J = 9.2 Hz, 1H), 5.18 (s, 1H), 5.05 (dq, J = 9.0, 5.0 Hz, 1H), 3.88 (dd, J = 10.3, 5.4 Hz, 1H), 3.79 (dd, J = 10.3, 3.9 Hz, 1H), 3.77-3.61 (m, 3H), 2.89 (s, 3H), 2.53 (p, J = 7.7 Hz, 1H), 2.07 (d, J = 1.1 Hz, 3H), 2.05-1.94 (m, 2H), 1.70-1.61 (m, 4H), 1.55 (s, 2H). | D |
| 2l | 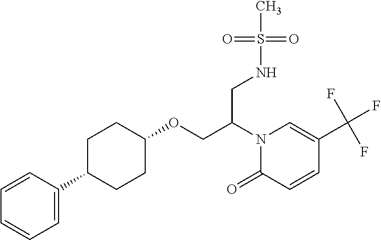 [SMILES: CS(=O)(=O)NCC(CO[C@H]1CC[C@@H](c2ccccc2)CC1)n1cc(C(F)(F)F)ccc1=O] | N-{2-[2-oxo-5- (trifluoro- methyl)-1,2- dihydropyridin- 1-yl]-3- {[(CIS)-4- phenylcyclo- hexyl] oxy}propyl} methane- sulfonamide | 473.2 [M + H]⁺ | ¹H NMR (400 MHz, CDCl₃) δ 8.20 (s, 1H), 7.49 (dd, J = 9.6, 2.6 Hz, 1H), 7.32-7.27 (m, 2H), 7.18 (t, J = 8.9 Hz, 3H), 6.65 (d, J = 9.6 Hz, 1H), 5.12 (s, 1H), 4.96 (t, J = 6.1 Hz, 1H), 3.94 (dd, J = 10.7, 5.0 Hz, 1H), 3.81 (dd, J = 10.7, 3.0 Hz, 1H), 3.74- 3.64 (m, 3H), 2.92 (s, 3H), 2.51 (d, J = 15.2 Hz, 1H), 2.01 (t, J = 13.2 Hz, 2H), 1.71-1.59 (m, 6H). | D |
| 2m | 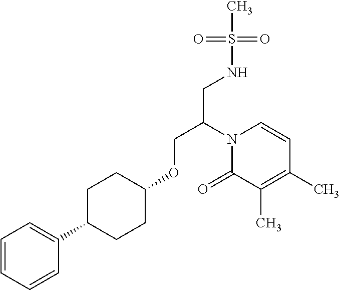 [SMILES: Cc1ccn(C(CNS(C)(=O)=O)CO[C@H]2CC[C@@H](c3ccccc3)CC2)c(=O)c1C] | N-[2-(3,4- dimethyl-2- oxo-1,2- dihydropyridin- 1-yl)-3- {[(CIS)-4- phenylcyclo- hexyl] oxy}propyl] methane- sulfonamide | 433.2 [M + H]⁺ | ¹H NMR (400 MHz, CDCl₃) δ 7.42 (d, J = 7.1 Hz, 1H), 7.31 (t, J = 7.6 Hz, 2H), 7.24-7.15 (m, 3H), 6.11 (d, J = 7.1 Hz, 1H), 5.24 (t, J = 6.1 Hz, 1H), 5.13- 5.03 (m, 1H), 3.90 (dd, J = 10.2, 5.5 Hz, 1H), 3.82-3.62 (m, 4H), 2.91 (s, 3H), 2.62-2.47 (m, 1H), 2.19 (s, 3H), 2.11 (s, 3H), 2.06-1.96 (m, 2H), 1.71- 1.62 (m, 4H), 1.58-1.43 (m, 2H). | B |
| 2n | 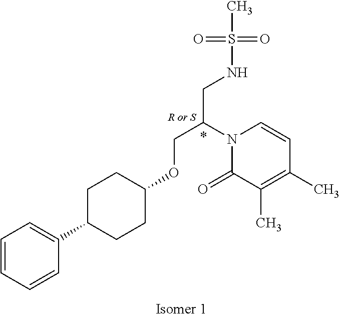 [SMILES: Cc1ccn(C(CNS(C)(=O)=O)CO[C@H]2CC[C@@H](c3ccccc3)CC2)c(=O)c1C] | (R or S) N-[2- (3,4-dimethyl- 2-oxo-1,2- dihydropyridin- 1-yl)-3- {[(CIS)-4- phenylcyclo- hexyl] oxy}propyl] methane- sulfonamide- Isomer 1 | 433.2 [M + H]⁺ | ¹H NMR (400 MHz, CDCl₃) δ 7.48-7.37 (m, 1H), 7.35-7.29 (m, 2H), 7.25-7.13 (m, 3H), 6.11 (d, J = 7.1 Hz, 1H), 5.22- 5.12 (m, 1H), 5.11-4.99 (m, 1H), 3.91 (dd, J = 10.2, 5.5 Hz, 1H), 3.85-3.58 (m, 4H), 2.91 (s, 3H), 2.63-2.43 (m, 1H), 2.19 (s, 3H), 2.11 (s, 3H), 2.08- 1.93 (m, 2H), 1.77-1.61 (m, 4H), 1.56-1.42 (m, 2H). | B |
| 2o | 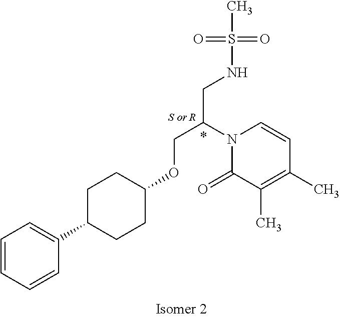 [SMILES: Cc1ccn(C(CNS(C)(=O)=O)CO[C@H]2CC[C@@H](c3ccccc3)CC2)c(=O)c1C] | (S or R) N-[2- (3,4-dimethyl- 2-oxo-1,2- dihydropyridin- 1-yl)-3- {[(CIS)-4- phenylcyclo- hexyl] oxy}propyl] methane- sulfonamide- Isomer 2 | 433.2 [M + H]⁺ | ¹H NMR (400 MHz, CDCl₃) δ 7.42 (d, J = 7.1 Hz, 1H), 7.37- 7.29 (m, 2H), 7.25-7.15 (m, 3H), 6.11 (d, J = 7.1 Hz, 1H), 5.28-5.13 (m, 1H), 5.11-5.00 (m, 1H), 3.91 (dd, J = 10.2, 5.5 Hz, 1H), 3.84-3.61 (m, 4H), 2.91 (s, 3H), 2.62-2.46 (m, 1H), 2.19 (s, 3H), 2.11 (s, 3H), 2.07-1.93 (m, 2H), 1.75-1.61 (m, 4H), 1.56-1.45 (m, 2H). | D |
| 2p | 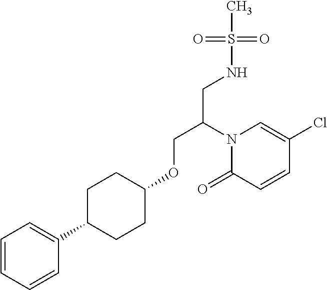 [SMILES: CS(=O)(=O)NCC(CO[C@H]1CC[C@@H](c2ccccc2)CC1)n1cc(Cl)ccc1=O] | N-[2-(5-chloro- 2-oxo-1,2- dihydropyridin- 1-yl)-3- {[(CIS)-4- phenylcyclo- hexyl] oxy}propyl] methane- sulfonamide | 439.1 [M + H]⁺ | ¹H NMR (400 MHz, CDCl₃) δ 7.85 (d, J = 2.8 Hz, 1H), 7.38- 7.30 (m, 3H), 7.26-7.16 (m, 3H), 6.58 (d, J = 9.7 Hz, 1H), 5.16-4.96 (m, 2H), 3.97-3.77 (m, 2H), 3.76-3.61 (m, 3H), 2.94 (s, 3H), 2.65-2.49 (m, 1H), 2.04 (d, J = 13.6 Hz, 2H), 1.78-1.61 (m, 6H). | C |
| 2q | 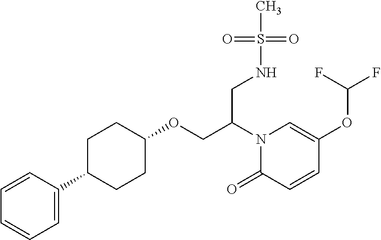 [SMILES: CS(=O)(=O)NCC(CO[C@H]1CC[C@@H](c2ccccc2)CC1)n1cc(OC(F)F)ccc1=O] | N-{2-[5- (difluoro- methoxy)- 2-oxo-1,2- dihydropyridin- 1-yl]-3- {[(CIS)-4- phenylcyclo- hexyl] oxy}propyl} methane- sulfonamide | 471.2 [M + H]⁺ | ¹H NMR (400 MHz, CDCl₃) δ 7.79 (d, J = 3.05 Hz, 1H), 7.34- 7.27 (m, 3H), 7.20 (d, J = 7.39 Hz, 3H), 6.57 (dd, J = 9.79, 0.56 Hz, 1H), 6.36 (t, J = 72.81 Hz, 1H), 5.22 (d, J = 5.65 Hz, 1H), 5.14 (dtd, J = 8.07, 5.11, 3.05 Hz, 1H), 3.89 (dd, J = 10.63, 4.89 Hz, 1H), 3.80 (dd, J = 10.64, 3.08 Hz, 1H), 3.75-3.62 (m, 3H), 2.90 (s, 3H), 2.60- 2.48 (m, 1H), 2.08-1.96 (m, 2H), 1.68 (tdd, J = 8.54, 4.93, 2.76 Hz, 3H), 1.59 (dt, J = 13.65, 4.25 Hz, 3H). | C |
| 2r | 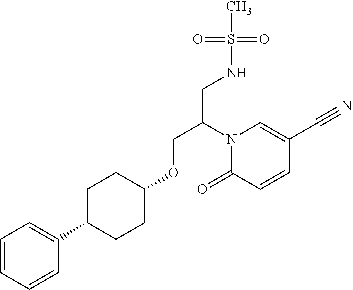 [SMILES: CS(=O)(=O)NCC(CO[C@H]1CC[C@@H](c2ccccc2)CC1)n1cc(C#N)ccc1=O] | N-[2-(5-cyano- 2-oxo-1,2- dihydropyridin- 1-yl)-3- {[(CIS)-4- phenylcyclo- hexyl] oxy}propyl] methane- sulfonamide | 430.5 [M + H]⁺ | ¹H NMR (400 MHz, CDCl₃) δ 8.27 (dd, J = 2.5, 0.6 Hz, 1H), 7.45 (dd, J = 9.5, 2.5 Hz, 1H), 7.38-7.30 (m, 2H), 7.26-7.16 (m, 3H), 6.62 (dd, J = 9.5, 0.6 Hz, 1H), 5.16-5.04 (m, 1H), 4.88 (t, J = 6.4 Hz, 1H), 3.95 (dd, J = 10.7, 5.3 Hz, 1H), 3.81 (dd, J = 10.7, 3.2 Hz, 1H), 3.75- 3.63 (m, 3H), 2.96 (s, 3H), 2.63-2.49 (m, 1H), 2.09-1.96 (m, 2H), 1.82-1.58 (m, 6H). | C |
| 2s | 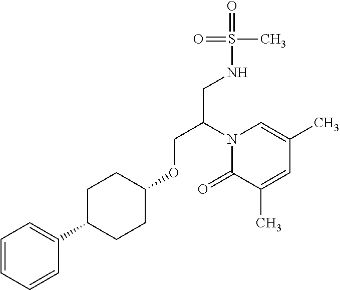 [SMILES: Cc1cc(C)c(=O)n(C(CNS(C)(=O)=O)CO[C@H]2CC[C@@H](c3ccccc3)CC2)c1] | N-[2-(3,5- dimethyl-2- oxo-1,2- dihydropyridin- 1-yl)-3- {[(CIS)-4- phenylcyclo- hexyl] oxy}propyl] methane- sulfonamide | 433.3 [M + H]⁺ | ¹H NMR (400 MHz, CDCl₃) δ 7.30 (s, 3H), 7.24-7.16 (m, 3H), 7.14-7.11 (m, 1H), 5.21 (s, 1H), 5.12-5.03 (m, 1H), 3.92-3.85 (m, 1H), 3.83-3.77 (m, 1H), 3.77-3.66 (m, 2H), 3.66-3.63 (m, 1H), 2.90 (s, 3H), 2.59-2.48 (m, 1H), 2.14 (s, 3H), 2.09-2.05 (m, 3H), 2.05-1.97 (m, 2H), 1.72-1.62 (m, 4H), 1.57-1.49 (m, 2H). | A |
| 2t | 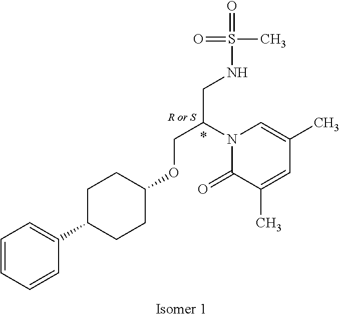 [SMILES: Cc1cc(C)c(=O)n(C(CNS(C)(=O)=O)CO[C@H]2CC[C@@H](c3ccccc3)CC2)c1] | (R or S) N-[2- (3,5-dimethyl- 2-oxo-1,2- dihydropyridin- 1-yl)-3- {[(CIS)-4- phenylcyclo- hexyl] oxy}propyl] methane- sulfonamide- Isomer 1 | 433.2 [M + H]⁺ | ¹H NMR (400 MHz, CDCl₃) δ 7.30 (d, J = 7.5 Hz, 3H), 7.20 (d, J = 1.5 Hz, 3H), 7.15-7.11 (m, 1H), 5.17 (s, 1H), 5.12-5.03 (m, 1H), 3.93-3.78 (m, 2H), 3.78-3.68 (m, 2H), 3.67-3.64 (m, 1H), 2.91 (s, 3H), 2.59- 2.50 (m, 1H), 2.15 (s, 3H), 2.07 (d, J = 0.9 Hz, 3H), 2.06-1.98 (m, 2H), 1.72-1.63 (m, 4H), 1.63-1.58 (m, 1H), 1.57-1.51 (m, 1H). | A |
| 2u | 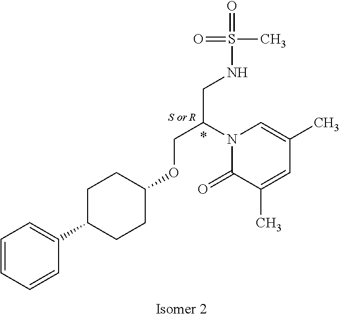 [SMILES: Cc1cc(C)c(=O)n(C(CNS(C)(=O)=O)CO[C@H]2CC[C@@H](c3ccccc3)CC2)c1] | (S or R) N-[2- (3,5-dimethyl- 2-oxo-1,2- dihydropyridin- 1-yl)-3- {[(CIS)-4- phenylcyclo- hexyl] oxy}propyl] methane- sulfonamide- Isomer 2 | 433.2 [M + H]⁺ | ¹H NMR (400 MHz, CDCl₃) δ 7.34-7.29 (m, 3H), 7.20 (d, J = 1.54 Hz, 3H), 7.15-7.09 (m, 1H), 5.19-5.13 (m, 1H), 5.12- 5.03 (m, 1H), 3.94-3.79 (m, 2H), 3.70 (s, 2H), 3.67-3.64 (m, 1H), 2.91 (s, 3H), 2.54 (s, 1H), 2.15 (s, 3H), 2.07 (d, J = 0.88 Hz, 3H), 2.06-1.97 (m, 2H), 1.74-1.63 (m, 4H), 1.57- 1.50 (m, 2H). | C |
| 2v | 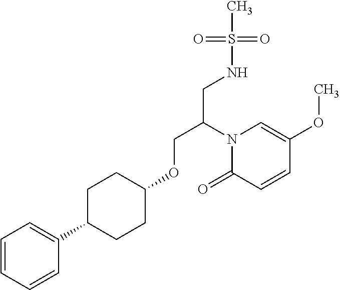 [SMILES: COc1ccc(=O)n(C(CNS(C)(=O)=O)CO[C@H]2CC[C@@H](c3ccccc3)CC2)c1] | N-[2-(5- methoxy-2- oxo-1,2- dihydropyridin- 1-yl)-3- {[(CIS)-4- phenylcyclo- hexyl] oxy}propyl] methane- sulfonamide | 435.2 [M + H]⁺ | ¹H NMR (400 MHz, CDCl₃) δ 7.35-7.29 (m, 3H), 7.26-7.16 (m, 4H), 6.58 (dd, J = 9.8, 0.6 Hz, 1H), 5.24 (t, J = 6.0 Hz, 1H), 5.13 (dq, J = 8.7, 4.6 Hz, 1H), 3.93 (dd, J = 10.5, 5.2 Hz, 1H), 3.87-3.75 (m, 2H), 3.73-3.62 (m, 5H), 2.92 (s, 3H), 2.62- 2.49 (m, 1H), 2.13-1.95 (m, 2H), 1.82-1.60 (m, 6H). | C |
| 2w | 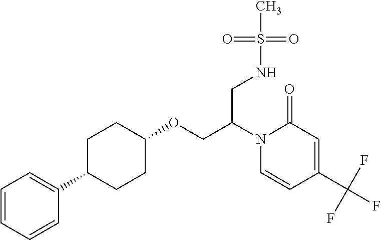 [SMILES: CS(=O)(=O)NCC(CO[C@H]1CC[C@@H](c2ccccc2)CC1)n1ccc(C(F)(F)F)cc1=O] | N-{2-[2-oxo-4- (trifluoro- methyl)-1,2- dihydropyridin- 1-yl]-3- {[(CIS)-4- phenylcyclo- hexyl] oxy}propyl} methane- sulfonamide | 473.2 [M + H]⁺ | ¹H NMR (500 MHz, CDCl₃) δ 7.83 (d, J = 7.3 Hz, 1H), 7.28- 7.33 (m, 2H), 7.18-7.24 (m, 1H), 7.16 (d, J = 7.1 Hz, 2H), 6.86 (s, 1H), 6.39 (dd, J = 7.3, 1.9 Hz, 1H), 5.05-5.16 (m, 1H), 5.01 (br t, J = 6.2 Hz, 1H), 3.92 (dd, J = 10.4, 5.4 Hz, 1H), 3.72- 3.81 (m, 2H), 3.68 (dt, J = 13.9, 5.6 Hz, 2H), 2.89-2.98 (m, 3H), 2.47-2.60 (m, 1H), 1.94-2.04 (m, 2H), 1.45-1.74 (m, 6H). | D |
| 2x | 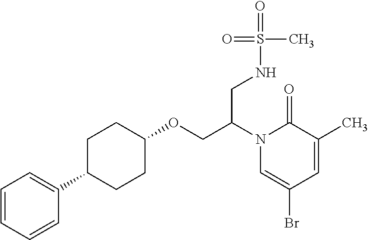 [SMILES: Cc1cc(Br)cn(C(CNS(C)(=O)=O)CO[C@H]2CC[C@@H](c3ccccc3)CC2)c1=O] | N-[2-(5- bromo-3- methyl-2-oxo- 1,2- dihydropyridin- 1-yl)-3- {[(CIS)-4- phenylcyclo- hexyl] oxy}propyl] methane- sulfonamide | 499.1 [M + H]⁺ | ¹H NMR (400 MHz, CDCl₃) δ 7.79 (dd, J = 2.7, 0.9 Hz, 1H), 7.35-7.29 (m, 3H), 7.27-7.18 (m, 3H), 5.11 (q, J = 8.0 Hz, 1H), 5.04 (t, J = 6.1 Hz, 1H), 3.97-3.77 (m, 2H), 3.75-3.63 (m, 3H), 2.94 (s, 3H), 2.55 (p, J = 7.8 Hz, 1H), 2.17 (t, J = 0.9 Hz, 3H), 2.11-1.98 (m, 2H), 1.78-1.59 (m, 6H) | B |
| 2y | 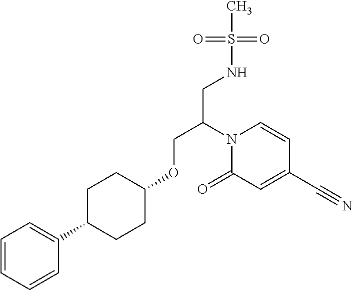 [SMILES: CS(=O)(=O)NCC(CO[C@H]1CC[C@@H](c2ccccc2)CC1)n1ccc(C#N)cc1=O] | N-[2-(4-cyano- 2-oxo-1,2- dihydropyridin- 1-yl)-3- {[(CIS)-4- phenylcyclo- hexyl] oxy}propyl] methane- sulfonamide | 430.2 [M + H]⁺ | ¹H NMR (400 MHz, CDCl₃) δ 7.83 (d, J = 7.1 Hz, 1H), 7.39- 7.31 (m, 2H), 7.27-7.11 (m, 3H), 6.96-6.92 (m, 1H), 6.36 (dd, J = 7.2, 1.9 Hz, 1H), 5.13- 5.01 (m, 1H), 4.91 (t, J = 6.3 Hz, 1H), 3.93 (dd, J = 10.5, 5.4 Hz, 1H), 3.84-3.73 (m, 2H), 3.72- 3.60 (m, 2H), 2.96 (s, 3H), 2.65- 2.46 (m, 1H), 2.01 (t, J = 10.9 Hz, 2H), 1.78-1.59 (m, 6H). | D |
| 2z | 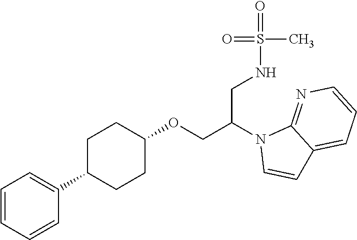 [SMILES: CS(=O)(=O)NCC(CO[C@H]1CC[C@@H](c2ccccc2)CC1)n1ccc2cccnc21] | N-(2-{1H- pyrrolo[2,3- b]pyridin-1- yl}-3-{[(CIS)- 4- phenylcyclo- hexyl] oxy}propyl) methane- sulfonamide | 428.48 [M + H]⁺ | ¹H NMR (400 MHz, CDCl₃) δ 8.29 (dd, J = 4.73, 1.43 Hz, 1H), 7.95 (dd, J = 7.81, 1.43 Hz, 1H), 7.50 (d, J = 3.52 Hz, 1H), 7.28- 7.36 (m, 2H), 7.23-7.14 (m, 3H), 7.10 (dd, J = 7.81, 4.73 Hz, 1H), 6.53 (d, J = 3.52 Hz, 1H), 5.77 (t, J = 5.72 Hz, 1H), 5.19- 5.05 (m, 1H), 4.04-3.95 (m, 1H), 3.95-3.86 (m, 2H), 3.84- 3.74 (m, 1H), 3.64 (br. s., 1H), 2.74-2.66 (m, 3H), 2.49 (d, J = 3.08 Hz, 1H), 2.05-1.91 (m, 2H), 1.77-1.58 (m, 3H), 1.56- 1.42 (m, 3H). | C |
| 2aa | 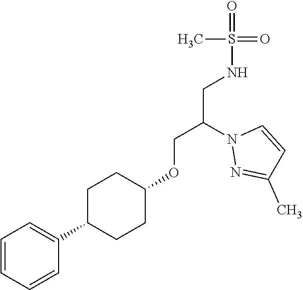 [SMILES: Cc1ccn(C(CNS(C)(=O)=O)CO[C@H]2CC[C@@H](c3ccccc3)CC2)n1] | N-[2-(3- methyl-1H- pyrazol-1-yl)- 3-{[(CIS)-4- phenylcyclo- hexyl] oxy}propyl] methane- sulfonamide | 392.3 [M + H]⁺ | ¹H NMR (400 MHz, CDCl₃) δ 7.44 (dd, J = 10.9, 2.0 Hz, 1H), 7.32-7.26 (m, 2H), 7.21-7.14 (m, 3H), 6.04 (dd, J = 5.5, 1.6 Hz, 1H), 5.26 (dt, J = 20.4, 6.3 Hz, 1H), 4.63-4.39 (m, 1H), 3.81-3.62 (m, 4H), 3.61-3.51 (m, 1H), 2.91 (d, J = 7.0 Hz, 3H), 2.54-2.41 (m, 1H), 2.32 (d, J = 46.9 Hz, 3H), 2.03-1.90 (m, 2H), 1.75-1.39 (m, 6H). | D |
| 2ab | 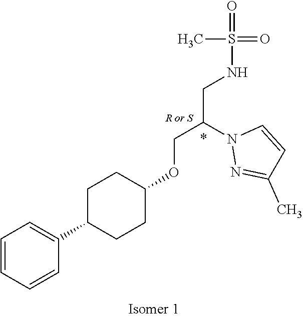 [SMILES: Cc1ccn(C(CNS(C)(=O)=O)CO[C@H]2CC[C@@H](c3ccccc3)CC2)n1] | (R or S) N-[2- (3-methyl-1H- pyrazol-1-yl)- 3-{[(CIS)-4- phenylcyclo- hexyl] oxy}propyl] methane- sulfonamide- Isomer 1 | 392.2 [M + H]⁺ | ¹H NMR (400 MHz, CDCl₃) δ 7.45 (d, J = 2.3 Hz, 1H), 7.33- 7.27 (m, 2H), 7.18 (td, J = 5.6, 3.0 Hz, 3H), 6.05 (d, J = 2.3 Hz, 1H), 5.24 (t, J = 6.2 Hz, 1H), 4.51-4.40 (m, 1H), 3.81-3.63 (m, 4H), 3.59 (t, J = 3.0 Hz, 1H), 2.90 (s, 3H), 2.49 (td, J = 13.6, 11.8, 4.3 Hz, 1H), 2.26 (s, 3H), 2.04-1.90 (m, 2H), 1.78-1.40 (m, 6H). | D |
| 2ac | 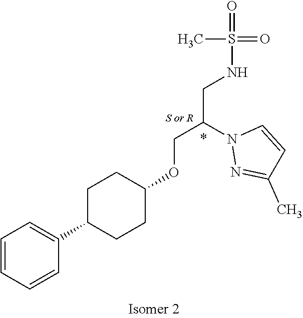 [SMILES: Cc1ccn(C(CNS(C)(=O)=O)CO[C@H]2CC[C@@H](c3ccccc3)CC2)n1] | (S or R) N-[2- (3-methyl-1H- pyrazol-1-yl)- 3-{[(CIS)-4- phenylcyclo- hexyl] oxy}propyl] methane- sulfonamide- Isomer 2 | 392.2 [M + H]⁺ | ¹H NMR (400 MHz, CDCl₃) δ 7.45 (d, J = 2.2 Hz, 1H), 7.33- 7.26 (m, 2H), 7.22-7.15 (m, 3H), 6.04 (d, J = 2.2 Hz, 1H), 5.24 (t, J = 6.1 Hz, 1H), 4.45 (qd, J = 6.4, 4.1 Hz, 1H), 3.81- 3.63 (m, 4H), 3.58 (q, J = 2.9 Hz, 1H), 2.90 (s, 3H), 2.49 (td, J = 12.3, 10.9, 4.2 Hz, 1H), 2.26 (s, 3H), 1.97 (ddt, J = 17.2, 14.0, 3.1 Hz, 2H), 1.78-1.39 (m, 6H). | D |
| 2ad | 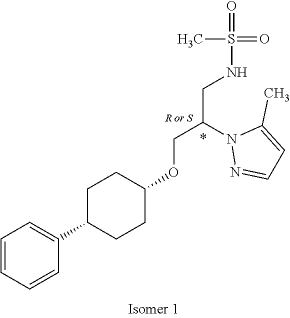 [SMILES: Cc1ccnn1C(CNS(C)(=O)=O)CO[C@H]1CC[C@@H](c2ccccc2)CC1] | (R or S) N-[2- (5-methyl-1H- pyrazol-1-yl)- 3-{[(CIS)-4- phenylcyclo- hexyl] oxy}propyl] methane- sulfonamide- Isomer 1 | 392.2 [M + H]⁺ | ¹H NMR (400 MHz, CDCl₃) δ 7.42 (d, J = 2.0 Hz, 1H), 7.29 (d, J = 7.6 Hz, 2H), 7.22-7.12 (m, 3H), 6.06-6.01 (m, 1H), 5.29 (t, J = 6.3 Hz, 1H), 4.62-4.52 (m, 1H), 3.82-3.68 (m, 3H), 3.62 (ddd, J = 13.2, 6.1, 4.0 Hz, 1H), 3.54 (d, J = 3.9 Hz, 1H), 2.92 (s, 3H), 2.47 (d, J = 11.8 Hz, 1H), 2.38 (s, 3H), 2.02- 1.93 (m, 1H), 1.89-1.81 (m, 1H), 1.76-1.35 (m, 6H). | D |
| 2ae | 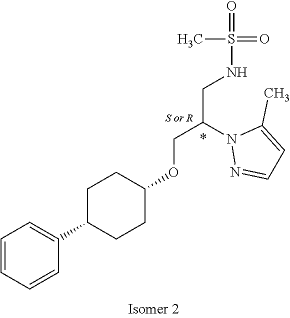 [SMILES: Cc1ccnn1C(CNS(C)(=O)=O)CO[C@H]1CC[C@@H](c2ccccc2)CC1] | (S or R) N-[2- (5-methyl-1H- pyrazol-1-yl)- 3-{[(CIS)-4- phenylcyclo- hexyl] oxy}propyl] methane- sulfonamide- Isomer 2 | 392.2 [M + H]⁺ | ¹H NMR (400 MHz, CDCl₃) δ 7.42 (d, J = 1.8 Hz, 1H), 7.29 (d, J = 7.5 Hz, 2H), 7.22-7.12 (m, 3H), 6.03 (d, J = 1.7 Hz, 1H), 5.29 (t, J = 6.4 Hz, 1H), 4.57 (q, J = 10.7, 9.0 Hz, 1H), 3.81- 3.68 (m, 3H), 3.62 (ddd, J = 13.2, 6.1, 3.9 Hz, 1H), 3.54 (s, 1H), 2.92 (s, 3H), 2.52-2.41 (m, 1H), 2.38 (s, 3H), 1.97 (dt, J = 13.8, 3.1 Hz, 1H), 1.86 (d, J = 11.7 Hz, 1H), 1.74-1.36 (m, 6H). | D |
| 2af | 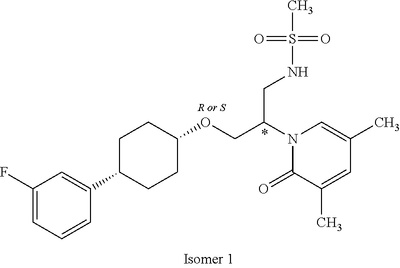 [SMILES: Cc1cc(C)c(=O)n(C(CNS(C)(=O)=O)CO[C@H]2CC[C@@H](c3cccc(F)c3)CC2)c1] | (R or S) N-[2- (3,5-dimethyl- 2-oxo-1,2- dihydropyridin- 1-yl)-3- {[(CIS)-4-(3- fluorophenyl) cyclohexyl] oxy}propyl] methane- sulfonamide- Isomer 1 | 451.3 [M + H]⁺ | ¹H NMR (500 MHz, CDCl₃) δ 7.26-7.21 (m, 2H), 7.14 (d, J = 1.1 Hz, 1H), 6.95 (d, J = 7.7 Hz, 1H), 6.91-6.86 (m, 1H), 6.84 (dt, J = 10.4, 2.0 Hz, 1H), 5.21 (br t, J = 6.0 Hz, 1H), 5.10-4.91 (m, 1H), 3.89 (dd, J = 10.2, 5.7 Hz, 1H), 3.78 (dd, J = 10.2, 4.0 Hz, 1H), 3.76-3.70 (m, 1H), 3.70-3.61 (m, 2H), 2.90 (s, 3H), 2.58-2.47 (m, 1H), 2.13 (s, 3H), 2.06 (d, J = 0.7 Hz, 3H), 2.04-1.95 (m, 2H), 1.67-1.48 (m, 6H). | A |
| 2ag | 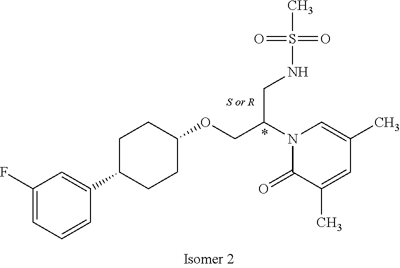 [SMILES: Cc1cc(C)c(=O)n(C(CNS(C)(=O)=O)CO[C@H]2CC[C@@H](c3cccc(F)c3)CC2)c1] | (S or R) N-[2- (3,5-dimethyl- 2-oxo-1,2- dihydropyridin- 1-yl)-3- {[(CIS)-4-(3- fluorophenyl) cyclohexyl] oxy}propyl] methane- sulfonamide- Isomer 2 | 451.3 [M + H]⁺ | ¹H NMR (500 MHz, CDCl₃) δ 7.26-7.20 (m, 2H), 7.16-7.10 (m, 1H), 6.94 (d, J = 7.7 Hz, 1H), 6.91-6.86 (m, 1H), 6.83 (dt, J = 10.4, 2.1 Hz, 1H), 5.22 (br t, J = 6.0 Hz, 1H), 5.12-4.93 (m, 1H), 3.89 (dd, J = 10.2, 5.8 Hz, 1H), 3.78 (dd, J = 10.2, 4.1 Hz, 1H), 3.76-3.71 (m, 1H), 3.69-3.62 (m, 2H), 2.90 (s, 3H), 2.57-2.48 (m, 1H), 2.13 (s, 3H), 2.06 (d, J = 0.7 Hz, 3H), 2.04-1.94 (m, 2H), 1.67-1.48 (m, 6H). | C |
| 2ah | 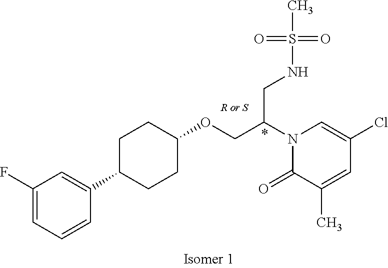 [SMILES: Cc1cc(Cl)cn(C(CNS(C)(=O)=O)CO[C@H]2CC[C@@H](c3cccc(F)c3)CC2)c1=O] | (R or S) N-[2- (5-chloro-3- methyl-2-oxo- 1,2- dihydropyridin- 1-yl)-3- {[(CIS)-4-(3- fluorophenyl) cyclohexyl] oxy}propyl] methane- sulfonamide- Isomer 1 | 471.2 [M + H]⁺ | ¹H NMR (400 MHz, CDCl₃) δ 7.68 (d, J = 2.4 Hz, 1H), 7.26- 7.19 (m, 2H), 6.98 (d, J = 7.7 Hz, 1H), 6.95-6.83 (m, 2H), 5.16-5.05 (m, 2H), 3.95-3.84 (m, 1H), 3.84-3.76 (m, 1H), 3.74-3.61 (m, 3H), 2.93 (s, 3H), 2.62-2.45 (m, 1H), 2.15 (s, 3H), 2.09-1.94 (m, 2H), 1.75-1.48 (m, 6H). | A |
| 2ai | 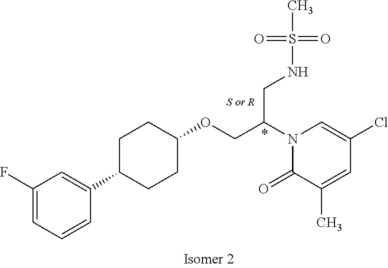 [SMILES: Cc1cc(Cl)cn(C(CNS(C)(=O)=O)CO[C@H]2CC[C@@H](c3cccc(F)c3)CC2)c1=O] | (S or R) N-[2- (5-chloro-3- methyl-2-oxo- 1,2- dihydropyridin- 1-yl)-3- {[(CIS)-4-(3- fluorophenyl) cyclohexyl] oxy}propyl] methane- sulfonamide- Isomer 2 | 471.2 [M + H]⁺ | ¹H NMR (400 MHz, CDCl₃) δ 7.68 (d, J = 2.4 Hz, 1H), 7.26- 7.19 (m, 2H), 6.98 (d, J = 7.7 Hz, 1H), 6.95-6.83 (m, 2H), 5.16-5.05 (m, 2H), 3.95-3.84 (m, 1H), 3.84-3.76 (m, 1H), 3.74-3.61 (m, 3H), 2.93 (s, 3H), 2.62-2.45 (m, 1H), 2.15 (s, 3H), 2.09-1.94 (m, 2H), 1.75-1.48 (m, 6H). | C |
| 2aj | 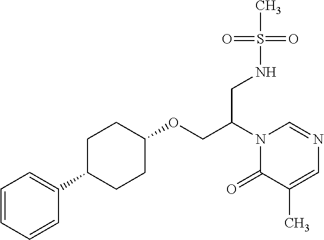 [SMILES: Cc1cncn(C(CNS(C)(=O)=O)CO[C@H]2CC[C@@H](c3ccccc3)CC2)c1=O] | N-[2-(5- methyl-6-oxo- 1,6-dihydro- pyrimidin- 1-yl)-3- {[(CIS)-4- phenylcyclo- hexyl] oxy}propyl] methane- sulfonamide | 420.2 [M + H]⁺ | ¹H NMR (400 MHz, CDCl₃) δ 8.31 (s, 1H), 7.83 (d, J = 0.9 Hz, 1H), 7.35-7.29 (m, 2H), 7.24- 7.16 (m, 3H), 5.04 (s, 1H), 4.94- 4.85 (m, 1H), 3.96 (d, J = 5.9 Hz, 1H), 3.87-3.76 (m, 2H), 3.72 (s, 1H), 3.67 (br. s., 1H), 2.95 (s, 3H),2.58-2.48 (m, 1H), 2.09 (s, 3H), 2.01 (d, J = 13.4 Hz, 2H), 1.72-1.58 (m, 5H), 1.55-1.47 (m, 1H). | D |
| 2al | 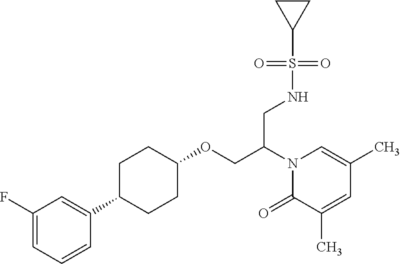 [SMILES: Cc1cc(C)c(=O)n(C(CNS(=O)(=O)C2CC2)CO[C@H]2CC[C@@H](c3cccc(F)c3)CC2)c1] | N-[2-(3,5- dimethyl-2- oxo-1,2- dihydropyridin- 1-yl)-3- {[(CIS)-4-(3- fluorophenyl) cyclohexyl] oxy} propyl]cyclo- propane- sulfonamide | 477.4 [M + H]+ | ¹H NMR (400 MHz, CDCl₃) δ 7.26-7.20 (m, 2H), 7.13 (s, 1H), 6.95 (d, J = 7.7 Hz, 1H), 6.92-6.80 (m, 2H), 5.18-4.95 (m, 2H), 3.89 (dd, J = 10.2, 5.6 Hz, 1H), 3.82-3.62 (m, 4H), 2.59-2.46 (m, 1H), 2.38 (tt, J = 8.1, 4.8 Hz, 1H), 2.14 (s, 3H), 2.06 (s, 3H), 2.05-1.93 (m, 2H), 1.72-1.44 (m, 6H), 1.20- 1.11 (m, 2H), 1.00-0.90 (m, 2H). | A |
| 2am | 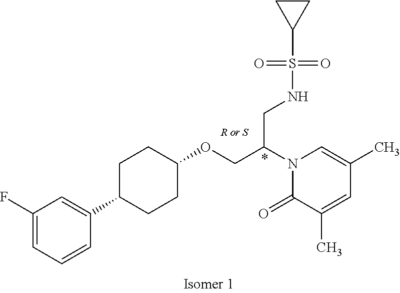 [SMILES: Cc1cc(C)c(=O)n(C(CNS(=O)(=O)C2CC2)CO[C@H]2CC[C@@H](c3cccc(F)c3)CC2)c1] | (R or S) N-[2- (3,5-dimethyl- 2-oxo-1,2- dihydropyridin- 1-yl)-3- {[(CIS)-4-(3- fluorophenyl) cyclohexyl] oxy} propyl]cyclo- propane- sulfossSn amide-Isomer 1 | 477.4 [M + H]+ | ¹H NMR (500 MHz, CDCl₃) δ 7.27-7.21 (m, 2H), 7.15-7.11 (m, 1H), 6.95 (d, J = 7.7 Hz, 1H), 6.90-6.85 (m, 1H), 6.82- 6.85 (m, 1H), 5.14 (br t, J = 5.9 Hz, 1H), 5.11-5.03 (m, 1H), 3.89 (dd, J = 10.2, 5.6 Hz, 1H), 3.78 (dd, J = 10.2, 4.3 Hz, 1H), 3.77-3.65 (m, 2H), 3.66-3.62 (m, 1H), 2.59-2.47 (m, 1H), 2.38 (tt, J = 8.0, 4.8 Hz, 1H), 2.13 (s, 3H), 2.06 (s, 3H), 2.04- 1.96 (m, 2H), 1.68-1.47 (m, 6H), 1.20-1.10 (m, 2H), 1.02- 0.90 (m, 2H). | |
| 2an | 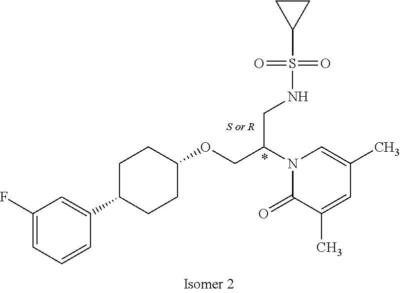 [SMILES: Cc1cc(C)c(=O)n(C(CNS(=O)(=O)C2CC2)CO[C@H]2CC[C@@H](c3cccc(F)c3)CC2)c1] | (S or R) N-[2- (3,5-dimethyl- 2-oxo-1,2- dihydropyridin- 1-yl)-3- {[(CIS)-4-(3- fluorophenyl) cyclohexyl] oxy} propyl]cyclo- propane- sulfonamide- Isomer 2 | 477.4 [M + H]+ | ¹H NMR (500 MHz, CDCl₃) δ 7.26-7.21 (m, 2H), 7.15-7.11 (m, 1H), 6.94 (d, J = 7.7 Hz, 1H), 6.90-6.85 (m, 1H), 6.85- 6.81 (m, 1H), 5.18 (br t, J = 5.9 Hz, 1H), 5.13-5.03 (m, 1H), 3.89 (dd, J = 10.2, 5.6 Hz, 1H), 3.78 (dd, J = 10.1, 4.2 Hz, 1H), 3.77-3.65 (m, 2H), 3.65-3.63 (m, 1H), 2.61-2.45 (m, 1H), 2.38 (tt, J = 8.0, 4.8 Hz, 1H), 2.13 (s, 3H), 2.05 (s, 3H), 2.04- 1.96 (m, 2H), 1.67-1.47 (m, 6H), 1.19-1.11 (m, 2H), 1.02- 0.92 (m, 2H). | C |
| 2ao | 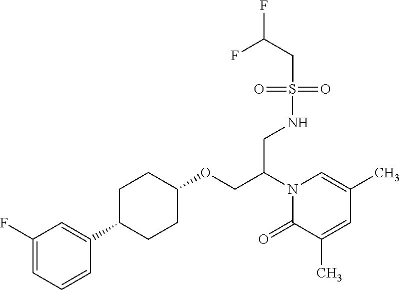 [SMILES: Cc1cc(C)c(=O)n(C(CNS(=O)(=O)CC(F)F)CO[C@H]2CC[C@@H](c3cccc(F)c3)CC2)c1] | N-[2-(3,5- dimethyl-2- oxo-1,2- dihydropyridin- 1-yl)-3- {[(CIS)-4-(3- fluorophenyl) cyclohexyl] oxy} propyl]-2,2- difluoroethane- 1-sulfonamide | 501.3 [M + H]+ | ¹H NMR (400 MHz, CDCl₃) δ 7.32-7.26 (m, 1H), 7.26-7.19 (m, 1H), 7.15 (s, 1H), 6.94 (d, J = 7.7 Hz, 1H), 6.91-6.79 (m, 2H), 6.14-6.10 (m, 1H), 6.14 (tt, J = 55.0, 4.6 Hz, 1H), 5.16- 4.90 (m, 1H), 3.89 (dd, J = 10.3, 5.5 Hz, 1H), 3.79 (dd, J = 10.3, 3.9 Hz, 1H), 3.76-3.62 (m, 3H), 3.58-3.44 (m, 2H), 2.59- 2.44 (m, 1H), 2.13 (s, 3H), 2.06 (s, 3H), 2.05-1.94 (m, 2H), 1.86-1.45 (m, 6H). | B |
| 2ap | 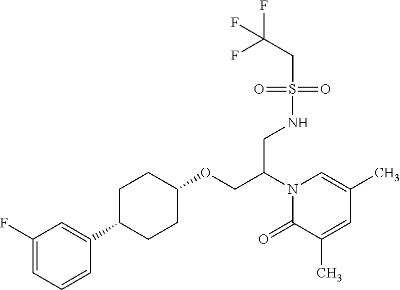 [SMILES: Cc1cc(C)c(=O)n(C(CNS(=O)(=O)CC(F)(F)F)CO[C@H]2CC[C@@H](c3cccc(F)c3)CC2)c1] | N-[2-(3,5- dimethyl-2- oxo-1,2- dihydropyridin- 1-yl)-3- {[(CIS)-4-(3- fluorophenyl) cyclohexyl] oxy} propyl]-2,2,2- trifluoroethane- 1-sulfonamide | 519.3 [M + H]+ | ¹H NMR (400 MHz, CDCl₃) δ 7.29-7.21 (m, 2H), 7.15 (s, 1H), 6.94 (d, J = 7.7 Hz, 1H), 6.91- 6.80 (m, 2H), 6.37-6.08 (m, 1H), 5.18-4.79 (m, 1H), 3.91 (dd, J = 10.3, 5.7 Hz, 1H), 3.85- 3.67 (m, 5H), 3.67-3.62 (m, 1H), 2.52 (td, J = 9.9, 5.3 Hz, 1H), 2.13 (s, 3H), 2.06 (s, 3H), 2.05- 1.94 (m, 2H), 1.71-1.46 (m, 6H). | B |
| 2aq | 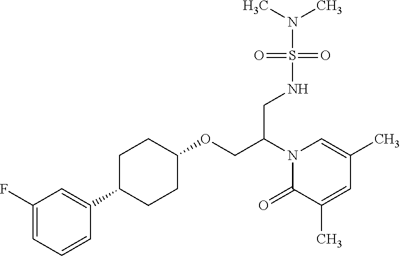 [SMILES: Cc1cc(C)c(=O)n(C(CNS(=O)(=O)N(C)C)CO[C@H]2CC[C@@H](c3cccc(F)c3)CC2)c1] | 1-{1- [(dimethyl- sulfamoyl) amino]- 3-{[(CIS)-4-(3- fluorophenyl) cyclohexyl] oxy} propan-2-yl}- 3,5-dimethyl- 1,2- dihydropyridin- 2-one | 480.3 [M + H]⁺ | ¹H NMR (400 MHz, CDCl₃) δ 7.26-7.19 (m, 2H), 7.13 (br s, 1H), 6.94 (d, J = 7.7 Hz, 1H), 6.91-6.79 (m, 2H), 5.04 (br t, J = 5.9 Hz, 2H), 3.93-3.83 (m, 1H), 3.77 (dd, J = 10.1, 4.2 Hz, 1H), 3.72-3.52 (m, 3H), 2.75 (s, 6H), 2.57-2.46 (m, 1H), 2.13 (s, 3H), 2.06 (s, 3H), 2.04-1.93 (m, 2H), 1.72- 1.43 (m, 6H). | A |
| 2ar | 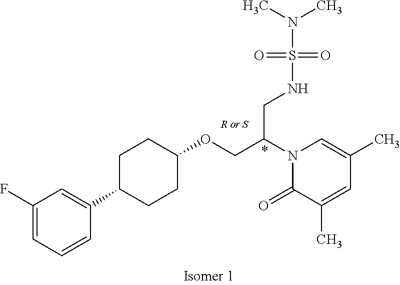 [SMILES: Cc1cc(C)c(=O)n(C(CNS(=O)(=O)N(C)C)CO[C@H]2CC[C@@H](c3cccc(F)c3)CC2)c1] | (R or S) 1-[1- [(dimethyl- sulfamoyl) amino]-3- {[(CIS)-4-(3- fluorophenyl) cyclohexyl] oxy} propan-2-yl]- 3,5-dimethyl- 1,2- dihydropyridin- 2-one- Isomer 1 | 480.2 [M + H]⁺ | ¹H NMR (400 MHz, CDCl₃) δ 7.26-7.19 (m, 2H), 7.13 (dd, J = 2.2, 1.1 Hz, 1H), 6.95 (d, J = 7.7 Hz, 1H), 6.91-6.80 (m, 2H), 5.07-4.99 (m, 1H), 5.01- 4.93 (m, 1H), 3.88 (dd, J = 10.1, 5.5 Hz, 1H), 3.77 (dd, J = 10.1, 4.2 Hz, 1H), 3.72- 3.61 (m, 2H), 3.61-3.51 (m, 1H), 2.75 (s, 6H), 2.57-2.44 (m, 1H), 2.14 (s, 3H), 2.06 (d, J = 0.9 Hz, 3H), 2.04-1.94 (m, 2H), 1.68-1.44 (m, 6H). | A |
| 2as | 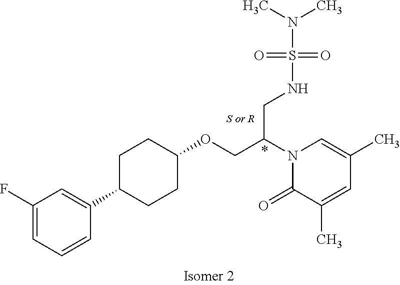 [SMILES: Cc1cc(C)c(=O)n(C(CNS(=O)(=O)N(C)C)CO[C@H]2CC[C@@H](c3cccc(F)c3)CC2)c1] | (S or R) 1-[1- [(dimethyl- sulfamoyl) amino]-3- {[(CIS)-4-(3- fluorophenyl) cyclohexyl] oxy} propan-2-yl]- 3,5-dimethyl- 1,2- dihydropyridin- 2-one- Isomer 2 | 480.2 [M + H]⁺ | ¹H NMR (400 MHz, CDCl₃) δ 7.26-7.19 (m, 2H), 7.13 (dd, J = 2.2, 1.1 Hz, 1H), 6.95 (d, J = 7.7 Hz, 1H), 6.91-6.80 (m, 2H), 5.07-4.99 (m, 1H), 5.01- 4.93 (m, 1H), 3.88 (dd, J = 10.1, 5.5 Hz, 1H), 3.77 (dd, J = 10.1, 4.2 Hz, 1H), 3.72- 3.61 (m, 2H), 3.61-3.51 (m, 1H), 2.75 (s, 6H), 2.57-2.44 (m, 1H), 2.14 (s, 3H), 2.06 (d, J = 0.9 Hz, 3H), 2.04-1.94 (m, 2H), 1.68-1.44 (m, 6H). | B |
| 2at | 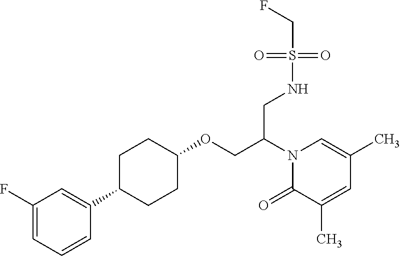 [SMILES: Cc1cc(C)c(=O)n(C(CNS(=O)(=O)CF)CO[C@H]2CC[C@@H](c3cccc(F)c3)CC2)c1] | N-[2-(3,5- dimethyl-2- oxo-1,2- dihydropyridin- 1-yl)-3- {[(CIS)-4-(3- fluorophenyl) cyclohexyl] oxy} propyl]-1- fluoromethane- sulfonamide | 469.3 [M + H]⁺ | ¹H NMR (400 MHz, CDCl₃) δ 7.30-7.20 (m, 2H), 7.15 (s, 1H), 6.94 (d, J = 7.7 Hz, 1H), 6.92-6.79 (m, 2H), 6.11 (br t, J = 5.2 Hz, 1H), 5.23-4.94 (m, 3H), 3.91 (dd, J = 10.3, 5.7 Hz, 1H), 3.85-3.69 (m, 3H), 3.65 (br s, 1H), 2.58-2.46 (m, 1H), 2.13 (s, 3H), 2.06 (s, 3H), 2.04-1.95 (m, 2H), 1.67- 1.46 (m, 6H). | A |
| 2au | 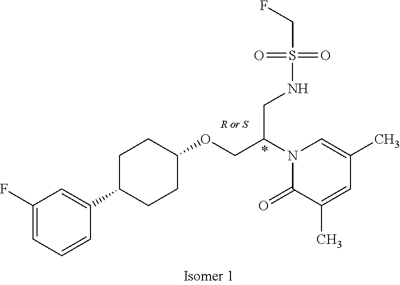 [SMILES: Cc1cc(C)c(=O)n(C(CNS(=O)(=O)CF)CO[C@H]2CC[C@@H](c3cccc(F)c3)CC2)c1] | (R or S) N-[2- (3,5-dimethyl- 2-oxo-1,2- dihydropyridin- 1-yl)-3- {[(CIS)-4-(3- fluorophenyl) cyclohexyl] oxy} propyl]-1- fluoromethane- sulfonamide- Isomer 1 | 469.3 [M + H]⁺ | ¹H NMR (500 MHz, CDCl₃) δ 7.27-7.20 (m, 2H), 7.15 (s, 1H), 6.94 (d, J = 7.7 Hz, 1H), 6.88 (td, J = 8.4, 1.9 Hz, 1H), 6.85-6.79 (m, 1H), 6.12- 6.00 (m, 1H), 5.17-4.96 (m, 3H), 3.92 (dd, J = 10.3, 5.8 Hz, 1H), 3.83-3.70 (m, 3H), 3.65 (br s, 1H), 2.58-2.47 (m, 1H), 2.13 (s, 3H), 2.06 (s, 3H), 2.04-1.94 (m, 2H), 1.67- 1.44 (m, 6H). | A |
| 2av | 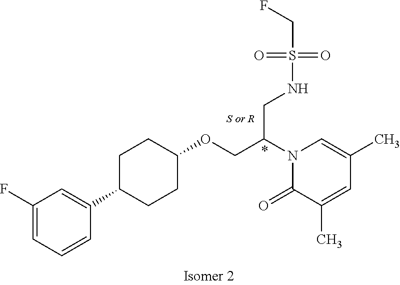 [SMILES: Cc1cc(C)c(=O)n(C(CNS(=O)(=O)CF)CO[C@H]2CC[C@@H](c3cccc(F)c3)CC2)c1] | (S or R) N-[2- (3,5-dimethyl- 2-oxo-1,2- dihydropyridin- 1-yl)-3- {[(CIS)-4-(3- fluorophenyl) cyclohexyl] oxy} propyl]-1- fluoromethane- sulfonamide- Isomer 2 | 469.3 [M + H]⁺ | ¹H NMR (400 MHz, CDCl3) δ 7.26-7.18 (m, 2H), 7.14 (dd, J = 2.5, 1.2 Hz, 1H), 6.93 (dt, J = 7.7, 1.4 Hz, 1H), 6.90-6.79 (m, 2H), 6.11 (s, 1H), 5.12 (d, J = 0.8 Hz, 1H), 5.07-4.97 (m, 2H), 3.91 (dd, J = 10.3, 5.8 Hz, 1H), 3.82-3.70 (m, 3H), 3.64 (q, J = 2.9 Hz, 1H), 2.51 (td, J = 10.3, 5.3 Hz, 1H), 2.12 (t, J = 0.8 Hz, 3H), 2.05 (d, J = 1.0 Hz, 3H), 2.04-1.94 (m, 2H), 1.66-1.45 (m, 6H). | B |
| 2ax | 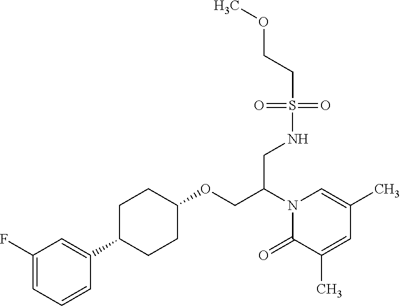 [SMILES: COCCS(=O)(=O)NCC(CO[C@H]1CC[C@@H](c2cccc(F)c2)CC1)n1cc(C)cc(C)c1=O] | N-[2-(3,5- dimethyl-2- oxo-1,2- dihydropyridin- 1-yl)-3- {[(CIS)-4-(3- fluorophenyl) cyclohexyl] oxy} propyl]-2- methoxyethane- 1-sulfonamide | 495.3 [M + H]⁺ | ¹H NMR (400 MHz, CDCl₃) δ 7.26-7.19 (m, 2H), 7.12 (s, 1H), 6.94 (d, J = 7.7 Hz, 1H), 6.91-6.78 (m, 2H), 5.20- 4.88 (m, 2H), 3.88 (dd, J = 10.1, 5.7 Hz, 1H), 3.80-3.72 (m, 3H), 3.72-3.56 (m, 3H), 3.33 (d, J = 0.7 Hz, 3H), 3.25 (t, J = 5.7 Hz, 2H), 2.61-2.40 (m, 1H), 2.13 (s, 3H), 2.05 (s, 3H), 1.99 (br t, J = 14.1 Hz, 2H), 1.68-1.44 (m, 6H). | A |
| 2ay | 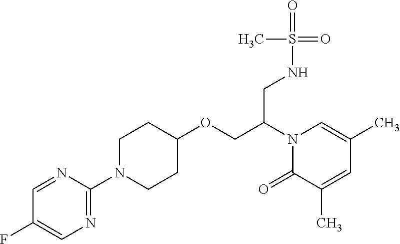 [SMILES: Cc1cc(C)c(=O)n(C(CNS(C)(=O)=O)COC2CCN(c3ncc(F)cn3)CC2)c1] | N-[2-(3,5- dimethyl-2- oxo-1,2- dihydro- pyridin- 1-yl)-3-{[1-(5- fluoro- pyrimidin- 2- yl)piperidin-4- yl]oxy}propyl] methane- sulfonamide | 454.4 [M + H]⁺ | ¹H NMR (400 MHz, CDCl₃) δ 8.18 (s, 2H), 7.16 (s, 1H), 7.11 (s, 1H), 5.09 (br t, J = 5.7 Hz, 1H), 4.98 (br dd, J = 7.5, 5.0 Hz, 1H), 4.09 (ddd, J = 9.8, 6.4, 3.1 Hz, 2H), 3.98-3.81 (m, 2H), 3.77-3.50 (m, 3H), 3.48-3.34 (m, 2H), 2.87 (s, 3H), 2.12 (s, 3H), 2.04 (s, 3H), 1.93-1.81 (m, 2H), 1.54 (br s, 2H) | D |
| 2az | 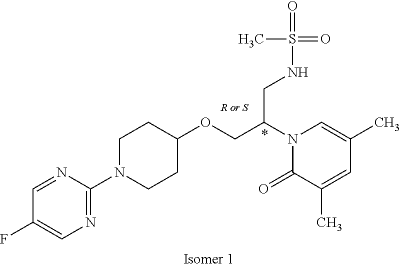 [SMILES: Cc1cc(C)c(=O)n(C(CNS(C)(=O)=O)COC2CCN(c3ncc(F)cn3)CC2)c1] | (R or S) N-[2- (3,5-dimethyl- 2-oxo-1,2- dihydro- pyridin- 1-yl)-3-{[1-(5- fluoro- pyrimidin- 2- yl)piperidin-4- yl]oxy}propyl] methane- sulfonamide- Isomer 1 | 454.2 [M + H]⁺ | ¹H NMR (400 MHz, CDCl₃) δ 8.19 (s, 2H), 7.17 (s, 1H), 7.11 (s, 1H), 5.09 (br t, J = 6.0 Hz, 1H), 5.04-4.93 (m, 1H), 4.18-4.04 (m, 2H), 3.94 (dd, J = 10.3, 5.5 Hz, 1H), 3.86 (dd, J = 10.2, 4.5 Hz, 1H), 3.77- 3.65 (m, 1H), 3.68-3.62 (m, 1H), 3.60-3.52 (m, 1H), 3.48- 3.35 (m, 2H), 2.88 (s, 3H), 2.13 (s, 3H), 2.05 (s, 3H), 1.96-1.80 (m, 2H), 1.64- 1.50 (m, 2H). | C |
| 2ba | 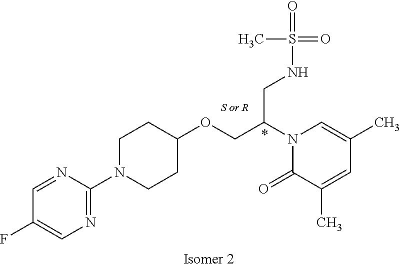 [SMILES: Cc1cc(C)c(=O)n(C(CNS(C)(=O)=O)COC2CCN(c3ncc(F)cn3)CC2)c1] | (S or R) N-[2- (3,5-dimethyl- 2-oxo-1,2- dihydro- pyridin- 1-yl)-3-{[1-(5- fluoro- pyrimidin- 2- yl)piperidin-4- yl]oxy}propyl] methane- sulfonamide- Isomer 2 | 454.2 [M + H]⁺ | ¹H NMR (400 MHz, CDCl₃) δ 8.19 (s, 2H), 7.17 (s, 1H), 7.11 (s, 1H), 5.09 (br t, J = 6.0 Hz, 1H), 5.04-4.93 (m, 1H), 4.18-4.04 (m, 2H), 3.94 (dd, J = 10.3, 5.5 Hz, 1H), 3.86 (dd, J = 10.2, 4.5 Hz, 1H), 3.77- 3.65 (m, 1H), 3.68-3.62 (m, 1H), 3.60-3.52 (m, 1H), 3.48- 3.35 (m, 2H), 2.88 (s, 3H), 2.13 (s, 3H), 2.05 (s, 3H), 1.96-1.80 (m, 2H), 1.64- 1.50 (m, 2H). | D |
| 2bb | 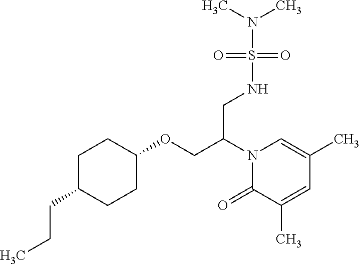 [SMILES: CCC[C@H]1CC[C@@H](OCC(CNS(=O)(=O)N(C)C)n2cc(C)cc(C)c2=O)CC1] | 1-{1- [(dimethyl- sulfamoyl) amino]- 3-{[(CIS)-4- propylcyclo- hexyl] oxy}propan- 2-yl}-3,5- dimethyl-1,2- dihydro- pyridin- 2-one | 428.5 [M + H]⁺ | ¹H NMR (400 MHz, CDCl₃) δ 7.30 (s, 1H), 7.10 (s, 1H), 5.15-5.04 (m, 1H), 4.96 (br t, J = 5.9 Hz, 1H), 3.83-3.71 (m, 2H), 3.65-3.54 (m, 2H), 3.52 (br s, 1H), 2.74 (s, 6H), 2.13 (s, 3H), 2.06 (s, 3H), 1.88- 1.76 (m, 2H), 1.53-1.34 (m, 4H), 1.34-1.22 (m, 3H), 1.21- 1.07 (m, 4H), 0.89 (t, J = 7.2 Hz, 3H). | A |
| 2bc | 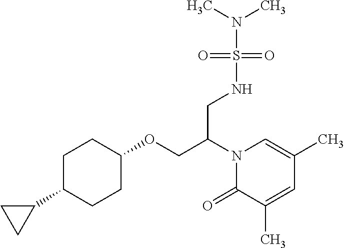 [SMILES: Cc1cc(C)c(=O)n(C(CNS(=O)(=O)N(C)C)CO[C@H]2CC[C@@H](C3CC3)CC2)c1] | 1-{1- [(dimethyl- sulfamoyl) amino]- 3-{[(CIS)-4- cyclopropyl- cyclohexyl] oxy}propan- 2-yl}-3,5- dimethyl-1,2- dihydro- pyridin- 2-one | 426.4 [M + H]⁺ | ¹H NMR (500 MHz, CDCl₃) δ 7.33-7.30 (m, 1H), 7.13-7.08 (m, 1H), 5.15-5.05 (m, 1H), 4.98 (br t, J = 6.0 Hz, 1H), 3.85- 3.73 (m, 2H), 3.64-3.52 (m, 2H), 3.52-3.47 (m, 1H), 2.74 (s, 6H), 2.14 (s, 3H), 2.07 (d, J = 0.7 Hz, 3H), 1.90-1.76 (m, 2H), 1.62-1.49 (m, 2H), 1.42- 1.33 (m, 4H), 0.59-0.46 (m, 2H), 0.43-0.34 (m, 2H), 0.04 (dd, J = 4.3, 1.4 Hz, 2H). | B |
| 2bd | 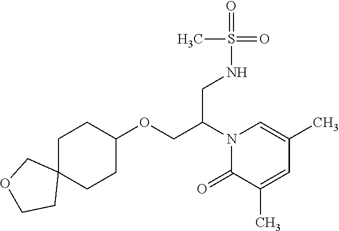 [SMILES: Cc1cc(C)c(=O)n(C(CNS(C)(=O)=O)COC2CCC3(CCOC3)CC2)c1] | N-[2-(3,5- dimethyl-2- oxo-1,2- dihydro- pyridin- 1-yl)-3-{2- oxaspiro[4.5] decan-8- yloxy}propyl] methane- sulfonamide | 413.3 [M + H]⁺ | ¹H NMR (400 MHz, CDCl₃) δ 7.24-7.17 (m, 1H), 7.11 (s, 1H), 5.17 (br t, J = 5.9 Hz, 1H), 5.08-4.94 (m, 1H), 3.92-3.75 (m, 4H), 3.73-3.57 (m, 2H), 3.55-3.46 (m, 2H), 3.39-3.26 (m, 1H), 2.87 (s, 3H), 2.13 (s, 3H), 2.09-2.04 (m, 3H), 1.85- 1.63 (m, 6H), 1.56-1.28 (m, 4H). | D |

### Example 2ak: N-[2-(2-oxo-1,2-dihydroquinolin-1-yl)-3-{[(CIS)-4-phenylcyclohexyl]oxy}propyl]methanesulfonamide

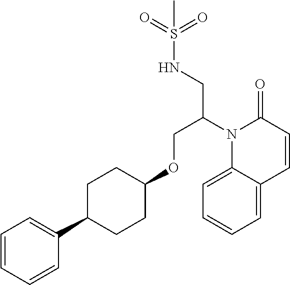 [SMILES: CS(=O)(=O)NCC(CO[C@H]1CC[C@@H](c2ccccc2)CC1)n1c(=O)ccc2ccccc21]

### 1-(1-Nitro-3-{[(CIS)-4-phenylcyclohexyl]oxy}propan-2-yl)-1,2-dihydroquinolin-2-one (INTERMEDIATE 10)

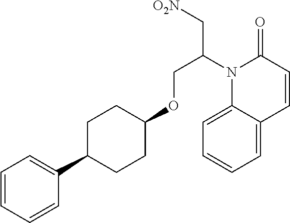 [SMILES: O=c1ccc2ccccc2n1C(CO[C@H]1CC[C@@H](c2ccccc2)CC1)C[N+](=O)[O-]]

To a suspension of 2-hydroxyquinoline (111 mg, 0.77 mmol) in THF (2 mL), tBuOK (52 mg, 0.46 mmol) and 18-Crown-6 (40 mg, 0.15 mmol) were added at 0° C. The reaction mixture was stirred at this temperature for 1 hour and then cooled to −50° C. A solution of Intermediate 7 (100 mg, 0.38 mmol) in THF (2 mL) was added and the reaction mixture was stirred at −50° C. for 15 minutes. The solution was diluted with H₂O and extracted with EtOAc. The organic layer was evaporated in vacuo. The product was purified by column chromatography using a C18 cartridge (H₂O+0.1% formic acid/MeCN+0.1% formic acid, 95:5 to 0:100) to afford the title compound (17 mg, 0.042 mmol, 11% yield). [M+H]⁺ m/z 407.3.

### 1-(1-Amino-3-{[(CIS)-4-phenylcyclohexyl]oxy}propan-2-yl)-1,2-dihydroquinolin-2-one (INTERMEDIATE 11)

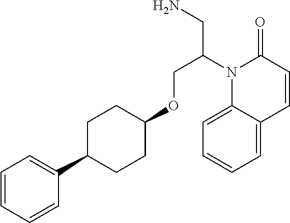 [SMILES: NCC(CO[C@H]1CC[C@@H](c2ccccc2)CC1)n1c(=O)ccc2ccccc21]

Intermediate 11 was prepared following the procedure described for Intermediate 9, starting from Intermediate 10 (17 mg, 0.042 mmol) to afford the title compound (8 mg, 0.021 mmol, 64% yield) as a white solid. [M+H]⁺ m/z 377.2.

### N-[2-(2-oxo-1,2-dihydroquinolin-1-yl)-3-{[(CIS)-4-phenylcyclohexyl]oxy}propyl]methanesulfonamide (2ak)

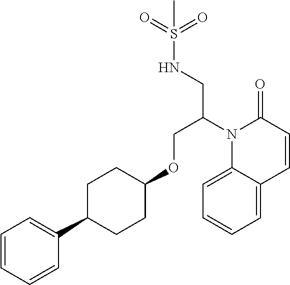 [SMILES: CS(=O)(=O)NCC(CO[C@H]1CC[C@@H](c2ccccc2)CC1)n1c(=O)ccc2ccccc21]

Example 2ak was prepared following the procedure described for Example 1, starting from Intermediate 11 (7 mg, 0.020 mmol) to afford the title compound (1.4 mg, 0.003 mmol, 16% yield) as a white solid.

TABLE 5
Characterization and EC50 hOX2 data—Compounds 2ak

| | | | Obs. | | EC50 |
|---|---|---|---|---|---|
| Ex. | Structure | Name | Mass | ¹H NMR | hOX2 |
| 2ak | 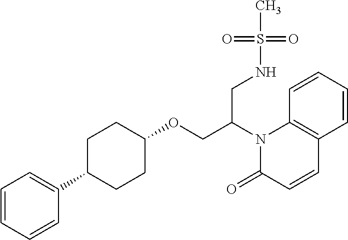 [SMILES: CS(=O)(=O)NCC(CO[C@H]1CC[C@@H](c2ccccc2)CC1)n1c(=O)ccc2ccccc21] | N-[2-(2-oxo-1,2- dihydroquinolin-1-yl)-3- {[(CIS)-4- phenylcyclohexyl]oxy} propyl] methanesulfonamide | 455.2 [M + H]⁺ | ¹H NMR (500 MHz, CDCl₃) δ 7.82-7.75 (m, 1H), 7.70 (d, J = 9.5 Hz, 1H), 7.65-7.55 (m, 2H), 7.34-7.28 (m, 1H), 7.27-7.09 (m, 3H), 6.88 (br d, J = 6.9 Hz, 2H), 6.61 (br d, J = 9.3 Hz, 1H), 5.24-5.05 (m, 2H), 4.10 (br d, J = 6.3 Hz, 2H), 4.08-3.98 (m, 1H), 3.91-3.79 (m, 1H), 3.68-3.59 (m, 1H), 2.91 (s, 3H), 2.47-2.32 (m, 1H), 2.08-1.20 (m, 8H). | D |

### Examples 3a-3d: N-[(2S,3S)-3-(3-methyl-2-oxo-1,2-dihydropyridin-1-yl)-4-{[(CIS)-4-phenylcyclohexyl]oxy}butan-2-yl]methanesulfonamide (3a); N-[(2R,3R)-3-(3-methyl-2-oxo-1,2-dihydropyridin-1-yl)-4-{[(CIS)-4-phenylcyclohexyl]oxy}butan-2-yl]methanesulfonamide (3b); N-[(2R,3S)-3-(3-methyl-2-oxo-1,2-dihydropyridin-1-yl)-4-{[(CIS)-4-phenylcyclohexyl]oxy}butan-2-yl]methanesulfonamide (3c); and N-[(2S,3R)-3-(3-methyl-2-oxo-1,2-dihydropyridin-1-yl)-4-{[(CIS)-4-phenylcyclohexyl]oxy}butan-2-yl]methanesulfonamide (3d)

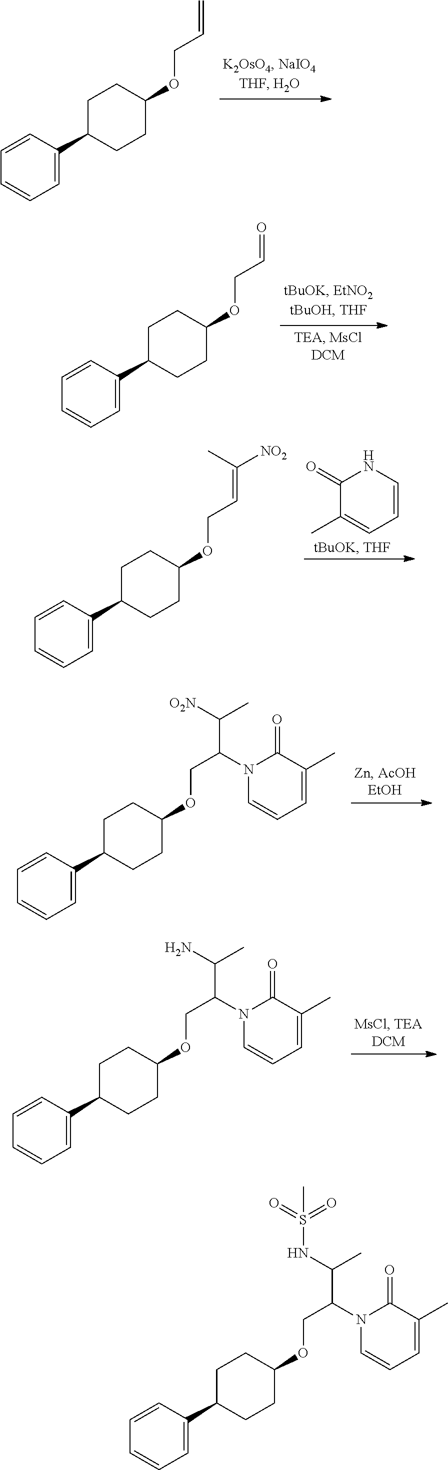

### 2-{[(CIS)-4-phenylcyclohexyl]oxy}acetaldehyde (INTERMEDIATE 12)

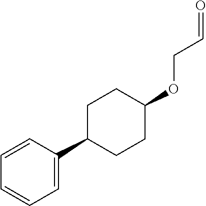 [SMILES: O=CCO[C@H]1CC[C@@H](c2ccccc2)CC1]

To a mixture of Intermediate 6 (3.24 g, 14.98 mmol) in THF (34 mL) and H₂O (68 mL) was added K₂OsO₄·2H₂O (276 mg, 0.75 mmol). The mixture was stirred at room temperature for 3 h. NaIO₄ (9.61 g, 44.93 mmol) was added and the mixture was stirred at room temperature for 3 h. The mixture was poured into H₂O, and extracted with EtOAc. The organic layer was washed with brine and concentrated in vacuo. The product was purified via column chromatography using a silica cartridge (cHex/EtOAc from 100:0 to 20:80) to afford the title compound (2.36 g, 10.81 mmol, 72% yield) as a yellow-green oil. ¹H NMR (400 MHz, CDCl₃) δ 9.81 (t, J=1.1 Hz, 1H), 7.37-7.13 (m, 5H), 4.07 (d, J=1.1 Hz, 2H), 3.71 (p, J=3.0 Hz, 1H), 2.62-2.47 (m, 2H), 2.12-2.03 (m, 2H), 1.87 (qd, J=13.0, 3.4 Hz, 2H), 1.72-1.55 (m, 3H).

### [(CIS)-4-{[-3-nitrobut-2-en-1-yl]oxy}cyclohexyl]benzene (INTERMEDIATE 13)

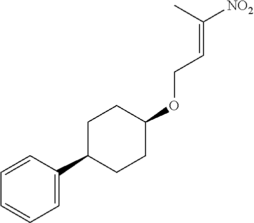 [SMILES: C/C(=C\CO[C@H]1CC[C@@H](c2ccccc2)CC1)[N+](=O)[O-]]

To a solution of Intermediate 12 (0.80 g, 3.66 mmol) in anhydrous THF (4 mL) and anhydrous tBuOH (4 mL) at 0° C. was added nitroethane (0.65 mL, 11.0 mmol) and tBuOK (41 mg, 0.37 mmol). The mixture was stirred for 2 h at room temperature, then it was diluted with Et₂O (25 mL) and washed with sat. aq. NH₄Cl (20 mL). The combined aqueous layers were extracted with Et₂O (25 mL). The organic layers were combined and concentrated in vacuo to afford the crude material (1.17 g) as a colourless oil. The crude material was dissolved in anhydrous DCM (20 mL) at 0° C., methanesulfonyl chloride (0.30 mL, 3.92 mmol) was added and stirred for 5 minutes TEA (1.09 mL, 7.84 mmol) was added at 0° C. and stirred for 15 minutes at room temperature. A saturated aqueous solution of NH₄Cl was added and the mixture was extracted with DCM (2×15 mL). The combined organic layer was concentrated in vacuo. The product was purified by column chromatography using a silica cartridge (0-10% EtOAc in cHex) to afford the title compound (594 mg, 2.16 mmol, 55% yield) as a pale-yellow oil. ¹H NMR (400 MHz, CDCl₃) δ 7.37-7.29 (m, 2H), 7.28-7.17 (m, 4H), 4.26 (dd, J=6.0, 1.2 Hz, 2H), 3.72 (t, J=3.1 Hz, 1H), 2.58 (tt, J=12.0, 3.7 Hz, 1H), 2.26 (q, J=1.2 Hz, 3H), 2.12-2.02 (m, 2H), 1.85 (qd, J=12.9, 3.4 Hz, 2H), 1.74-1.66 (m, 2H), 1.66-1.51 (m, 2H).

### 3-Methyl-1-(3-nitro-1-{[(CIS)-4-phenylcyclohexyl]oxy}butan-2-yl)-1,2-dihydropyridin-2-one (INTERMEDIATE 14)

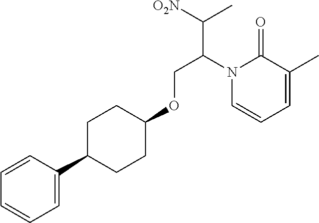 [SMILES: Cc1cccn(C(CO[C@H]2CC[C@@H](c3ccccc3)CC2)C(C)[N+](=O)[O-])c1=O]

3-Methylpyridin-2(1H)-one (106 mg, 0.97 mmol) was suspended in THF (3 mL) and tBuOK (65 mg, 0.58 mmol) and 18-Crown-6 (51 mg, 0.19 mmol) were added at 0° C. The reaction mixture was stirred for 1 h, cooled to −50° C. then Intermediate 13 (150 mg, 0.48 mmol) in THF (3 mL) was added and the reaction mixture was stirred at −50° C. for 15 minutes. The solution was diluted with H₂O and extracted with EtOAc. The aqueous layer was evaporated in vacuo to afford the crude that was purified by column chromatography using a silica cartridge (0-5% methanol in DCM) to afford the title compound (165 mg, 0.43 mmol, 89% yield). [M+H]⁺ m/z 385.2

### 1-(3-Amino-1-{[(CIS)-4-phenylcyclohexyl]oxy}butan-2-yl)-3-methyl-1,2-dihydropyridin-2-one (INTERMEDIATE 15)

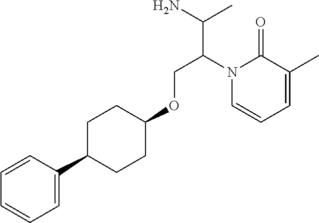 [SMILES: Cc1cccn(C(CO[C@H]2CC[C@@H](c3ccccc3)CC2)C(C)N)c1=O]

Intermediate 15 was prepared following the procedure described for Intermediate 9, starting from Intermediate 14 (164 mg, 0.43 mmol) to afford the title compound (168 mg, quant. yield) as a white solid. [M+H]⁺ m/z 355.2

### N-[(2S,3S)-3-(3-methyl-2-oxo-1,2-dihydropyridin-1-yl)-4-{[(CIS)-4-phenylcyclohexyl]oxy}butan-2-yl]methanesulfonamide (3a)

### N-[(2R,3R)-3-(3-methyl-2-oxo-1,2-dihydropyridin-1-yl)-4-{[(CIS)-4-phenylcyclohexyl]oxy}butan-2-yl]methanesulfonamide (3b)

### N-[(2R,3S)-3-(3-methyl-2-oxo-1,2-dihydropyridin-1-yl)-4-{[(CIS)-4-phenylcyclohexyl]oxy}butan-2-yl]methanesulfonamide (3c); and

### N-[(2S,3R)-3-(3-methyl-2-oxo-1,2-dihydropyridin-1-yl)-4-{[(CIS)-4-phenylcyclohexyl]oxy}butan-2-yl]methanesulfonamide (3d)

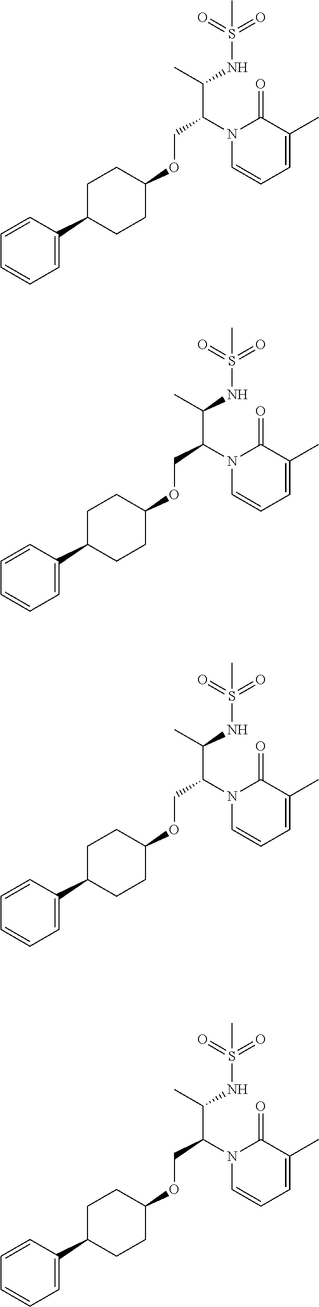 [SMILES: Cc1cccn([C@H](CO[C@H]2CC[C@@H](c3ccccc3)CC2)[C@H](C)NS(C)(=O)=O)c1=O ; Cc1cccn([C@@H](CO[C@H]2CC[C@@H](c3ccccc3)CC2)[C@@H](C)NS(C)(=O)=O)c1=O ; Cc1cccn([C@H](CO[C@H]2CC[C@@H](c3ccccc3)CC2)[C@@H](C)NS(C)(=O)=O)c1=O ; Cc1cccn([C@@H](CO[C@H]2CC[C@@H](c3ccccc3)CC2)[C@H](C)NS(C)(=O)=O)c1=O]

Examples 3a, 3b, 3c, & 3d were prepared following the procedure described for Example 1, starting from Intermediate 15 (145 mg, 0.31 mmol), followed by preparative HPLC (MDAP Waters, column: xBridge C18 (30×100 mm, 3 μm). Conditions: [A: 10 mM aq. NH₄HCO₃ adjusted to pH 10 with NH₃]; [B: MeCN]. Gradient: from 49.0% B to 51.0% B in 10 min (flow: 40.00 mL/min) to afford fraction A (15 mg) and fraction B (15 mg). Isomer 1 was assigned to the first eluting compound and Isomer 2 to the second eluting compound during chiral separation using one of the conditions reported above.

Fraction A was submitted to chiral separation (Chiralpak AD-H, 25×2.0 cm, 5 m, 70/30 n-Hexane: (Ethanol/Methanol 1:1+0.1% IPA, floe: 17 m-min)) to afford Example 41, 5.8 mg, 0.013 mmol, 4% yield, 100% ee, r.t. 7.0 min) and Example 42 (6.2 mg, 0.014 mmol, 5% yield, 100% ee, r.t. 10.9 min).

Fraction B was submitted to chiral separation (Chiralpak AD-H, 25×2.0 cm, 5 (m, 55/45 n-Hexane: (Ethanol/Methanol 1:1+0.1% IPA, floe: 17 mL/min)) to afford Example 43 (4.6 mg, 0.011 mmol, 3% yield, 100% ee, r.t. 6.4 min) and Example 44 (4.6 mg, 0.011 mmol, 3% yield, 100% ee, 10.5 min) as white solids.

TABLE 6
Characterization and EC50 hOX2 data-Compounds 3a-3d

| | | | Obs. | | EC50 |
|---|---|---|---|---|---|
| Ex. | Structure | Name | Mass | ¹H NMR | hOX2 |
| 3a | 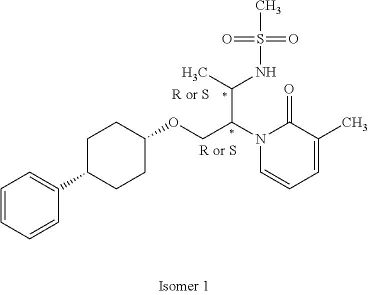 [SMILES: Cc1cccn(C(CO[C@H]2CC[C@@H](c3ccccc3)CC2)C(C)NS(C)(=O)=O)c1=O] | (2R or 2S) and (3R or 3S) N- [3-(3-methyl-2-oxo-1,2- dihydropyridin-1-yl)-4- {[(CIS)-4- phenylcyclohexyl]oxy} butan- 2-yl]methanesulfonamide— Isomer 1 | 433.2 [M + H]⁺ | ¹H NMR (500 MHz, CDCl₃) δ 7.75 (br d, J = 6.6 Hz, 1H), 7.35-7.30 (m, 2H), 7.27-7.18 (m, 4H), 6.18 (t, J = 6.9 Hz, 1H), 5.57 (d, J = 9.6 Hz, 1H), 5.07 (br s, 1H), 4.15-4.04 (m, 2H), 3.85 (dd, J = 10.8, 2.5 Hz, 1H), 3.71 (t, J = 2.6 Hz, 1H), 2.75 (s, 3H), 2.63-2.54 (m, 1H), 2.17 (s, 3H), 2.13-2.02 (m, 2H), 1.82-1.59 (m, 6H), 1.37 (d, J = 6.7 Hz, 3H). | C |
| 3b | 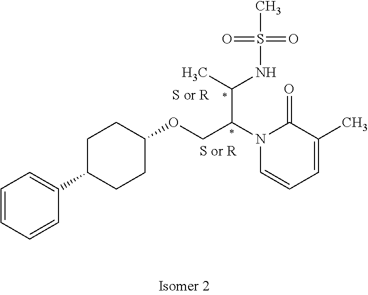 [SMILES: Cc1cccn(C(CO[C@H]2CC[C@@H](c3ccccc3)CC2)C(C)NS(C)(=O)=O)c1=O] | (2S or 2R) and (3S or 3R) N- [3-(3-methyl-2-oxo-1,2- dihydropyridin-1-yl)-4- {[(CIS)-4- phenylcyclohexyl]oxy}butan- 2-yl]methanesulfonamide— Isomer 2 | 433.2 [M + H]⁺ | ¹H NMR (500 MHz, CDCl₃) δ 7.75 (br d, J = 6.6 Hz, 1H), 7.30-7.35 (m, 2H), 7.18-7.27 (m, 4H), 6.18 (t, J = 6.9 Hz, 1H), 5.57 (d, J = 9.6 Hz, 1H), 5.07 (br s, 1H), 4.04-4.15 (m, 2H), 3.85 (dd, J = 10.8, 2.5 Hz, 1H), 3.71 (t, J = 2.6 Hz, 1H), 2.75 (s, 3H), 2.54-2.63 (m, 1H), 2.17 (s, 3H), 2.02-2.13 (m, 2H), 1.59-1.82 (m, 6H), 1.37 (d, J = 6.7 Hz, 3H). | C |
| 3c | 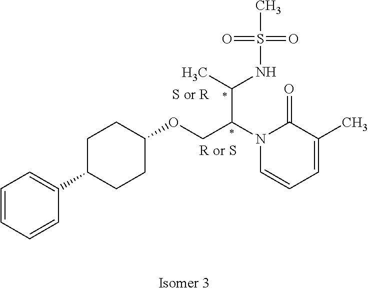 [SMILES: Cc1cccn(C(CO[C@H]2CC[C@@H](c3ccccc3)CC2)C(C)NS(C)(=O)=O)c1=O] | (2S or 2R) and (3R or 3S) N- [3-(3-methyl-2-oxo-1,2- dihydropyridin-1-yl)-4- {[(CIS)-4- phenylcyclohexyl]oxy}butan- 2-yl]methanesulfonamide— Isomer 3 | 433.2 [M + H]⁺ | ¹H NMR (400 MHz, CDCl₃) δ 7.77 (br s. 1H), 7.29-7.35 (m, 3H), 7.11-7.26 (m, 4H), 6.21 (t, J = 6,8 Hz, 1H), 4.61 (s, 1H), 4.05-4.20 (m, 1H), 3.83-4.04 (m, 1H), 3.77 (dd, J = 10.5, 2.2 Hz, 1H), 3.60-3.67 (m, 1H), 2.58-2.83 (m, 3H), 2.46-2.58 (m, 1H), 2.16 (2, 3H), 1.94-2.09 (m, 2H), 1.48-1.76 (m, 6H), 1.38-1.47 (m, 3H). | D |
| 3d | 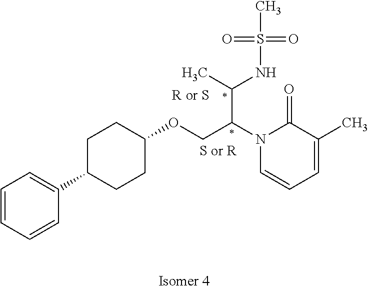 [SMILES: Cc1cccn(C(CO[C@H]2CC[C@@H](c3ccccc3)CC2)C(C)NS(C)(=O)=O)c1=O] | (2R or 2S) and (3S or 3R) N- [3-(3-methyl-2-oxo-1,2- dihydropyridin-1-yl)-4- {[(CIS)-4- phenylcyclohexyl]oxy}butan- 2-yl]methanesulfonamide— Isomer 4 | 433.2 [M + H]⁺ | ¹H NMR (400 MHz, CDCl₃) δ 7.77 (br s, 1H), 7.35-7.29 (m, 3H), 7.26-7.11 (m, 4H), 6.21 (t, J = 6.8 Hz, 1H), 4.61 (s, 1H), 4.20-4.05 (m, 1H), 4.04-3.83 (m, 1H), 3.77 (dd, J = 10.5, 2.2 Hz, 1H), 3.67-3.60 (m, 1H), 2.83-2.58 (m, 3H), 2.58-2.46 (m, 1H), 2.16 (s, 3H), 2.09-1.94 (m, 2H), 1.76-1.48 (m, 6H), 1.47-1.38 (m, 3H). | C |

### Example 4: N-[2-(1-methyl-2-oxo-1,2-dihydropyridin-3-yl)-3-{[(CIS)-4-phenylcyclohexyl]oxy}propyl]methanesulfonamide (4)

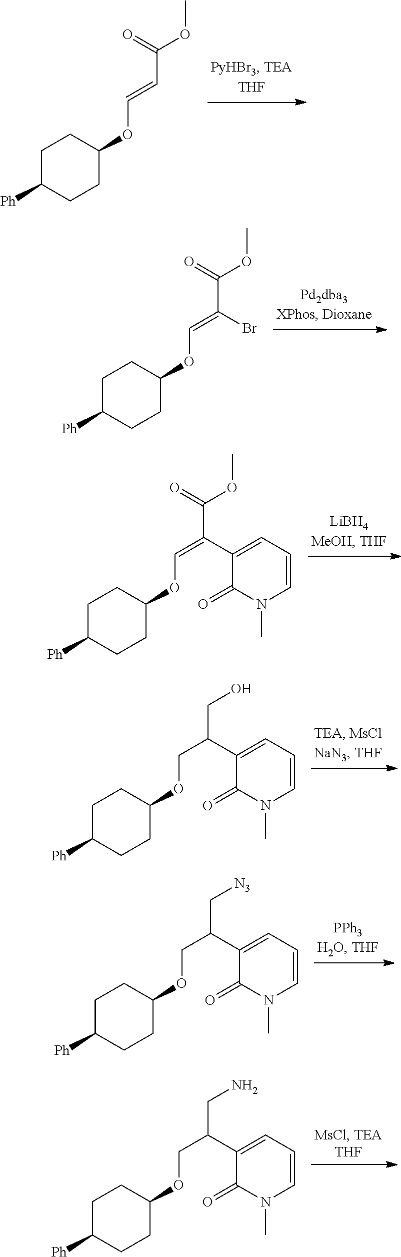 [SMILES: COC(=O)/C=C/O[C@H]1CC[C@@H](c2ccccc2)CC1 ; COC(=O)/C(Br)=C/O[C@H]1CC[C@@H](c2ccccc2)CC1 ; COC(=O)/C(=C/O[C@H]1CC[C@@H](c2ccccc2)CC1)c1cccn(C)c1=O ; Cn1cccc(C(CO)CO[C@H]2CC[C@@H](c3ccccc3)CC2)c1=O ; Cn1cccc(C(CN=[N+]=[N-])CO[C@H]2CC[C@@H](c3ccccc3)CC2)c1=O ; Cn1cccc(C(CN)CO[C@H]2CC[C@@H](c3ccccc3)CC2)c1=O]

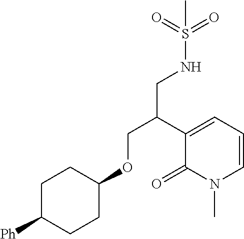 [SMILES: Cn1cccc(C(CNS(C)(=O)=O)CO[C@H]2CC[C@@H](c3ccccc3)CC2)c1=O]

### Methyl-2-bromo-3-{[(CIS)-4-phenylcyclohexyl]oxy}prop-2-enoate (INTERMEDIATE 16)

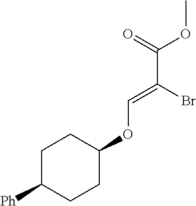 [SMILES: COC(=O)/C(Br)=C/O[C@H]1CC[C@@H](c2ccccc2)CC1]

To a solution of pyridinium tribromide (4.40 g, 13.7 mmol) in anhydrous-THF (30 mL) was added Intermediate 1 (3.41 g, 13.1 mmol) in anhydrous-THF (2×10 mL) at 0° C. The reaction was warmed to room temperature and stirred for 0.5 h. The solution was cooled to 0° C. then TEA (3.6 mL, 26.2 mmol) was added. The reaction mixture was warmed to room temperature for 0.5 h. The reaction was quenched with H₂O (20 mL) and extracted with DCM (3×75 mL). The combined organic layers were dried over MgSO₄, filtered and concentrated in vacuo. The product was purified by column chromatography using a silica cartridge (0-10% EtOAc in heptane) to afford the title compound (3.50 g, 10.32 mmol, 79% yield) as an off-white solid. [M+H]⁺ m/z 339.2 & 341.2

### Methyl-2-(1-methyl-2-oxo-1,2-dihydropyridin-3-yl)-3-{[(CIS)-4-phenylcyclohexyl]oxy}prop-2-enoate (INTERMEDIATE 17)

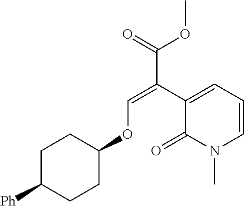 [SMILES: COC(=O)/C(=C/O[C@H]1CC[C@@H](c2ccccc2)CC1)c1cccn(C)c1=O]

A solution of Intermediate 16 (75 mg, 0.22 mmol) and (2-methoxy-3-pyridyl)boronic acid (51 mg, 0.33 mmol) in aqueous 2M Na₂CO₃ (0.33 mL, 0.66 mmol) and 1,4-dioxane (0.4 mL) was degassed for 5 min with N₂ then Pd₂(dba)₃ (51 mg, 0.055 mmol) and XPhos (53 mg, 0.11 mmol) were added. The reaction mixture was heated at 100° C. for 1 h. The reaction was filtered through Celite and washed with EtOAc. The combined organic layers were concentrated in vacuo and purified by column chromatography using a silica cartridge (0-20% EtOAc in heptane) to afford the title compound (70 mg, 0.19 mmol, 87% yield) as a dark red gum. [M+H]⁺ m/z 368.3

### 3-(1-Hydroxy-3-{[(CIS)-4-phenylcyclohexyl]oxy}propan-2-yl)-1-methyl-1,2-dihydropyridin-2-one (INTERMEDIATE 18)

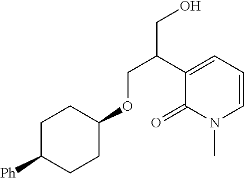 [SMILES: Cn1cccc(C(CO)CO[C@H]2CC[C@@H](c3ccccc3)CC2)c1=O]

Intermediate 18 was prepared following the procedure described for Intermediate 3, starting from Intermediate 17 (200 mg, 0.54 mmol) to afford the title compound (232 mg, quant. yield). [M+H]⁺ m/z 342.4

### 3-(1-Hydroxy-3-{[(CIS)-4-phenylcyclohexyl]oxy}propan-2-yl)-1-methyl-1,2-dihydropyridin-2-one (INTERMEDIATE 19)

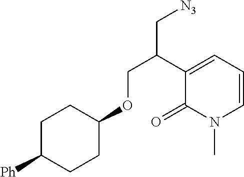 [SMILES: Cn1cccc(C(CN=[N+]=[N-])CO[C@H]2CC[C@@H](c3ccccc3)CC2)c1=O]

Intermediate 19 was prepared following the procedure described for Intermediate 4, starting from Intermediate 18 (388 mg, 1.14 mmol) to afford the title compound (410 mg, quant. yield). [M+H]⁺ m/z 367.4

### 3-(1-Hydroxy-3-{[(CIS)-4-phenylcyclohexyl]oxy}propan-2-yl)-1-methyl-1,2-dihydropyridin-2-one (INTERMEDIATE 20)

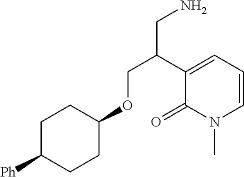 [SMILES: Cn1cccc(C(CN)CO[C@H]2CC[C@@H](c3ccccc3)CC2)c1=O]

Intermediate 20 was prepared following the procedure described for Intermediate 5, starting from Intermediate 19 (410 mg, 1.12 mmol) to afford the title compound (94 mg, 0.28 mmol, 25% yield). [M+H]⁺ m/z 341.4

### N-[2-(1-methyl-2-oxo-1,2-dihydropyridin-3-yl)-3-{[(CIS)-4-phenylcyclohexyl]oxy}propyl]methanesulfonamide (4)

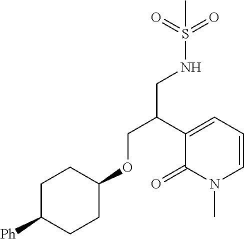 [SMILES: Cn1cccc(C(CNS(C)(=O)=O)CO[C@H]2CC[C@@H](c3ccccc3)CC2)c1=O]

Example 4 was prepared following the procedure described for Example 1, starting from Intermediate 20 (71 mg, 0.21 mmol) to afford the title compound (35 mg, 0.082 mmol, 40% yield) as a white solid.

Enantiomers were separated by chiral purification using Chiralpak columns (AD-H, IC, OJ-H), and eluting with a mixture of n-Hexane/EtOH, n-Hexane/(EtOH+0.1% iPrNH₂), n-Hexane/(EtOH/MeOH 1:1+0.1% iPrNH₂), with ratios between 35:65 and 70:30. Isomer 1 was assigned to the first eluting compound and Isomer 2 to the second eluting compound during chiral separation using one of the conditions reported above.

TABLE 7
Characterization and EC50 hOX2 data—Compounds 4-4b

| | | | Obs. | | EC50 |
|---|---|---|---|---|---|
| Ex. | Structure | Name | Mass | ¹H NMR | hOX2 |
| 4 | 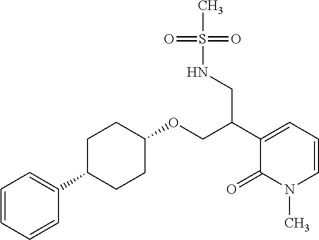 [SMILES: Cn1cccc(C(CNS(C)(=O)=O)CO[C@H]2CC[C@@H](c3ccccc3)CC2)c1=O] | N-[2-(1-methyl-2-oxo-1,2- dihydropyridin-3-yl)-3- {[(CIS)-4- phenylcyclohexyl]oxy}propyl] methanesulfonamide | 419.6 [M + H]⁺ | ¹H NMR (400 MHz, CDCl₃) δ 7.39 (dd, J = 6.9, 1.9 Hz, 1H), 7.32-7.27 (m, 2H), 7.22-7.15 (m, 3H), 6.20 (t, J = 6.8 Hz, 1H), 3.76-3.69 (m, 2H), 3.65 (q, J = 2.8 Hz, 1H), 3.56 (s, 3H), 3.56-3.43 (m, 3H), 2.94 (s, 3H), 2.52 (tt, J = 11.6, 3.7 Hz, 1H), 2.07-1.96 (m, 2H), 1.80-1.61 (m, 5H), 1.58-1.46 (m, 2H). | C |
| 4a | 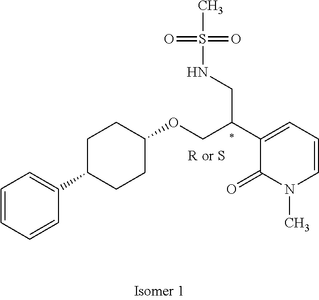 [SMILES: Cn1cccc(C(CNS(C)(=O)=O)CO[C@H]2CC[C@@H](c3ccccc3)CC2)c1=O] | (R or S) N-[2-(1-methyl-2- oxo-1,2-dihydropyridin-3-yl)- 3-{[(CIS)-4- phenylcyclohexyl]oxy}propyl] methanesulfonamide—Isomer 1 | 419.2 M + H]⁺ | ¹H NMR (400 MHz, CDCl₃) δ 7.38 (dd, J = 6.9, 2.0 Hz, 1H), 7.26 (s, 3H), 7.23-7.15 (m, 3H), 6.18 (t, J = 6.8 Hz, 1H), 5.48 (d, J = 6.0 Hz, 1H), 3.77-3.70 (m, 2H), 3.65 (t, J = 3.0 Hz, 1H), 3.61-3.43 (m, 6H), 2.94 (s, 3H), 2.52 (tt, J = 11.8, 3.8 Hz, 1H), 2.03 (dd, J = 13.9, 3.2 Hz, 2H), 1.77-1.60 (m, 4H), 1.54 (s, 2H). | C |
| 4b | 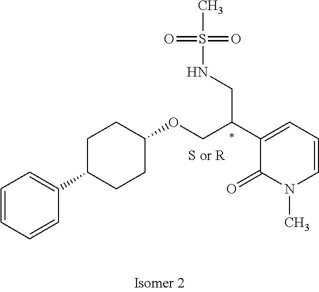 [SMILES: Cn1cccc(C(CNS(C)(=O)=O)CO[C@H]2CC[C@@H](c3ccccc3)CC2)c1=O] | (S or R) N-[2-(1-methyl-2- oxo-1,2-dihydropyridin-3-yl)- 3-{[(CIS)-4- phenylcyclohexyl]oxy}propyl] methanesulfonamide—Isomer 2 | 419.2 [M + H]⁺ | ¹H NMR (400 MHz, CDCl₃) δ 7.38 (dd, J = 7.0, 2.0 Hz, 1H), 7.32-7.23 (m, 3H), 7.23-7.14 (m, 3H), 6.18 (t, J = 6.8 Hz, 1H), 5.48 (d, J = 6.0 Hz, 1H), 3.79-3.68 (m, 2H), 3.64 (d, J = 3.1 Hz, 1H), 3.61-3.40 (m, 6H), 2.94 (s, 3H), 2.52 (tt, J = 11.8, 3.8 Hz, 1H), 2.03 (dd, J = 14.1, 3.2 Hz, 2H), 1.80-1.60 (m, 4H), 1.54 (s, 2H). | D |

### Example 5a: N-[2-(1,5-dimethyl-2-oxo-1,2-dihydropyridin-3-yl)-3-{[(CIS)-4-phenylcyclohexyl]oxy}propyl] methanesulfonamide (5a)

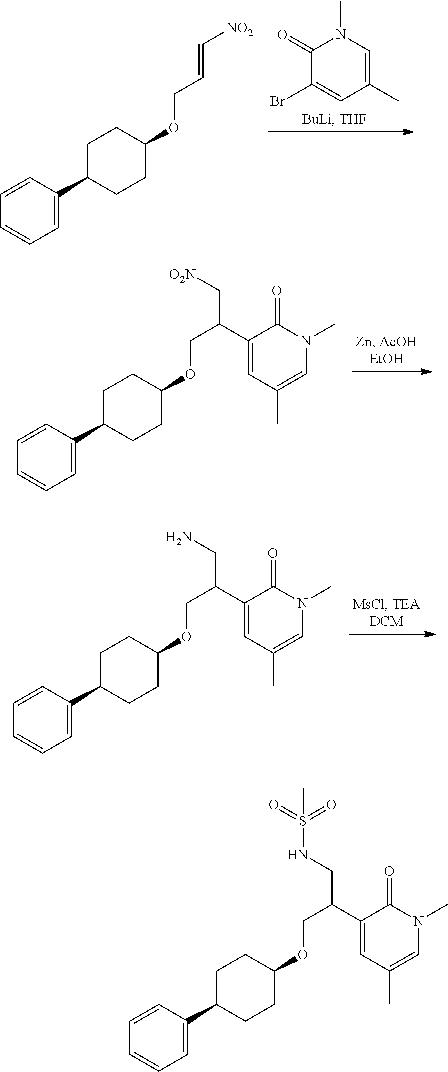

### 1,5-Dimethyl-3-(1-nitro-3-{[(CIS)-4-phenylcyclohexyl]oxy}propan-2-yl)-1,2-dihydropyridin-2-one (INTERMEDIATE 21)

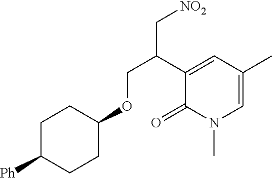 [SMILES: Cc1cc(C(CO[C@H]2CC[C@@H](c3ccccc3)CC2)C[N+](=O)[O-])c(=O)n(C)c1]

To a solution of 3-bromo-1,5-dimethylpyridin-2-one (93 mg, 0.46 mmol) in anhydrous THF (2 mL) at −78° C., a 2.5 M solution of nBuLi in THF (0.18 mL, 0.46 mmol) was added dropwise. The solution was stirred at −78° C. for 45 minutes. A solution of Intermediate 7 (100 mg, 0.38 mmol) in THF (2 mL) was added dropwise and the solution was stirred at −78° C. for 30 minutes. The reaction was quenched by addition of a solution of saturated aqueous NH₄Cl at −78° C. and allowed to reach room temperature. The solution was diluted with EtOAc and washed with H₂O. The organic layer was evaporated in vacuo and the product was purified by column chromatography using a silica cartridge (0-100% EtOAc in cHex) to afford the title compound (58 mg, 0.15 mmol, 39% yield) as a pale yellow oil. [M+H]+ m/z 385.3.

### INTERMEDIATE 21a

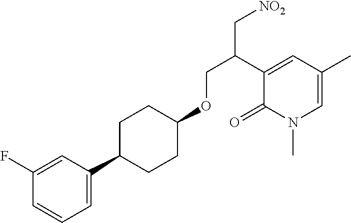 [SMILES: Cc1cc(C(CO[C@H]2CC[C@@H](c3cccc(F)c3)CC2)C[N+](=O)[O-])c(=O)n(C)c1]

### 3-[1-[[4-(3-fluorophenyl)cyclohexoxy]methyl]-2-nitro-ethyl]-1,5-dimethyl-pyridin-2-one

To a solution of 3-bromo-1,5-dimethyl-1,2-dihydropyridin-2-one (145 mg, 0.72 mmol) in THF (2.1 mL) under N₂ atmosphere, a 1.3 M solution of isopropylmagnesium chloride lithium chloride complex in THF (0.55 mL, 0.72 mmol) was added at RT. The mixture was stirred for 10 min, then CuI (150 mg, 0.79 mmol) was added and the reaction mixture was stirred for 1 h. After consumption of starting material, a solution of 1-fluoro-3-[4-[(E)-3-nitroallyloxy]cyclohexyl]benzene (200 mg, 0.72 mmol) in THF (0.7 mL) was added and the reaction mixture was stirred for 2 h at RT. The mixture was quenched at RT by addition of a solution of saturated aqueous NH₄Cl. The solution was diluted with EtOAc and washed with H₂O. The organic layer was evaporated in vacuo to afford the title compound (247 mg, 0.61 mmol, 86% yield) as a yellow oil. [M+H]⁺ m/z 403.9

### INTERMEDIATE 21b

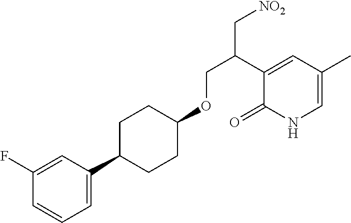 [SMILES: Cc1c[nH]c(=O)c(C(CO[C@H]2CC[C@@H](c3cccc(F)c3)CC2)C[N+](=O)[O-])c1]

### 5-methyl-3-(1-nitro-3-{[(CIS)-4-(3-fluorophenyl)cyclohexyl]oxy}propan-2-yl)-1,2-dihydropyridin-2-one

To a solution of 2-methoxy-5-methyl-3-(1-nitro-3-{[(CIS)-4-(3-fluorophenyl)cyclohexyl]oxy}propan-2-yl)pyridine (283 mg, 0.41 mmol) (prepared following the same procedure described for the synthesis of Intermediate 21 starting from 3-bromo-2-methoxy-5-methylpyridine) in acetic acid (2 mL), KI (406 mg, 2.45 mmol) was added. The emulsion was stirred at 90° C. for 18h, then the mixture was poured into a sat. aq. NaHCO₃ solution. EtOAc was added and the mixture washed once more with NaHCO₃. The organic phase was dried (Na₂SO₄) and evaporated in vacuo. The product was purified by column chromatography using a silica cartridge 12g (DCM/MeOH from 100:0 to 95:5) to afford the title compound (163 mg, quant. yield) as a red-brown oil. [M+H]⁺ m/z 389.4

### INTERMEDIATE 21c

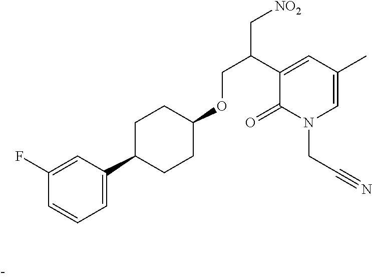 [SMILES: Cc1cc(C(CO[C@H]2CC[C@@H](c3cccc(F)c3)CC2)C[N+](=O)[O-])c(=O)n(CC#N)c1]

### 2-[5-methyl-3-(1-nitro-3-{[(CIS)-4-(3-fluorophenyl)cyclohexyl]oxy}propan-2-yl)-2-oxo-1,2-dihydropyridin-1-yl]acetonitrile

To a stirred suspension of Intermediate 21b (50 mg, 0.13 mmol) and K₂CO₃ (0.04 g, 0.26 mmol) in DMF (1 mL), 2-bromoacetonitrile (0.02 mL, 0.26 mmol) was added and the mixture was stirred at RT for 2 h. The mixture was diluted with EtOAc and washed with water. The organic layer was dried through a hydrophobic funnel and evaporated in vacuo to afford the title compound (65 mg, quant. yield) as a brown oil. [M+H]⁺ m/z 428.3

### INTERMEDIATE 21d

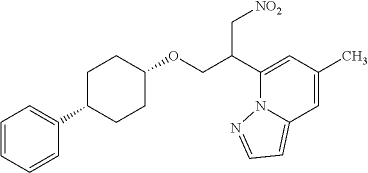 [SMILES: Cc1cc(C(CO[C@H]2CC[C@@H](c3ccccc3)CC2)C[N+](=O)[O-])n2nccc2c1]

### 2-[5-methyl-3-(1-nitro-3-{[(CIS)-4-(3-fluorophenyl)cyclohexyl]oxy}propan-2-yl)-2-oxo-1,2-dihydropyridin-1-yl]acetonitrile

To a solution of 5-methylpyrazolo[1,5-a]pyridine (104 mg, 0.79 mmol) under N₂ atmosphere at −78° C., a 2.5M solution of nBuLi (0.34 mL, 0.86 mmol) in hexane was added. The mixture was stirred for 30 minutes, then Intermediate 7 (200 mg, 0.72 mmol) was added and the reaction mixture was stirred at −78° C. for 1 h. The mixture was quenched by addition of sat. aq. NH₄Cl and washed extracted with EtOAc. The organic layer was dried through a hydrophobic funnel and evaporated in vacuo. The product was purified by column chromatography using a silica cartridge (cHex/EtOAc from 100:0 to 70:30) to afford the title compound (231 mg, 0.56 mmol, 78% yield) as an orange oil. [M+H]⁺ m/z 412.3

### INTERMEDIATE 21e

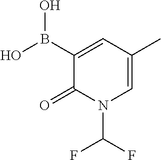

### 1-(difluoromethyl)-5-methyl-2-oxo-1,2-dihydropyridin-3-yl]boronic acid

A mixture of KOAc (5.77 g, 58.82 mmol), [Pd(dppf)Cl₂] (1.2 g, 1.47 mmol), 3-bromo-1-(difluoromethyl)-5-methyl-pyridin-2-one (7.0 g, 29.41 mmol), and B₂Pin₂ (7.47 g, 29.41 mmol) in anhydrous CPME (100 mL) was degassed through three N₂-vacuum cycles. The mixture was then stirred at 90° C. for 16 h. The reaction was cooled down, filtered through a pad of Celite. The solution was concentrated in vacuo and the product was purified by column chromatography using a C18 cartridge (H₂O+FA 0.1%/MeCN+FA 0.1%, from 95:5 to 65:35) to afford the title compound (3 g, 14.78 mmol, 50% yield) as a white solid. [M+H]⁺ m/z 204.1

### INTERMEDIATE 21f

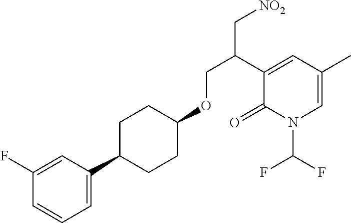 [SMILES: Cc1cc(C(CO[C@H]2CC[C@@H](c3cccc(F)c3)CC2)C[N+](=O)[O-])c(=O)n(C(F)F)c1]

### 1-(difluoromethyl)-5-methyl-2-oxo-3-pyridyl]boronic acid

Under N₂ atmosphere, chlorobis(ethylene)rhodium dimer (7 mg, 0.02 mmol) and (rac)-BINAP (23 mg, 0.04 mmol) in toluene (2 mL) were stirred at RT for 1 h. A solution of Intermediate 21e (109 mg, 0.54 mmol) and 1-fluoro-3-[4-[(E)-3-nitroallyloxy]cyclohexyl]benzene (50 mg, 0.18 mmol) in toluene (0.5 mL) was added, followed by a 0.75 M solution of potassium hydroxide in water (0.12 mL, 0.09 mmol). The reaction mixture was stirred at 100° C. for 1 h. The mixture was filtered over a pad of Celite and washed with EtOAc. The organic layer was washed with a solution of saturated aqueous NaHCO₃, then evaporated in vacuo. The product was purified by column chromatography using a silica cartridge (0-20% EtOAc in cHex) to afford the title compound (20 mg, 0.05 mmol, 26% yield) as an orange oil. [M+H]⁺ m/z 439.3

### 3-(1-Amino-3-{[(CIS)-4-phenylcyclohexyl]oxy}propan-2-yl)-1,5-dimethyl-1,2-dihydropyridin-2-one (INTERMEDIATE 22)

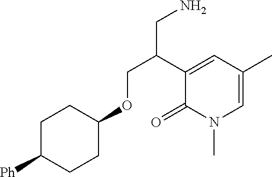 [SMILES: Cc1cc(C(CN)CO[C@H]2CC[C@@H](c3ccccc3)CC2)c(=O)n(C)c1]

Intermediate 22 was prepared following the procedure described for Intermediate 9, starting from Intermediate 21 (58 mg, 0.15 mmol) to afford the title compound (39 mg, 0.11 mmol, 74% yield). [M+H]⁺ m/z 355.2

### N-[2-(1,5-dimethyl-2-oxo-1,2-dihydropyridin-3-yl)-3-{[(CIS)-4-phenylcyclohexyl]oxy}propyl] methanesulfonamide (5a)

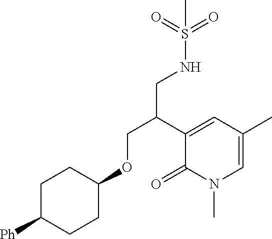 [SMILES: Cc1cc(C(CNS(C)(=O)=O)CO[C@H]2CC[C@@H](c3ccccc3)CC2)c(=O)n(C)c1]

Example 5a was prepared following the procedure described for Example 1, starting from Intermediate 22 (39 mg, 0.11 mmol) to afford the title compound (9 mg, 0.022 mmol, 19% yield).

### 2-(1-methanesulfonamido-3-{[(CIS)-4-phenylcyclohexyl]oxy}propan-2-yl)-4,6-dimethylpyridin-1-ium-1-olate (5j)

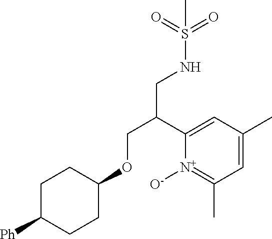 [SMILES: Cc1cc(C)[n+]([O-])c(C(CNS(C)(=O)=O)CO[C@H]2CC[C@@H](c3ccccc3)CC2)c1]

To a mixture of Example 5i (15 mg, 0.04 mmol) in DCM (1 mL) was added 3-chlorobenzenecarboperoxoic acid (13.32 mg, 0.05 mmol) and the mixture was stirred at RT for 3 h. Solid K₂CO₃ was added, and the mixture was stirred for 10 min at RT. The mixture was diluted with DCM and washed with H₂O. The organic layer was evaporated in vacuo and the product was purified by column chromatography using a silica cartridge (0-5% methanol in DCM) to afford the title compound (8 mg, 0.02 mmol, 53% yield) as a white solid.

The following examples were prepared following the procedure described for Example 5a using the appropriate heterocyclic reagents. The starting materials are either prepared as described in the intermediates section, commercially available, or may be prepared from commercially available reagents using conventional reactions well known in the art. Examples 5c and 5d were prepared using F-phenylcyclohexanol instead of phenylcyclohexanol following the same 3 step procedure described for the synthesis of Example 5a. Example 5e was prepared following the same procedure described for the synthesis of Example 5a starting from Intermediate 21c. Example 5g was prepared following the same procedure described for the synthesis of Example 5a starting from Intermediate 21d. Examples 5m-5w were prepared following the same 3-steps procedure described for the synthesis of Example 5a with the only variation of the first step, which was performed following the procedure used for Intermediate 21a, reacting the appropriate nitroalkene derivative with the appropriate heterocyclic reagent. Examples 5x-5ac were prepared following the same 3-steps procedure described for the synthesis of Example 5a, with the only variation of the first step, which was performed following the procedure used for Intermediates 21f, reacting the appropriate nitroalkene derivative with Intermediate 21e. F-analogues were prepared using F-phenyl cyclohexanol instead of phenyl cyclohexanol following the same procedure described for the synthesis of Example 5a. Sulfonamide analog was obtained reacting the amine with the appropriate sulfonyl chloride following the procedure used for the synthesis of Example 5a. Enantiomers were separated by chiral purification using Chiralpak columns (AD-H, AS-H, IC, OJ-H), and eluting with a mixture of n-Hexane/EtOH, n-Hexane/(EtOH+0.1% iPrNH₂), n-Hexane/(EtOH/MeOH 1:1+0.1% iPrNH₂), with ratios between 35:65 and 70:30. Isomer 1 was assigned to the first eluting compound and Isomer 2 to the second eluting compound during chiral separation using one of the conditions reported above. The products are reported in the table below.

TABLE 8
Characterization and EC50 hOX2 data—Compounds 5a-5d

| | | | Obs. | | EC50 |
|---|---|---|---|---|---|
| Ex. | Structure | Name | Mass | ¹H NMR | hOX2 |
| 5a | 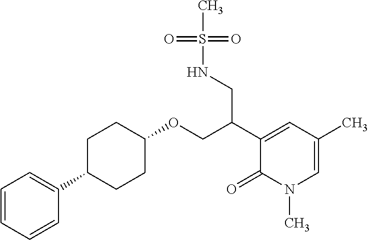 [SMILES: Cc1cc(C(CNS(C)(=O)=O)CO[C@H]2CC[C@@H](c3ccccc3)CC2)c(=O)n(C)c1] | N-[2-(1,5-dimethyl-2- oxo-1,2- dihydropyridin-3-yl)-3- {[(CIS)-4- phenylcyclohexyl]oxy} propyl] methanesulfonamide | 433.3 [M + H]⁺ | ¹H NMR (500 MHz, CDCl₃) δ 7.32-7.28 (m, 2H), 7.24 (d, J = 2.5 Hz, 1H), 7.23-7.16 (m, 3H), 7.04 (dd, J = 2.3, 1.0 Hz, 1H), 5.60 (br t, J = 5.4 Hz, 1H), 3.78-3.69 (m, 2H), 3.66 (t, J = 2.7 Hz, 1H), 3.57 (td, J = 6.1, 4.8 Hz, 1H), 3.53 (s, 3H), 3.52-3.46 (m, 1H), 3.42 (s, 1H), 2.95 (s, 3H), 2.61-2.43 (m, 1H), 2.09 (d, J = 1.0 Hz, 3H), 2.07-1.99 (m, 2H), 1.79-1.69 (m, 2H), 1.68-1.61 (m, 2H), 1.59-1.49 (m, 2H). | C |
| 5b | 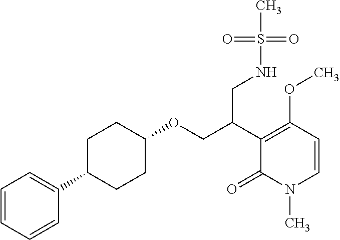 [SMILES: COc1ccn(C)c(=O)c1C(CNS(C)(=O)=O)CO[C@H]1CC[C@@H](c2ccccc2)CC1] | N-[2-(4-methoxy-1- methyl-2-oxo-1,2- dihydropyridin-3-yl)-3- {[(CIS)-4- phenylcyclohexyl]oxy} propyl] methanesulfonamide | 449.2 [M + H]⁺ | ¹H NMR (400 MHz, CDCl₃) δ 7.33-7.28 (m, 2H), 7.28-7.25 (m, 1H), 7.25-7.21 (m, 2H), 7.21-7.15 (m, 1H), 6.08 (d, J = 7.6 Hz, 1H), 5.80-5.65 (m, 1H), 4.08-4.01 (m, 1H), 3.86 (s, 3H), 3.82-3.74 (m, 1H), 3.72-3.63 (m, 2H), 3.62-3.54 (m, 2H), 3.50 (s, 3H), 2.94 (s, 3H), 2.52 (tt, J = 11.8, 3.4 Hz, 1H), 2.10-1.98 (m, 2H), 1.86-1.61 (m, 4H), 1.55-1.45 (m, 2H). | C |
| 5c | 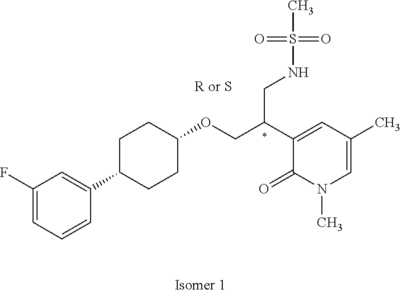 [SMILES: Cc1cc(C(CNS(C)(=O)=O)CO[C@H]2CC[C@@H](c3cccc(F)c3)CC2)c(=O)n(C)c1] | (R or S) N-[2-(1,5- dimethyl-2-oxo-1,2- dihydropyridin-3-yl)-3- {[(CIS)-4-(3- fluorophenyl) cyclohexyl]oxy} propyl] methanesulfonamide— Isomer 1 | 451.2 [M + H]⁺ | ¹H NMR (400 MHz, CDCl₃) δ 7.26-7.20 (m, 2H), 7.07-7.03 (m, 1H), 6.98 (d, J = 7.7 Hz, 1H), 6.91-6.82 (m, 2H), 5.57 (br t, J = 5.2 Hz, 1H), 3.78-3.70 (m, 2H), 3.65 (br s, 1H), 3.61-3.47 (m, 5H), 3.46-3.39 (m, 1H), 2.95 (s, 3H), 2.59-2.46 (m, 1H), 2.12-1.97 (m, 5H), 1.75-1.59 (m, 4H), 1.56-1.46 (m, 2H). | A |
| 5d | 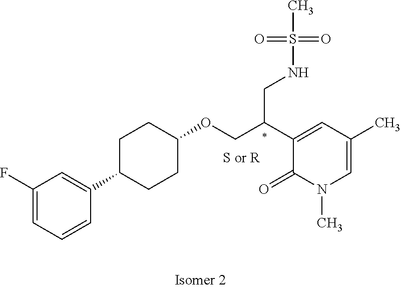 [SMILES: Cc1cc(C(CNS(C)(=O)=O)CO[C@H]2CC[C@@H](c3cccc(F)c3)CC2)c(=O)n(C)c1] | (S or R) N-[2-(1,5- dimethyl-2-oxo-1,2- dihydropyridin-3-yl)-3- {[(CIS)-4-(3- fluorophenyl) cyclohexyl]oxy} propyl] methanesulfonamide— Isomer 2 | 451.2 [M + H]⁺ | ¹H NMR (400 MHz, CDCl₃) δ 7.26-7.18 (m, 2H), 7.08-7.02 (m, 1H), 6.98 (d, J = 7.5 Hz, 1H), 6.92-6.83 (m, 2H), 5.57 (br t, J = 5.3 Hz, 1H), 3.77-3.70 (m, 2H), 3.66 (br d, J = 2.6 Hz, 1H), 3.53 (s, 3H), 3.60-3.47 (m, 2H), 3.43 (q, J = 6.1 Hz, 1H), 2.95 (s, 3H), 2.59-2.47 (m, 1H), 2.09 (s, 3H), 2.04 (br d, J = 12.7 Hz, 2H), 1.77-1.59 (m, 4H), 1.59-1.47 (m, 2H). | B |
| 5e | 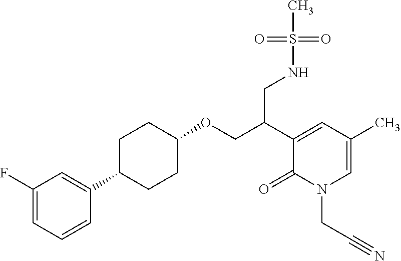 [SMILES: Cc1cc(C(CNS(C)(=O)=O)CO[C@H]2CC[C@@H](c3cccc(F)c3)CC2)c(=O)n(CC#N)c1] | N-{2-[1- (cyanomethyl)-5- methyl-2-oxo-1,2- dihydropyridin-3-yl]-3- {[(CIS)-4-(3- fluorophenyl) cyclohexyl] oxy} propyl }methane- sulfonamide | 476.4 [M + H]⁺ | ¹H NMR (400 MHz, CDCl₃) δ 7.33 (d, J = 2.4 Hz, 1H), 7.26-7.20 (m, 1H), 7.10 (d, J = 1.1 Hz, 1H), 6.98 (d, J = 7.7 Hz, 1H), 6.92-6.80 (m, 2H), 5.29-5.21 (m, 1H), 4.96-4.66 (m, 2H), 3.73 (d, J = 5.9 Hz, 2H), 3.69-3.63 (m, 1H), 3.61-3.47 (m, 2H), 3.47-3.37 (m, 1H), 2.96 (s, 3H), 2.60-2.47 (m, 1H), 2.12 (d, J-0.9 Hz, 3H), 2.09-1.96 (m, 2H), 1.73-1.60 (m, 4H), 1.60-1.39 (m, 2H). | B |
| 5f | 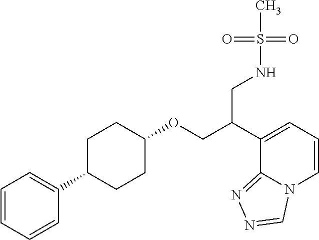 | N-(3-{[(CIS)-4- phenylcyclohexyl] oxy}-2- {[1,2,4]triazolo[4,3- a]pyridin-8-yl}propyl) methanesulfonamide | 447.3 [M + H]⁺ | ¹H NMR (400 MHz, CDCl₃) δ 8.84 (s, 1H), 8.09 (d, J = 6.8 Hz, 1H), 7.30 (d, J = 6.8 Hz, 1H), 7.26-7.19 (m, 1H), 6.95 (d, J = 7.7 Hz, 1H), 6.92-6.83 (m, 2H), 6.79 (br d, J = 10.7 Hz, 1H), 5.49-5.31 (m, 1H), 4.06-3.90 (m, 2H), 3.88-3.80 (m, 3H), 3.67 (br s, 1H), 2.94 (s, 3H), 2.58-2.41 (m, 1H), 2.07-1.94 (m, 2H), 1.66-1.40 (m, 6H). | B |
| 5g | 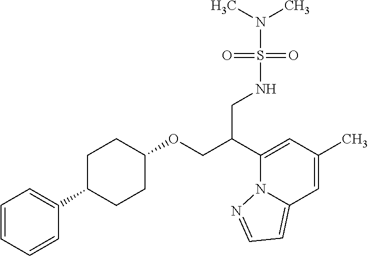 | dimethyl[(2-{5- methylpyrazolo[1,5- a]pyridin-7-yl}-3- {[(CIS)-4- phenylcyclohexyl]oxy} propyl)sulfamoyl] amine | 489.4 [M + H]⁺ | ¹H NMR (400 MHz, CDCl₃) δ 7.91 (d, J = 2.2 Hz, 1H), 7.30-7.26 (m, 1H), 7.24 (d, J = 6.4 Hz, 1H), 6.96 (d, J = 7.5 Hz, 1H), 6.92-6.81 (m, 2H), 6.64 (d, J = 1.3 Hz, 1H), 6.43 (d, J = 2.2 Hz, 1H), 5.36 (br t, J = 5.7 Hz, 1H), 4.15 (quin, J = 5.9 Hz, 1H), 4.02-3.90 (m, 2H), 3.80-3.72 (m, 1H), 3.71-3.61 (m, 2H), 2.68 (s, 6H), 2.51 (tt, J = 10.1, 5.1 Hz, 1H), 2.38 (s, 3H), 2.08-1.96 (m, 2H), 1.67-1.58 (m, 4H), 1.56-1.45 (m, 2H). | B |
| 5h | 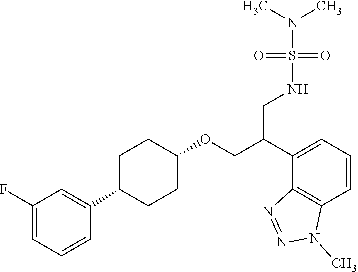 [SMILES: CN(C)S(=O)(=O)NCC(CO[C@H]1CC[C@@H](c2cccc(F)c2)CC1)c1cccc2c1nnn2C] | dimethyl ({[2-(1- methyl-1H-1,2,3- benzotriazol-4-yl)-3- {[(CIS)-4-(3- fluorophenyl) cyclohexyl]oxy} propyl]sulfamoyl}) amine | 490.3 [M + H]⁺ | ¹H NMR (400 MHz, CDCl₃) δ 7.52-7.47 (m, 1H), 7.47-7.44 (m, 1H), 7.33 (dd, J = 6.6, 1.0 Hz, 1H), 7.23 (td, J = 7.9, 6.3 Hz, 1H), 6.94 (d, J = 7.7 Hz, 1H), 6.87 (td, J = 8.4, 1.9 Hz, 1H), 6.77 (dt, J = 10.4, 1.8 Hz, 1H), 5.12 (br t, J = 5.8 Hz, 1H), 4.31 (s, 3H), 4.09-4.02 (m, 1H), 4.00-3.92 (m, 1H), 3.94-3.89 (m, 1H), 3.89-3.72 (m, 2H), 3.67 (br s, 1H), 2.76 (s, 6H), 2.54-2.41 (m, 1H), 2.07-1.97 (m, 2H), 1.66-1.40 (m, 6H). | C |
| 5i | 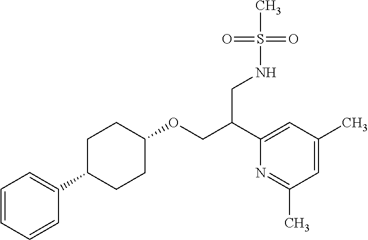 [SMILES: Cc1cc(C)nc(C(CNS(C)(=O)=O)CO[C@H]2CC[C@@H](c3ccccc3)CC2)c1] | N-[2-(4,6- dimethylpyridin-2-yl)- 3-{[(CIS)-4- phenylcyclohexyl]oxy} propyl] methanesulfonamide | 417.3 [M + H]⁺ | ¹H NMR (500 MHz, CDCl₃) δ 7.32-7.28 (m, 2H), 7.22-7.14 (m, 3H), 6.90 (s, 1H), 6.88 (s, 1H), 6.09 (br dd, J = 6.0, 4.9 Hz, 1H), 3.79-3.73 (m, 1H), 3.72-3.68 (m, 1H), 3.68-3.61 (m, 2H), 3.61-3.55 (m, 1H), 3.26 (dq, J = 7.9, 5.6 Hz, 1H), 2.92 (s, 3H), 2.54-2.48 (m, 1H), 2.47 (s, 3H), 2.30 (s, 3H), 2.08-1.95 (m, 2H), 1.80-1.43 (m, 6H). | C |
| 5j | 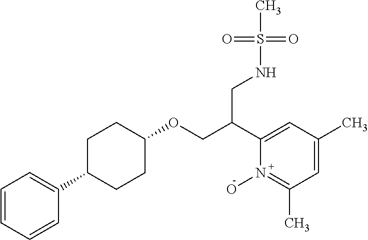 | 2-(1- methanesulfonamido- 3-{[(CIS)-4- phenylcyclohexyl]oxy} propan-2-yl)-4,6- dimethylpyridin-1-ium- 1-olate | 433.2 [M + H]⁺ | ¹H NMR (500 MHz, CDCl₃) δ 7.32-7.28 (m, 2H), 7.23 (d, J = 2.1 Hz, 1H), 7.21-7.17 (m, 3H), 7.05 (d, J = 2.1 Hz, 1H), 6.38 (br t, J = 4.9 Hz, 1H), 4.20-4.12 (m, 1H), 3.83 (d, J = 5.5 Hz, 2H), 3.73-3.65 (m, 2H), 3.62-3.54 (m, 1H), 2.90 (s, 3H), 2.52 (s, 3H), 2.59-2.50 (m, 1H), 2.32 (s, 3H), 2.11-2.00 (m, 2H), 1.80-1.63 (m, 4H), 1.62-1.50 (m, 2H). | A |
| 5k | 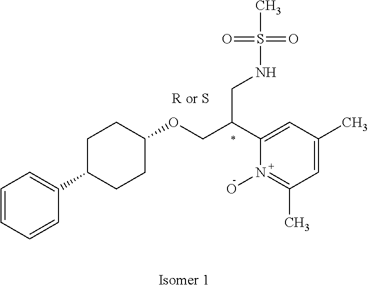 | (R or S) 2-[1- methanesulfonamido- 3-{[(CIS)-4- phenylcyclohexyl]oxy} propan-2-yl]-4,6- dimethylpyridin-1-ium- 1-olate—Isomer 1 | 433.2 [M + H]⁺ | ¹H NMR (400 MHz, CDCl₃) δ 7.30 (dd, J = 8.1, 7.0 Hz, 2H), 7.25-7.15 (m, 4H), 7.05 (s, 1H), 6.36 (d, J = 5.3 Hz, 1H), 4.21-4.10 (m, 1H), 3.84 (d, J = 5.4 Hz, 2H), 3.74-3.65 (m, 2H), 3.64-3.54 (m, 1H), 2.90 (s, 3H), 2.61-2.53 (m, 1H), 2.52 (s, 3H), 2.33 (s, 3H), 2.11-1.99 (m, 2H), 1.80-1.60 (m, 6H). | A |
| 5l | 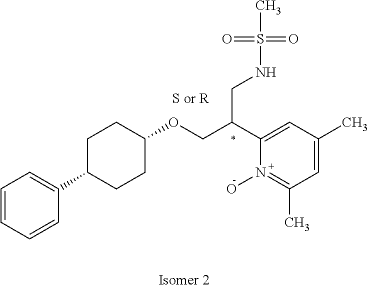 | (S or R) 2-[1- methanesulfonamido- 3-{[(CIS)-4- phenylcyclohexyl]oxy} propan-2-yl]-4,6- dimethylpyridin-1-ium- 1-olate—Isomer 2 | 433.2 [M + H]⁺ | ¹H NMR (400 MHz, CDCl₃) δ 7.34-7.28 (m, 2H), 7.21 (t, J = 7.4 Hz, 4H), 7.05 (s, 1H), 6.36 (s, 1H), 4.21-4.12 (m, 1H), 3.84 (d, J = 5.4 Hz, 2H), 3.69 (q, J = 6.2 Hz, 2H), 3.58 (ddd, J = 12.7, 7.7, 5.7 Hz, 1H), 2.90 (s, 3H), 2.56 (d, J = 11.2 Hz, 1H), 2.52 (s, 3H), 2.33 (s, 3H), 2.04 (d, J = 8.9 Hz, 2H), 1.82-1.58 (m, 6H). | C |
| 5m | 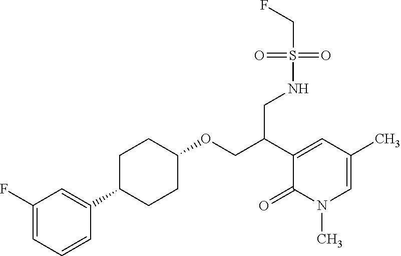 [SMILES: Cc1cc(C(CNS(=O)(=O)CF)CO[C@H]2CC[C@@H](c3cccc(F)c3)CC2)c(=O)n(C)c1] | N-[2-(1,5-dimethyl-2- oxo-1,2- dihydropyridin-3-yl)-3- {[(CIS)-4-(3- fluorophenyl) cyclohexyl]oxy} propyl]-1- fluoromethane- sulfonamide | 469.3 [M + H]⁺ | ¹H NMR (500 MHz, DMSO-d6) δ 7.73 (br t, J = 5.4 Hz, 1H), 7.41 (d, J = 1.1 Hz, 1H), 7.30 (td, J = 7.8, 6.4 Hz, 1H), 7.23 (d, J = 2.5 Hz, 1H), 7.02-6.94 (m, 2H), 6.90 (dt, J = 10.6, 1.9 Hz, 1H), 5.35 (dd, J = 46.4, 10.1 Hz, 1H), 5.31 (dd, J = 46.2, 10.3 Hz, 1H), 3.61 (dd, J = 9.2, 5.8 Hz, 1H), 3.54 (br s, 1H), 3.52 (dd, J = 9.3, 5.6 Hz, 1H), 3.42-3.36 (m, 4H), 3.30-3.26 (m, 1H), 3.25-3.19 (m, 1H), 2.57-2.51 (m, 1H), 2.00 (s, 3H), 1.95-1.82 (m, 2H), 1.62-1.38 (m, 6H). | A |
| 5n | 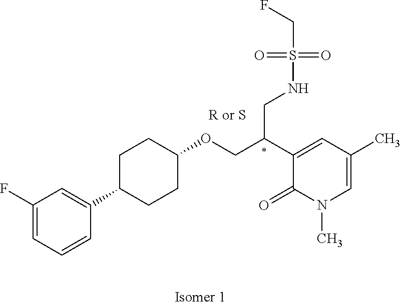 [SMILES: Cc1cc(C(CNS(=O)(=O)CF)CO[C@H]2CC[C@@H](c3cccc(F)c3)CC2)c(=O)n(C)c1] | (R or S) N-[2-(1,5- dimethyl-2-oxo-1,2- dihydropyridin-3-yl)-3- {[(CIS)-4-(3- fluorophenyl) cyclohexyl]oxy} propyl]-1- fluoromethane- sulfonamide—Isomer 1 | 469.3 [M + H]⁺ | ¹H NMR (500 MHz, DMSO-d6) δ 7.73 (br t, J = 5.4 Hz, 1H), 7.41 (d, J = 1.1 Hz, 1H), 7.30 (td, J = 7.8, 6.4 Hz, 1H), 7.23 (d, J = 2.5 Hz, 1H), 7.02-6.94 (m, 2H), 6.90 (dt, J = 10.6, 1.9 Hz, 1H), 5.35 (dd, J = 46.4, 10.1 Hz, 1H), 5.31 (dd, J = 46.2, 10.3 Hz, 1H), 3.61 (dd, J = 9.2, 5.8 Hz, 1H), 3.54 (br s, 1H), 3.52 (dd, J = 9.3, 5.6 Hz, 1H), 3.42-3.36 (m, 4H), 3.30-3.26 (m, 1H), 3.25-3.19 (m, 1H), 2.57-2.51 (m, 1H), 2.00 (s, 3H), 1.95-1.82 (m, 2H), 1.62-1.38 (m, 6H). | A |
| 5o | 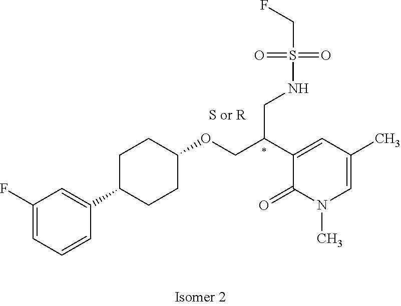 [SMILES: Cc1cc(C(CNS(=O)(=O)CF)CO[C@H]2CC[C@@H](c3cccc(F)c3)CC2)c(=O)n(C)c1] | (S or R) N-[2-(1,5- dimethyl-2-oxo-1,2- dihydropyridin-3-yl)-3- {[(CIS)-4-(3- fluorophenyl) cyclohexyl]oxy} propyl]-1- fluoromethane- sulfonamide—Isomer 2 | 469.3 [M + H]⁺ | ¹H NMR (500 MHz, DMSO-d6) δ 7.73 (br t, J = 5.4 Hz, 1H), 7.41 (d, J = 1.1 Hz, 1H), 7.30 (td, J = 7.8, 6.4 Hz, 1H), 7.23 (d, J = 2.5 Hz, 1H), 7.02-6.94 (m, 2H), 6.90 (dt, J = 10.6, 1.9 Hz, 1H), 5.35 (dd, J = 46.4, 10.1 Hz, 1H), 5.31 (dd, J = 46.2, 10.3 Hz, 1H), 3.61 (dd, J = 9.2, 5.8 Hz, 1H), 3.54 (br s, 1H), 3.52 (dd, J = 9.3, 5.6 Hz, 1H), 3.42-3.36 (m, 4H), 3.30-3.26 (m, 1H), 3.25-3.19 (m, 1H), 2.57-2.51 (m, 1H), 2.00 (s, 3H), 1.95-1.82 (m, 2H), 1.62-1.38 (m, 6H). | B |
| 5p | 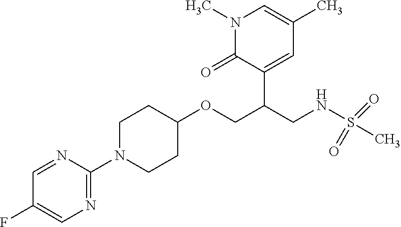 [SMILES: Cc1cc(C(CNS(C)(=O)=O)COC2CCN(c3ncc(F)cn3)CC2)c(=O)n(C)c1] | N-[2-(1,5-dimethyl-2- oxo-1,2- dihydropyridin-3-yl)-3- {[1-(5- fluoropyrimidin-2- yl)piperidin-4- yl]oxy}propyl] methanesulfonamide | 454.4 [M + H]⁺ | ¹H NMR (400 MHz, CDCl₃) δ 8.18 (s, 2H), 7.17 (d, J = 2.2 Hz, 1H), 7.04 (s, 1H), 5.42 (br t, J = 5.4 Hz, 1H), 4.28-4.08 (m, 2H), 3.85-3.73 (m, 2H), 3.53 (s, 3H), 3.63-3.31 (m, 6H), 2.93 (s, 3H), 2.08 (s, 3H), 1.97-1.85 (m, 2H), 1.74-1.44 (m, 2H). | D |
| 5q | 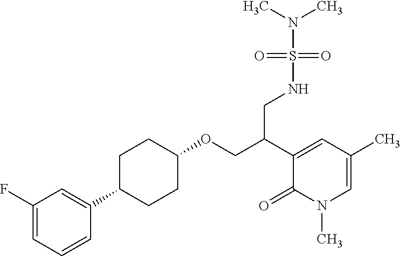 [SMILES: Cc1cc(C(CNS(=O)(=O)N(C)C)CO[C@H]2CC[C@@H](c3cccc(F)c3)CC2)c(=O)n(C)c1] | 3-{1- [(dimethylsulfamoyl) amino]-3-{[(CIS)-4-(3- fluorophenyl) cyclohexyl]oxy} propan-2-yl}-1,5- dimethyl-1,2- dihydropyridin-2-one | 480.3 [M + H]⁺ | ¹H NMR (500 MHz, DMSO-d6) δ 7.40 (d, J = 1.1 Hz, 1H), 7.34-7.27 (m, 1H), 7.21 (d, J = 2.3 Hz, 1H), 7.11 (t, J = 5.8 Hz, 1H), 7.02-6.94 (m, 2H), 6.93-6.87 (m, 1H), 3.58 (dd, J = 9.1, 5.4 Hz, 1H), 3.54 (br s, 1H), 3.48 (dd, J = 9.1, 5.6 Hz, 1H), 3.38 (s, 3H), 3.31-3.28 (m, 1H), 3.27-3.16 (m, 2H), 2.62 (s, 6H), 2.57-2.52 (m, 1H), 2.00 (s, 3H), 1.95-1.82 (m, 2H), 1.62-1.37 (m, 6H). | A |
| 5r | 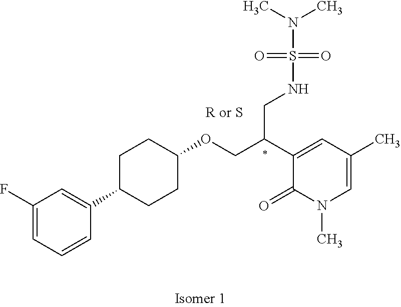 [SMILES: Cc1cc(C(CNS(=O)(=O)N(C)C)CO[C@H]2CC[C@@H](c3cccc(F)c3)CC2)c(=O)n(C)c1] | (R or S) 3-[1- [(dimethylsulfamoyl) amino]-3-{[(CIS)-4-(3- fluorophenyl) cyclohexyl]oxy} propan-2-yl]-1,5- dimethyl-1,2- dihydropyridin-2- one—Isomer 1 | 480.3 [M + H]⁺ | ¹H NMR (500 MHz, DMSO-d6) δ 7.40 (d, J = 1.1 Hz, 1H), 7.35-7.27 (m, 1H), 7.21 (d, J = 2.3 Hz, 1H), 7.11 (t, J = 5.6 Hz, 1H), 7.03-6.94 (m, 2H), 6.94-6.85 (m, 1H), 3.61-3.56 (m, 1H), 3.54 (br s, 1H), 3.48 (dd, J = 9.1, 5.6 Hz, 1H), 3.38 (s, 3H), 3.35-3.29 (m, 1H), 3.27-3.14 (m, 2H), 2.62 (s, 6H), 2.57-2.51 (m, 1H), 2.00 (s, 3H), 1.95-1.81 (m, 2H), 1.64-1.34 (m, 6H). | A |
| 5s | 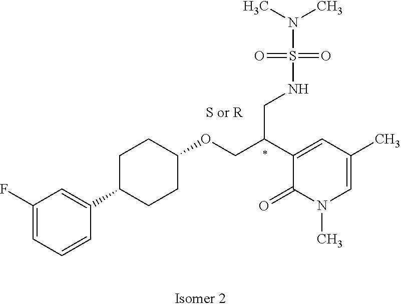 [SMILES: Cc1cc(C(CNS(=O)(=O)N(C)C)CO[C@H]2CC[C@@H](c3cccc(F)c3)CC2)c(=O)n(C)c1] | (S or R) 3-[1- [(dimethylsulfamoyl) amino]-3-{[(CIS)-4-(3- fluorophenyl) cyclohexyl]oxy} propan-2-yl]-1,5- dimethyl-1,2- dihydropyridin-2- one—Isomer 2 | 480.3 [M + H]⁺ | ¹H NMR (500 MHz, DMSO-d6) δ 7.40 (d, J = 1.1 Hz, 1H), 7.35-7.27 (m, 1H), 7.21 (d, J = 2.3 Hz, 1H), 7.11 (t, J = 5.6 Hz, 1H), 7.03-6.94 (m, 2H), 6.94-6.85 (m, 1H), 3.61-3.56 (m, 1H), 3.54 (br s, 1H), 3.48 (dd, J = 9.1, 5.6 Hz, 1H), 3.38 (s, 3H), 3.35-3.29 (m, 1H), 3.27-3.14 (m, 2H), 2.62 (s, 6H), 2.57-2.51 (m, 1H), 2.00 (s, 3H), 1.95-1.81 (m, 2H), 1.64-1.34 (m, 6H). | A |
| 5t | 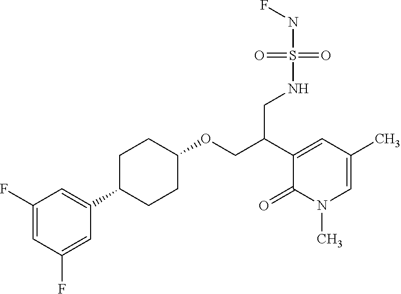 [SMILES: Cc1cc(C(CNS(=O)(=O)NF)CO[C@H]2CC[C@@H](c3cc(F)cc(F)c3)CC2)c(=O)n(C)c1] | N-[2-(1,5-dimethyl-2- oxo-1,2- dihydropyridin-3-yl)-3- {[(CIS)-4-(3,5- difluorophenyl) cyclohexyl]oxy} propyl]-1- fluoromethane- sulfonamide | 487.3 [M + H]⁺ | ¹H NMR (400 MHz, DMSO-d6 ) δ 7.72 (br t, J = 5.6 Hz, 1H), 7.39 (d, J = 0.9 Hz, 1H), 7.23 (d, J = 2.2 Hz, 1H), 7.00 (tt, J = 9.4, 2.2 Hz, 1H), 6.82 (dd, J = 8.9, 1.9 Hz, 2H), 5.44-5.18 (m, 2H), 3.61 (dd, J = 9.2, 5.7 Hz, 1H), 3.57-3.48 (m, 2H), 3.45-3.34 (m, 4H), 3.30-3.16 (m, 2H), 2.63-2.53 (m, 1H), 2.00 (s, 3H), 1.95-1.79 (m, 2H), 1.63-1.31 (m, 6H). | A |
| 5u | 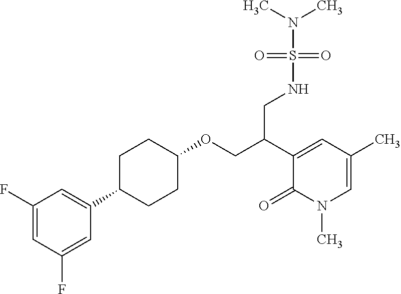 [SMILES: Cc1cc(C(CNS(=O)(=O)N(C)C)CO[C@H]2CC[C@@H](c3cc(F)cc(F)c3)CC2)c(=O)n(C)c1] | 3-{1- [(dimethylsulfamoyl) amino]-3-{[(CIS)-4- (3,5-difluorophenyl) cyclohexyl]oxy} propan-2-yl}-1,5- dimethyl-1,2- dihydropyridin-2-one | 498.3 [M + H]⁺ | ¹H NMR (400 MHz, DMSO-d6 ) δ 7.38 (d, J = 1.1 Hz, 1H), 7.21 (d, J = 2.4 Hz, 1H), 7.11 (br t, J = 5.5 Hz, 1H), 7.00 (tt, J = 9.4, 2.2 Hz, 1H), 6.82 (dd, J = 9.0, 2.0 Hz, 2H), 3.61-3.55 (m, 1H), 3.53 (br s, 1H), 3.48 (dd, J = 9.0, 5.5 Hz, 1H), 3.38 (s, 3H), 3.31-3.12 (m, 3H), 2.62 (s, 6H), 2.59-2.52 (m, 1H), 2.00 (s, 3H), 1.95-1.78 (m, 2H), 1.60-1.35 (m, 6H). | A |
| 5v | 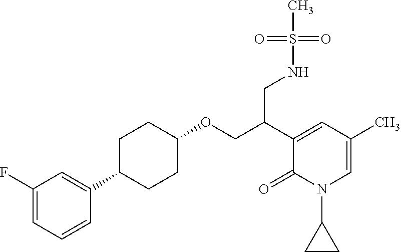 [SMILES: Cc1cc(C(CNS(C)(=O)=O)CO[C@H]2CC[C@@H](c3cccc(F)c3)CC2)c(=O)n(C2CC2)c1] | N-[2-(1-cyclopropyl-5- methyl-2-oxo-1,2- dihydropyridin-3-yl)-3- {[(CIS)-4-(3- fluorophenyl) cyclohexyl]oxy} propyl] methanesulfonamide | 477.3 [M + H]⁺ | ¹H NMR (400 MHz, CDCl₃) δ 7.24 (br d, J = 6.6 Hz, 1H), 7.21 (d, J = 1.8 Hz, 1H), 7.05 (s, 1H), 6.97 (d, J = 7.7 Hz, 1H), 6.93-6.80 (m, 2H), 5.54 (br t, J = 5.2 Hz, 1H), 3.79-3.69 (m, 2H), 3.65 (br s, 1H), 3.61-3.46 (m, 2H), 3.46-3.37 (m, 1H), 3.34-3.23 (m, 1H), 2.95 (s, 3H), 2.59-2.47 (m, 1H), 2.08 (s, 3H), 2.03 (br d, J = 12.7 Hz, 2H), 1.74-1.45 (m, 6H), 1.17-1.08 (m, 2H), 0.89-0.80 (m, 2H). | A |
| 5w | 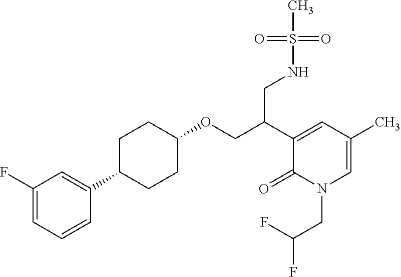 [SMILES: Cc1cc(C(CNS(C)(=O)=O)CO[C@H]2CC[C@@H](c3cccc(F)c3)CC2)c(=O)n(CC(F)F)c1] | N-{2-[1-(2,2- difluoroethyl)-5- methyl-2-oxo-1,2- dihydropyridin-3-yl]-3- {[(CIS)-4-(3- fluorophenyl) cyclohexyl]oxy} propyl }methane- sulfonamide | 501.3 [M + H]⁺ | ¹H NMR (500 MHz, CDCl₃) δ 7.31 (d, J = 2.3 Hz, 1H), 7.27-7.21 (m, 1H), 7.06-6.94 (m, 2H), 6.92-6.81 (m, 2H), 6.11 (tt, J = 56.1, 4.4 Hz, 1H), 5.43 (br t, J = 5.6 Hz, 1H), 4.22 (tdd, J = 13.4, 13.4, 4.4, 1.2 Hz, 2H), 3.73 (d, J = 5.8 Hz, 2H), 3.68-3.63 (m, 1H), 3.59-3.53 (m, 1H), 3.53-3.46 (m, 1H), 3.45-3.37 (m, 1H), 2.95 (s, 3H), 2.54 (tt, J = 10.3, 5.2 Hz, 1H), 2.10 (d, J = 1.0 Hz, 3H), 2.08-1.97 (m, 2H), 1.84-1.45 (m, 6H). | A |
| 5x | 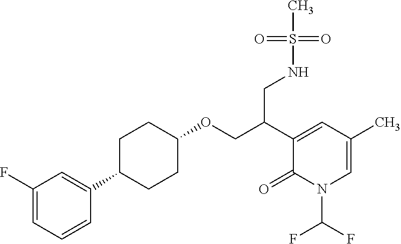 [SMILES: Cc1cc(C(CNS(C)(=O)=O)CO[C@H]2CC[C@@H](c3cccc(F)c3)CC2)c(=O)n(C(F)F)c1] | N-{2-[1- (difluoromethyl)-5- methyl-2-oxo-1,2- dihydropyridin-3-yl]-3- {[(CIS)-4-(3- fluorophenyl) cyclohexyl]oxy} propyl }methane- sulfonamide | 487.3 [M + H]⁺ | ¹H NMR (500 MHz, CDCl₃) δ 7.68 (t, J = 60.4 Hz, 1H), 7.32 (d, J = 2.3 Hz, 1H), 7.27-7.22 (m, 1H), 7.20 (s, 1H), 6.98 (d, J = 7.7 Hz, 1H), 6.93-6.79 (m, 2H), 5.17 (br t, J = 5.8 Hz, 1H), 3.72 (d, J = 5.8 Hz, 2H), 3.69-3.63 (m, 1H), 3.61-3.47 (m, 2H), 3.40 (quin, J = 6.0 Hz, 1H), 2.95 (s, 3H), 2.60-2.46 (m, 1H), 2.12 (d, J = 1.0 Hz, 3H), 2.03 (ddt, J = 14.1, 5.7, 2.6, 2.6 Hz, 2H), 1.79-1.62 (m, 4H), 1.61-1.46 (m, 2H). | A |
| 5y | 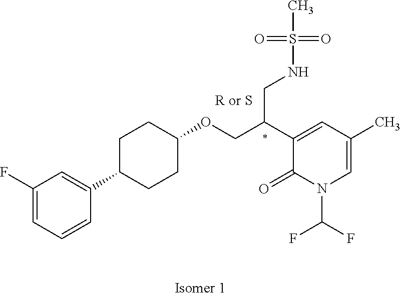 [SMILES: Cc1cc(C(CNS(C)(=O)=O)CO[C@H]2CC[C@@H](c3cccc(F)c3)CC2)c(=O)n(C(F)F)c1] | (R or S) N-[2-[1- (difluoromethyl)-5- methyl-2-oxo-1,2- dihydropyridin-3-yl]-3- {[(CIS)-4-(3- fluorophenyl) cyclohexyl] oxy}propyl] methanesulfonamide— Isomer 1 | 487.3 [M + H]⁺ | ¹H NMR (500 MHz, DMSO-d6 ) δ 8.02-7.71 (m, 1H), 7.49 (s, 1H), 7.37 (d, J = 2.3 Hz, 1H), 7.30 (td, J = 7.9, 6.4 Hz, 1H), 7.05-6.93 (m, 3H), 6.90 (dt, J = 10.5, 2.1 Hz, 1H), 3.63-3.57 (m, 1H), 3.47-3.57 (m, 2H), 3.36-3.30 (m, 1H), 3.29-3.19 (m, 2H), 2.87 (s, 3H), 2.57-2.51 (m, 1H), 2.05 (d, J = 1.1 Hz, 3H), 1.96-1.80 (m, 2H), 1.60-1.38 (m, 6H) | A |
| 5z | 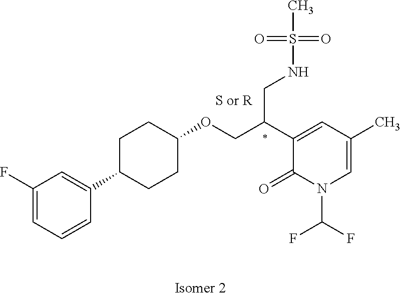 [SMILES: Cc1cc(C(CNS(C)(=O)=O)CO[C@H]2CC[C@@H](c3cccc(F)c3)CC2)c(=O)n(C(F)F)c1] | (S or R) N-[2-[1- (difluoromethyl)-5- methyl-2-oxo-1,2- dihydropyridin-3-yl]-3- {[(CIS)-4-(3- fluorophenyl) cyclohexyl]oxy} propyl]methane- sulfonamide—Isomer 2 | 487.3 [M + H]⁺ | ¹H NMR (400 MHz, CDCl₃) δ 7.91-7.45 (m, 1H), 7.32 (d, J = 2.2 Hz, 1H), 7.27-7.21 (m, 1H), 7.20 (s, 1H), 6.98 (d, J = 7.7 Hz, 1H), 6.91-6.83 (m, 2H), 5.20 (br t, J = 5.7 Hz, 1H), 3.72 (d, J = 5.5 Hz, 2H), 3.68-3.62 (m, 1H), 3.61-3.46 (m, 2H), 3.45-3.36 (m, 1H), 2.95 (s, 3H), 2.63-2.44 (m, 1H), 2.12 (s, 3H), 2.03 (br dd, J = 16.2, 2.6 Hz, 2H), 1.78-1.43 (m, 6H). | C |
| 5aa | 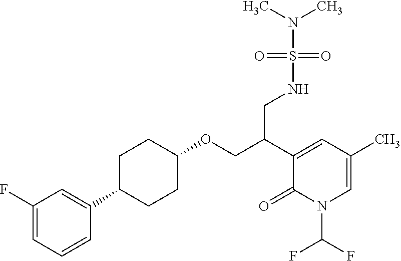 [SMILES: Cc1cc(C(CNS(=O)(=O)N(C)C)CO[C@H]2CC[C@@H](c3cccc(F)c3)CC2)c(=O)n(C(F)F)c1] | 1-(difluoromethyl)-3- {1- [(dimethylsulfamoyl) amino]-3-{[(CIS)-4-(3- fluorophenyl) cyclohexyl] oxy}propan-2-yl}-5- methyl-1,2- dihydropyridin-2-one | 516.3 [M + H]⁺ | ¹H NMR (400 MHz, CDCl₃) δ 7.69 (t, J = 60.5 Hz, 1H), 7.33 (d, J = 2.3 Hz, 1H), 7.26-7.20 (m, 1H), 7.19 (s, 1H), 6.98 (d, J = 7.8 Hz, 1H), 6.93-6.83 (m, 2H), 5.14-5.01 (m, 1H), 3.79-3.61 (m, 3H), 3.58-3.35 (m, 3H), 2.78 (s, 6H), 2.59-2.47 (m, 1H), 2.12 (s, 3H), 2.08-1.94 (m, 2H), 1.80-1.46 (m, 6H). | A |
| 5ab | 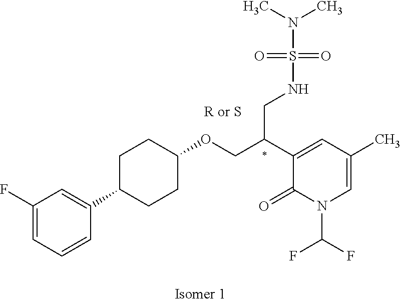 [SMILES: Cc1cc(C(CNS(=O)(=O)N(C)C)CO[C@H]2CC[C@@H](c3cccc(F)c3)CC2)c(=O)n(C(F)F)c1] | (R or S) 1- (difluoromethyl)-3-[1- [(dimethylsulfamoyl) amino]-3-{[(CIS)-4-(3- fluorophenyl) cyclohexyl]oxy} propan-2-yl]-5-methyl- 1,2-dihydropyridin-2- one—Isomer 1 | 516.3 [M + H]⁺ | ¹H NMR (400 MHz, CDCl₃) δ 7.68 (t, J = 60.5 Hz, 1H), 7.33 (d, J = 2.2 Hz, 1H), 7.26-7.21 (m, 1H), 7.19 (s, 1H), 6.98 (d, J = 7.7 Hz, 1H), 6.92-6.84 (m, 2H), 5.07 (br t, J = 5.3 Hz, 1H), 3.75-3.62 (m, 3H), 3.57-3.47 (m, 1H), 3.46-3.37 (m, 2H), 2.79 (s, 6H), 2.59-2.48 (m, 1H), 2.12 (s, 3H), 2.08-1.96 (m, 2H), 1.78-1.62 (m, 4H), 1.57-1.46 (m, 2H). | A |
| 5ac | 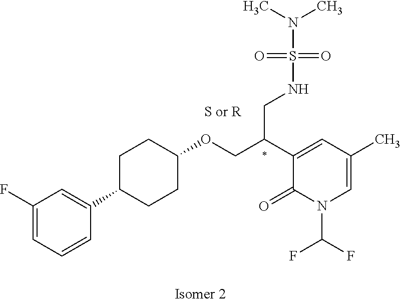 [SMILES: Cc1cc(C(CNS(=O)(=O)N(C)C)CO[C@H]2CC[C@@H](c3cccc(F)c3)CC2)c(=O)n(C(F)F)c1] | (S or R) 1- (difluoromethyl)-3-[1- [(dimethylsulfamoyl) amino]-3-{[(CIS)-4-(3- fluorophenyl) cyclohexyl]oxy} propan-2-yl]-5-methyl- 1,2-dihydropyridin-2- one—Isomer 2 | 516.3 [M + H]⁺ | ¹H NMR (400 MHz, CDCl₃) δ 7.68 (t, J = 60.5 Hz, 1H), 7.32 (d, J = 2.4 Hz, 1H), 7.26-7.19 (m, 1H), 7.18 (d, J = 2.4 Hz, 1H), 6.97 (dt, J = 7.7, 1.4 Hz, 1H), 6.90-6.83 (m, 2H), 5.04 (d, J = 6.1 Hz, 1H), 3.74-3.67 (m, 2H), 3.66-3.61 (m, 1H), 3.55-3.46 (m, 1H), 3.45-3.36 (m, 2H), 2.78 (s, 6H), 2.53 (tt, J = 10.4, 5.5 Hz, 1H), 2.11 (d, J = 1.2 Hz, 3H), 2.08-1.96 (m, 2H), 1.66 (dddd, J = 11.1, 8.7, 6.6, 3.0 Hz, 4H), 1.56 (s, 2H). | B |

### Example 6a: N-(2-{5-methyl-6-oxo-1,6-dihydro-[3,3′-bipyridin]-1-yl}-3-{[(CIS)-4-phenylcyclohexyl]oxy}propyl) methanesulfonamide (6a)

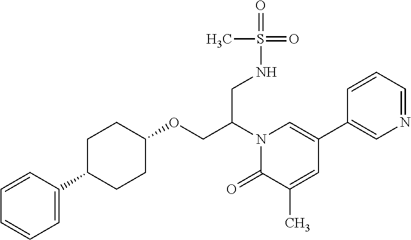 [SMILES: Cc1cc(-c2cccnc2)cn(C(CNS(C)(=O)=O)CO[C@H]2CC[C@@H](c3ccccc3)CC2)c1=O]

To a mixture of Example 2X (40 mg, 0.080 mmol), Cs₂CO₃ (105 mg, 0.32 mmol), Pd(PPh₃)₄ (28 mg, 0.020 mmol) and 3-(4,4,5,5-tetramethyl-1,3,2-dioxaborolan-2-yl)pyridine (66 mg, 0.32 mmol), DMF (2 mL) was added. The suspension was degassed by bubbling N₂ for 10 min, then it was stirred at 120° C. for 16 h. The mixture was diluted with EtOAc and washed with H₂O and brine. The organic layer was evaporated in vacuo. The product was purified by column chromatography using a C₁₈ cartridge (H₂O+formic acid 0.1%/MeCN+formic acid 0.1%, 95:5 to 40:60) followed by preparative HPLC (MDAP Waters with mass spectrometry detection MS:ZQ2000, column: xBridge C₁₈ (30×100 mm, 3 μm), conditions: [A2: 10 mM NH₄HCO₃ aq. sol. adjusted to pH 10 with NH₃]; [B2: MeCN], gradient: from 43.0% B2 to 45.0% B2 in 10 min, flow: 40.00 mL/min) to afford the title compound (12 mg, 0.024 mmol, 30% yield) as a white solid.

TABLE 9
Characterization and EC50 hOX2 data—Compound 6a

| | | | Obs. | | EC50 |
|---|---|---|---|---|---|
| Ex. | Structure | Name | Mass | ¹H NMR | hOX2 |
| 6a | 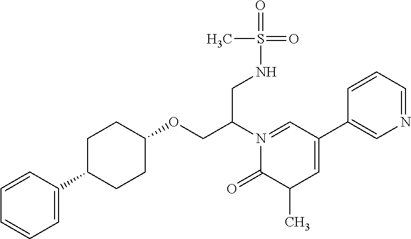 | N-(2-{5-methyl-6- oxo-1,6-dihydro- [3,3′-bipyridin]-1- yl}-3-{[(CIS)-4- phenylcyclohexyl] oxy} propyl)methane- sulfonamide | 496.2 [M + H]⁺ | ¹H NMR (500 MHz, CDCl₃) δ 8.72 (dd, J = 2.3, 0.7 Hz, 1H), 8.55 (dd, J = 4.8, 1.6 Hz, 1H), 7.82 (d, J = 2.2 Hz, 1H), 7.76-7.67 (m, 1H), 7.53 (dd, J = 2.5, 1.2 Hz, 1H), 7.27-7.24 (m, 1H), 7.24-7.19 (m, 2H), 7.18-7.13 (m, 1H), 7.08-7.01 (m, 2H), 5.23-5.12 (m, 2H), 3.97 (dd, J = 10.4, 5.5 Hz, 1H), 3.90-3.79 (m, 2H), 3.72 (dt, J = 13.7, 5.5 Hz, 1H), 3.67 (br s, 1H), 2.93 (s, 3H), 2.55-2.45 (m, 1H), 2.24 (s, 3H), 2.08-1.94 (m, 2H), 1.69-1.50 (m, 6H). | C |

### Example 7a: N-{2-[3-methyl-2-oxo-5-(1H-pyrazol-1-yl)-1,2-dihydropyridin-1-yl]-3-{[(CIS)-4-phenylcyclohexyl]oxy}propyl}methanesulfonamide (7a)

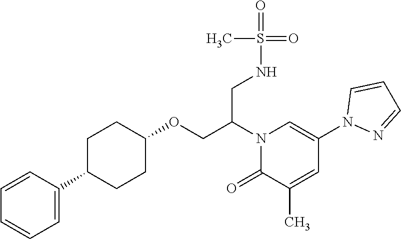 [SMILES: Cc1cc(-n2cccn2)cn(C(CNS(C)(=O)=O)CO[C@H]2CC[C@@H](c3ccccc3)CC2)c1=O]

To a mixture of Example 2X (40 mg, 0.080 mmol), pyrazole (22 mg, 0.32 mmol), Cu₂O (1 mg, 0.010 mmol), and Cs₂CO₃ (105 mg, 0.32 mmol), DMSO (2 mL) was added. The suspension was degassed by bubbling N₂ for 10 min, then it was stirred at 120° C. for 16 h. The mixture was diluted with EtOAc and washed with H₂O and brine. The organic layer was evaporated in vacuo. The product was purified by column chromatography using a silica cartridge (cHex/EtOAc, from 40:60 to 100:0) followed by preparative HPLC (MDAP Waters with mass spectrometry detection MS:ZQ2000, column: xBridge C18 (30×100 mm, 3 μm), conditions: [A2: 10 mM NH₄HCO₃ aq. sol. adjusted to pH 10 with NH₃]; [B2: MeCN], gradient: from 45.0% B2 to 446.0% B2 in 10 min, flow: 40.00 mL/min) to afford the title compound (12 mg, 0.024 mmol, 30% yield) as a white solid.

TABLE 10
Characterization and EC50 hOX2 data—Compound 7a

| | | | Obs. | | EC50 |
|---|---|---|---|---|---|
| Ex. | Structure | Name | Mass | ¹H NMR | hOX2 |
| 7a | 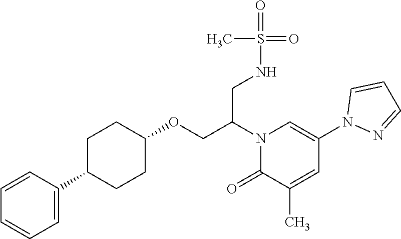 [SMILES: Cc1cc(-n2cccn2)cn(C(CNS(C)(=O)=O)CO[C@H]2CC[C@@H](c3ccccc3)CC2)c1=O] | N-{2-[3-methyl-2-oxo- 5-(1H-pyrazol-1-yl)- 1,2-dihydropyridin-1- yl]-3-{[(CIS)-4- phenylcyclohexyl]oxy} propyl }methane- sulfonamide | 485.2 [M + H]⁺ | ¹H NMR (500 MHz, CDCl₃) δ 8.06 (d, J = 2.6 Hz, 1H), 7.69 (d, J = 2.5 Hz, 1H), 7.66-7.59 (m, 2H), 7.27-7.22 (m, 2H), 7.21-7.15 (m, 1H), 7.14-7.07 (m, 2H), 6.40 (t, J = 2.1 Hz, 1H), 5.25-5.19 (m, 1H), 5.16 (t, J = 6.2 Hz, 1H), 3.98-3.91 (m, 1H), 3.90-3.85 (m, 1H), 3.82-3.70 (m, 2H), 3.67 (t, J = 2.7 Hz, 1H), 2.93 (s, 3H), 2.56-2.47 (m, 1H), 2.24 (s, 3H), 2.08-1.97 (m, 2H), 1.72-1.50 (m, 6H). | B |

### Example 8a: N-(3-{[1,1′-biphenyl]-3-yl}-2-(2-oxo-1,2-dihydropyridin-1-yl)propyl)methanesulfonamide (8a)

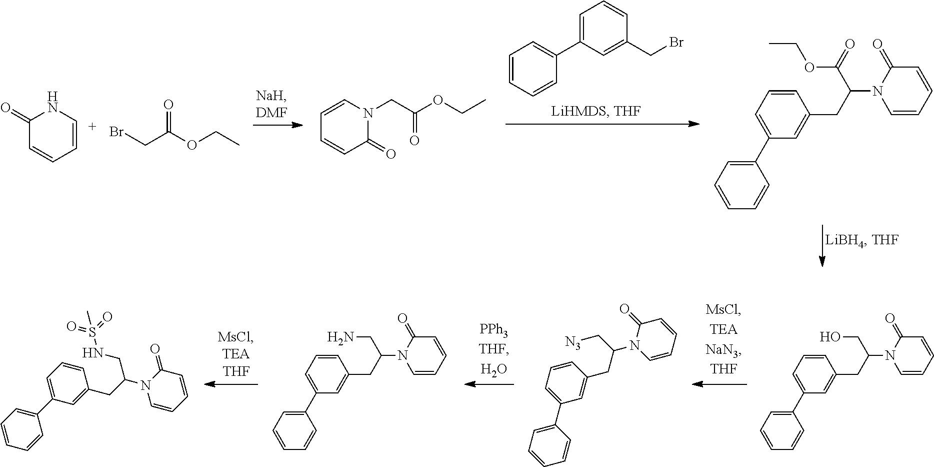

### Ethyl 2-(2-oxo-1,2-dihydropyridin-1-yl)acetate (INTERMEDIATE 23)

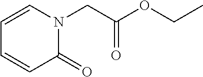

To a suspension of NaH (0.93 g, 38.92 mmol) in anhydrous DMF (40 mL) was added 2-pyridone (3.42 g, 35.93 mmol). The mixture was stirred for 10 minutes then it was added to a solution of ethyl 2-bromoacetate (5.0 g, 29.94 mmol) in anhydrous DMF (35 mL). The reaction mixture was stirred for 72 h at room temperature, then it was diluted with DCM and washed with sat. aq. NaHCO₃ and brine. The organic layer was dried with a phase separator and evaporated in vacuo. The product was purified by column chromatography using a silica cartridge (0-100% EtOAc in cHex) to afford the title compound (5.56 g, quant. yield) as a pale-yellow oil. [M+H]+m/z 182.0

### Ethyl 3-{[1,1′-biphenyl]-3-yl}-2-(2-oxo-1,2-dihydropyridin-1-yl)propanoate (INTERMEDIATE 24)

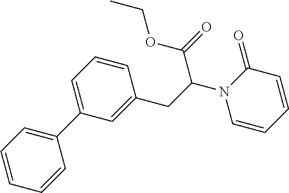 [SMILES: CCOC(=O)C(Cc1cccc(-c2ccccc2)c1)n1ccccc1=O]

To a solution of Intermediate 23 (3.0 g, 16.56 mmol) in THF (83 mL), a 1M solution of LiHMDS in hexanes (24.84 mL, 24.84 mmol) was added at −78° C. After 15 minutes, 3-(bromomethyl)-1,1′-biphenyl (5.32 g, 21.52 mmol) was added and mixture was stirred at −78° C. for 90 minutes. A saturated solution of NH₄Cl(30 mL) was added and the reaction was allowed to warm to room temperature. The solution was acidified by addition of 1M HCl and extracted with EtOAc (3×250 mL). The organic layer was dried over a phase separator and evaporated in vacuo. The product was purified by column chromatography using a silica cartridge (0-50% EtOAc in cHex) to afford the title compound (5.2 g, 14.97 mmol, 90% yield) as pale-yellow oil. [M+H]⁺ m/z 348.2

### 1-(1-{[1,1′-Biphenyl]-3-yl}-3-hydroxypropan-2-yl)-1,2-dihydropyridin-2-one (INTERMEDIATE 25)

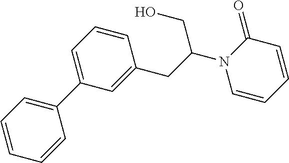 [SMILES: O=c1ccccn1C(CO)Cc1cccc(-c2ccccc2)c1]

To a solution of Intermediate 24 (2.0 g, 5.76 mmol) in THF (41 mL) at −40° C., a 2M solution of LiBH₄ in THF (4.32 mL, 8.64 mmol) was added. The mixture was stirred at −40° C. for 1 h and at room temperature for 24 h. The reaction mixture was quenched with water (50 mL) and 1M aqueous NaOH (25 mL) and extracted with EtOAc (3×200 mL), then the combined organic layers were dried (Na₂SO₄) and evaporated in vacuo to afford the title product (2 g, quant. yield) as a pale-yellow oil. [M+H]⁺ m/z 306.2

### 1-(1-Azido-3-{[1,1′-biphenyl]-3-yl}propan-2-yl)-1,2-dihydropyridin-2-one (INTERMEDIATE 26)

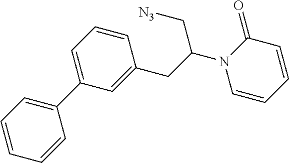 [SMILES: [N-]=[N+]=NCC(Cc1cccc(-c2ccccc2)c1)n1ccccc1=O]

To a solution of Intermediate 25 (100 mg, 0.33 mmol) in THF (3 mL) at 0° C., TEA (68 μL, 0.49 mmol) and MsCl (27 μL, 0.34 mmol) were added. After 30 minutes, NaN₃ (64 mg, 0.98 mmol) and DMF (0.1 mL) were added at 0° C. The mixture was stirred at 60° C. for 1 h. The mixture was diluted with H₂O (10 mL) and extracted with EtOAc (3×50 mL). The combined organic layer was evaporated in vacuo and the product was purified by column chromatography using a silica cartridge (0-30% EtOAc in cHex) to afford the title compound (46 mg, 0.14 mmol, 42% yield) as a colourless oil. [M+H]⁺ m/z 331.2

### 1-(1-Amino-3-{[1,1′-biphenyl]-3-yl}propan-2-yl)-1,2-dihydropyridin-2-one (INTERMEDIATE 27)

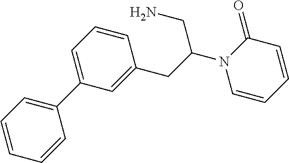 [SMILES: NCC(Cc1cccc(-c2ccccc2)c1)n1ccccc1=O]

To a solution of PPh₃ (73 mg, 0.28 mmol) in THF (0.5 mL) and H₂O (0.25 mL), Intermediate 27 (46 mg, 0.14 mmol) was added. The mixture was stirred at room temperature for 16 h. H₂O (10 mL) was added and the product was extracted with Et₂O (3×50 mL). The organic layer was dried (Na₂SO₄) and evaporated in vacuo. The product was loaded into an SCX cartridge as a MeOH solution, then it was washed with MeOH and eluted with a NH₃ solution in MeOH to afford the title compound (18 mg, 0.059 mmol, 42% yield) as a colourless oil. [M+H]⁺ m/z 305.3

### N-(3-{[1,1′-biphenyl]-3-yl}-2-(2-oxo-1,2-dihydropyridin-1-yl)propyl)methanesulfonamide (8a)

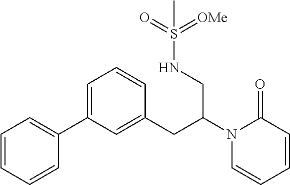

Example 8a was prepared following the procedure described for Example 1, starting from Intermediate 27 (18 mg, 0.059 mmol) to afford the title compound (5 mg, 0.013 mmol, 22% yield) as a colourless oil.

TABLE 11
Characterization and EC50 hOX2 data—Compound 8a

| | | | Obs. | | EC50 |
|---|---|---|---|---|---|
| Ex. | Structure | Name | Mass | ¹H NMR | hOX2 |
| 8a | 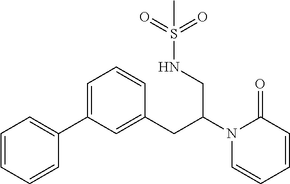 [SMILES: CS(=O)(=O)NCC(Cc1cccc(-c2ccccc2)c1)n1ccccc1=O] | N-(3-{[1,1′-biphenyl]-3-yl}-2- (2-oxo-1,2-dihydropyridin-1- yl)propyl)methanesulfonamide | 383.2 [M + H]⁺ | ¹H NMR (400 MHz, CDCl₃) δ 7.56-7.49 (m, 2H), 7.48-7.29 (m, 7H), 7.13 (d, J = 7.5 Hz, 1H), 7.06 (br d, J = 6.6 Hz, 1H), 6.57 (d, J = 8.3 Hz, 1H), 6.12 (td, J = 6.7, 1.1 Hz, 1H), 5.01-4.82 (m, 2H), 3.91-3.79 (m, 1H), 3.65-3.55 (m, 1H), 3.36 (dd, J = 14.0, 8.8 Hz, 1H), 3.17 (dd, J = 13.9, 6.5 Hz, 1H), 2.89 (s, 3H). | D |

### EMBODIMENTS

1. A compound of Formula (I):

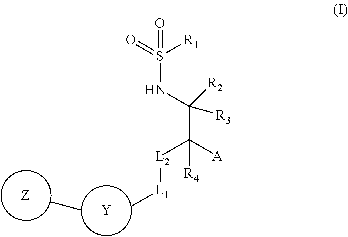

or a pharmaceutically acceptable salt thereof,
wherein
A is a heteroaryl;
L₁ is —O—, —CR₅R₆— or a bond;
L₂ is —CR₅R₆;
R₁ is alkyl, cycloalkyl, heterocyclyl, aryl, heteroaryl, or —NR₇R₈, or R₁ and R₂ together with the atom to which they are attached form a heterocycle;
R₂, R₃, R₄ are independently hydrogen, alkyl, cycloalkyl, heterocyclyl, or halogen, or R₂ and R₃ together with the atom to which they are attached to form a carbocycle or heterocycle, or R₂ and R₄ together with the atom to which they are attached to form a carbocycle or heterocycle;
R₅ and R₆ are each independently hydrogen, alkyl, cycloalkyl, heterocyclyl, alkoxy, —O-cycloalkyl, —O-heterocyclyl, or halogen, or R₅ and R₆ together with the atom to which they are attached to form a carbocycle or heterocycle;
R₇ and R₈ are independently hydrogen, alkyl, cycloalkyl, or heterocyclyl, or R₇ and R₈ together with the atom to which they are attached form a heterocycle;
Y is cycloalkyl, heterocyclyl, heteroaryl or aryl; and
Z is absent, heteroaryl or aryl, wherein if L₁ is a bond or CR₅R₆ then Z is heteroaryl or aryl; and with the proviso that the compound is not:

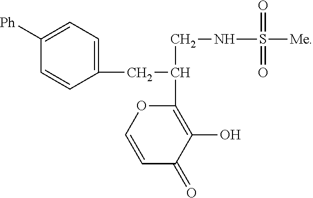 [SMILES: CS(=O)(=O)NCC(Cc1ccc(-c2ccccc2)cc1)c1occc(=O)c1O]

2. The compound of embodiment 1, wherein L₁ is —O—.
3. The compound of embodiment 1, wherein L₁ is a bond.
4. The compound of any one of embodiments 1-3, wherein R₁ is an alkyl.
5. The compound of embodiment 4, wherein R₁ is methyl.
6. The compound of any one of embodiments 1-3, wherein R₁ is —NR₇R₈.
7. The compound of embodiment 6, wherein R₇ and R₈ are alkyl.
8. The compound of any one of embodiments 1-7, wherein R₂, R₃, R₄ are hydrogen.
9. The compound of any one of embodiments 1-8, wherein Y is 3-7 membered monocyclic cycloalkyl, 5-8 membered bicyclic cycloalkyl, 4-7 membered saturated heterocyclyl, 5-8 membered bicyclic heterocyclyl, 5-6-membered heteroaryl or phenyl.
10. The compound of any one of embodiments 1-9, wherein Z is 5-10 membered heteroaryl or phenyl.
11. The compound of any one of embodiments 1-8, wherein Y and Z are phenyl.
12. The compound of any one of embodiments 1-8, wherein Y is cyclohexyl and Z is phenyl.
13. The compound of embodiment 11, wherein Y and Z together are:

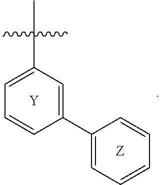

14. The compound of any one of embodiments 1-13, wherein A is a substituted or unsubstituted monocyclic or bicyclic nitrogen-containing heteroaryl.
15. The compound of embodiment 14, wherein A is selected from the group consisting of:

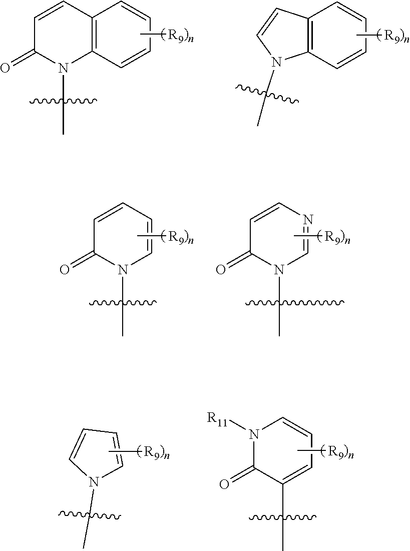

wherein n is an integer from 0-6;
R₉ is hydrogen, alkyl, halogen, cyano, alkoxy, cycloalkyl, heterocyclyl, or heteroaryl; and
R₁₁ is alkyl, cycloalkyl or heterocyclyl.
16. The compound of embodiment 1, wherein the compound of Formula (I) is selected from the group consisting of compounds in Table 1.
17. A pharmaceutical composition comprising a compound of any one of embodiments 1-16 and pharmaceutically acceptable excipient.
18. A method of treating a disease or disorder that is treatable by administration of an Orexin agonist, the method comprising administering a therapeutically effective amount of the compound of any one of embodiments 1-16 or the composition of embodiment 17.
19. A method of treating a sleep disorder in a subject in need thereof, comprising administering to the subject a therapeutically effective amount of the compound of any one of embodiments 1-16 or the composition of embodiment 17.
20. A method for treating narcolepsy in a subject in need thereof, comprising administering to the subject an effective amount of the compound of the compound of any one of embodiments 1-16 or the composition of embodiment 17.
21. A method for treating hypersomnia in a subject in need thereof, comprising administering to the subject an effective amount of the compound of the compound of any one of embodiments 1-16 or the composition of embodiment 17.
22. A method for decreasing or treating excessive sleepiness in a subject in need thereof, comprising administering to the subject an effective amount of the compound of the compound of any one of embodiments 1-16 or the composition of embodiment 17.

1. A compound of Formula (I): 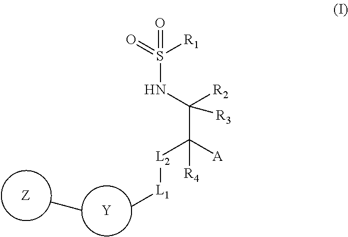
or a pharmaceutically acceptable salt thereof,
wherein
A is a heteroaryl;
L₁ is —O—, —CR₅R₆—or a bond;
L₂ is —CR₅R₆;
R₁ is alkyl, cycloalkyl, heterocyclyl, aryl, heteroaryl, or —NR₇R₈, or R₁ and R₂ together with the atom to which they are attached form a heterocycle;
R₂, R₃, R₄ are independently hydrogen, alkyl, cycloalkyl, heterocyclyl, or halogen, or R₂ and R₃ together with the atom to which they are attached to form a carbocycle or heterocycle, or R₂ and R₄ together with the atom to which they are attached to form a carbocycle or heterocycle;
R₅ and R₆ are each independently hydrogen, alkyl, cycloalkyl, heterocyclyl, alkoxy, —O-cycloalkyl, —O-heterocyclyl, or halogen, or R₅ and R₆ together with the atom to which they are attached to form a carbocycle or heterocycle;
R₇ and R₈ are independently hydrogen, alkyl, cycloalkyl, or heterocyclyl, or R₇ and R₈ together with the atom to which they are attached form a heterocycle;
Y is cycloalkyl, heterocyclyl, heteroaryl or aryl; and
Z is absent, heteroaryl or aryl, wherein if L₁ is a bond or CR₅R₆ then Z is heteroaryl or aryl; and with the proviso that the compound is not:

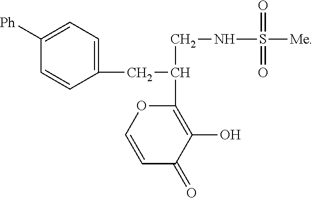 [SMILES: CS(=O)(=O)NCC(Cc1ccc(-c2ccccc2)cc1)c1occc(=O)c1O]

2. The compound of claim 1, wherein L₁ is —O—.

3. The compound of claim 1, wherein L₁ is a bond.

4. The compound of any one of claims 1-3, wherein R₁ is an alkyl, haloalkyl, cycloalkyl, alkylene-alkoxy, —NR₇R₈.

5. The compound of claim 4, wherein R₁ is an alkyl, haloalkyl, or —NR₇R₈.

6. The compound of claim 4, wherein R₁ is methyl.

7. The compound of any one of claims 1-5, wherein R₁ is —NR₇R₈.

8. The compound of claim 7, wherein R₇ and R₈ are alkyl.

9. The compound of any one of claims 1-8, wherein R₂ and R₃ are independently hydrogen or alkyl and R₄ is hydrogen.

10. The compound of any one of claims 1-9, wherein R₂, R₃, R₄ are hydrogen.

11. The compound of any one of claims 1-10, wherein Y is 3-7 membered monocyclic cycloalkyl, 5-8 membered bicyclic cycloalkyl, 4-7 membered saturated heterocyclyl, 5-8 membered bicyclic heterocyclyl, 5-6-membered heteroaryl or phenyl.

12. The compound of any one of claims 1-11, wherein Z is 5-10 membered heteroaryl or phenyl.

13. The compound of claim 12, wherein Z is phenyl substituted with one or two halogens.

14. The compound of any one of claims 1-12, wherein Y and Z are phenyl.

15. The compound of any one of claims 1-12, wherein Y is cyclohexyl and Z is phenyl.

16. The compound of claim 11, wherein Y is a 2-oxaspiro[4.5]decane or cyclohexyl optionally substituted one or more alkyl or cycloalkyl.

17. The compound of claim 14, wherein Y and Z together are:

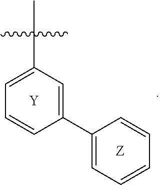

18. The compound of any one of claims 1-17, wherein A is a substituted or unsubstituted monocyclic or bicyclic nitrogen-containing heteroaryl.

19. The compound of claim 18, wherein A is selected from the group consisting of: 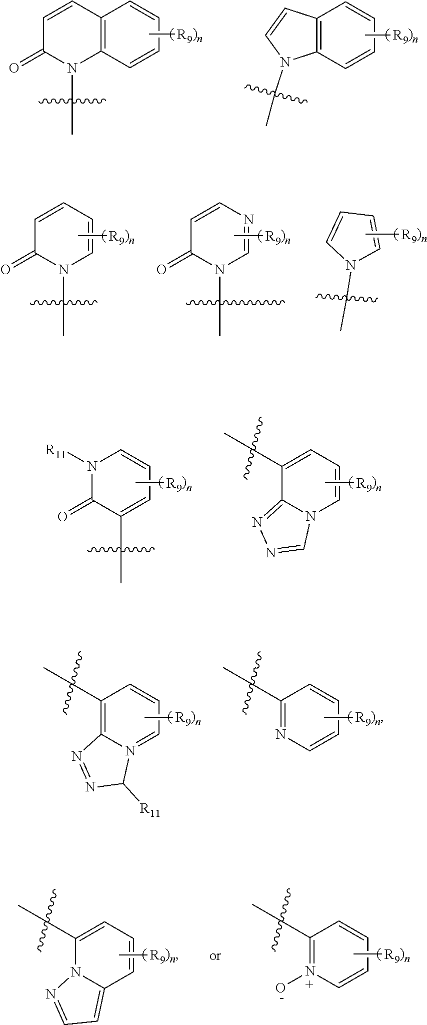
wherein n is an integer from 0-6;
R₉ is hydrogen, alkyl, haloalkyl, haloalkoxy, halogen, cyano, alkoxy, cycloalkyl, heterocyclyl, or heteroaryl; and
R₁₁ is alkyl optionally substituted with cyano, haloalkyl, cycloalkyl or heterocyclyl.

20. The compound of claim 19, wherein n is 1.

21. The compound of claim 19, wherein n is 2.

22. The compound of claim 19, wherein A is

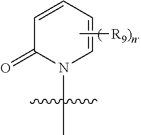

23. The compound of claim 19, wherein A is

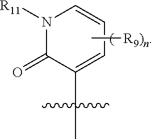

24. The compound of any one of claims 19-23, wherein R₉ is alkyl or halogen.

25. The compound of any one of claims 19-24, wherein R₁₁ is alkyl or haloalkyl.

26. The compound of claim 1, wherein the compound of Formula (I) is selected from the group consisting of compounds in Table 1.

27. A pharmaceutical composition comprising a compound of any one of claims 1-26 and pharmaceutically acceptable excipient.

28. A method of treating a disease or disorder that is treatable by administration of an Orexin agonist, the method comprising administering a therapeutically effective amount of the compound of any one of claims 1-26 or the composition of claim 27.

29. A method of treating a sleep disorder in a subject in need thereof, comprising administering to the subject a therapeutically effective amount of the compound of any one of claims 1-26 or the composition of claim 27.

30. A method for treating narcolepsy in a subject in need thereof, comprising administering to the subject an effective amount of the compound of the compound of any one of claims 1-26 or the composition of claim 27.

31. A method for treating hypersomnia in a subject in need thereof, comprising administering to the subject an effective amount of the compound of the compound of any one of claims 1-26 or the composition of claim 27.

32. A method for decreasing or treating excessive sleepiness in a subject in need thereof, comprising administering to the subject an effective amount of the compound of the compound of any one of claims 1-26 or the composition of claim 27.
